(19) United States
(12) Patent Application Publication
(10) Pub. No.: US 2024/0417476 A1
(43) Pub. Date: Dec. 19, 2024

(54) COMPOSITIONS AND METHODS COMPRISING ANTI-NRP2a ANTIBODIES

(71) Applicant: aTyr Pharma, Inc., San Diego, CA (US)

(72) Inventors: Luke G. BURMAN, San Diego, CA (US); Yeeting CHONG, San Diego, CA (US); Kaitlyn RAUCH, San Diego, CA (US); Leslie A. NANGLE, San Diego, CA (US)

(21) Appl. No.: 18/703,912

(22) PCT Filed: Oct. 27, 2022
(86) PCT No.: PCT/US2022/078780

§ 371 Date: Apr. 23, 2024

Related U.S. Application Data
(60) Provisional application No. 63/272,374, filed on Oct. 27, 2021.

(51) Int. Cl. C07K 16/28

(52) U.S. Cl. CPC C07K 16/2863; C07K 2317/24; C07K 2317/33; C07K 2317/34; C07K 2317/565; C07K 2317/76; C07K 2317/92

(57) ABSTRACT

Provided are antibodies and antigen-binding fragments thereof that preferentially or selectively bind to human neuropilin-2a (NRP2a) variant 1 (v1) and/or variant 2 (v2), relative to other NRP2a isoforms or NRP2b isoforms, and which modulate binding interactions between NRP2a v1/v2 ligands and downstream signaling events. Also included are related therapeutic compositions and methods for treating diseases such as cancers and inflammatory and autoimmune diseases.

### CROSS-REFERENCE TO RELATED APPLICATIONS

This application claims the benefit under 35 U.S.C. § 119(e) of U.S. Provisional Application No. 63/272,374, filed Oct. 27, 2021, which is incorporated by reference in its entirety.

### STATEMENT REGARDING THE SEQUENCE LISTING

The Sequence Listing XML associated with this application is provided in XML file format and is hereby incorporated by reference into the specification. The name of the XML file containing the Sequence Listing XML is ATYR_137_01WO_ST26.xml. The XML file is about 222,086 bytes, was created on Oct. 26, 2022, and is being submitted electronically via USPTO Patent Center.

### BACKGROUND

### Technical Field

The present disclosure relates to antibodies and antigen-binding fragments thereof that preferentially or selectively bind to human neuropilin-2a (NRP2a) variant 1 (v1) and/or variant 2 (v2) isoforms, relative to NRP2b isoforms, and which selectively modulate binding interactions between human NRP2a v1/v2 ligands and downstream signaling events. Also included are related therapeutic compositions and methods for treating diseases such as cancers and inflammatory and autoimmune diseases.

### Description of the Related Art

NRP2 is a single pass transmembrane protein that forms heterodimeric complexes with a large number of other plasma membrane receptors including growth factor receptors like FLT-4, KDR, and cMET (Nararre et al., (2014) OncoTargets and Therapy doi:10.2147/OTT.S377744), as well as integrins and other signaling systems (Goel et al., J. Cell Sci 125 597-506, 2012). The extracellular domain of NRP2 binds ligands of the VEGF and semaphorin families, while its short intracellular domain participates in a variety of protein-protein interactions. These interactions appear to enhance or modulate the intracellular trafficking and signaling output of growth factor receptors, integrins, and other co-receptors to facilitate cellular plasticity (Favier et al., Blood doi:10.1182/blood-2005-11-4447), among other processes that enable the cell to rapidly respond and adapt to changing environments and cellular stress.

NRP2 is typically expressed in vivo as a mixture of various closely related splice variants, which are typically grouped together as NRP2a, which comprises variants v1, v2 and v3, and NRP2b, which comprises variants v4 and v5. Variant v6 is a soluble form of NRP2 which is found within the circulation. While NRP2a and NRP2b share sequence identity over most (but not all) of their surface exposed domains, the NRP2a and NRP2b variants differ significantly in their juxtamembrane, transmembrane, and intracellular C terminal regions. In all three splice variants of NRP2a, the unique c-terminal domain comprises 42 amino acids and an intracellular PDZ-binding domain with the C-terminal SEA amino acid sequence motif. By contrast, the two splice variants of NRP2b contain a 46 amino acid cytoplasmic domain region lacking the C-terminal SEA. NRP2a and NRP2b share only about 11% sequence homology between their intracellular, juxtamembrane, and transmembrane sequences.

Increasing evidence suggests that the NRP2a and NRP2b splice variants of NRP2 play distinct roles in directing differential patterns of intracellular localization and signal transduction, to regulate cellular function. Emerging data suggests that the ratio of the expression of NRP2a and NRP2b varies over time in a specific tissue based on cellular stress and activation state, in both normal and pathophysiology states. Accordingly, the development of NRP2a and NRP2b selective antibodies offers the opportunity to develop a new generation of more selective and potent therapeutic and diagnostic agents.

However, a major limitation to developing isoform specific therapeutic and diagnostic reagents however has been the lack of suitable isoform selective, high affinity, and function blocking antibodies. This limitation has been overcome by the development and validation of NRP2a-specific antibodies described herein.

### BRIEF SUMMARY

Embodiments of the present disclosure include an antibody, or an antigen-binding fragment thereof, which binds to a neuropilin-2A (NRP2a) variant 1 (v1) or variant 2 (v2) polypeptide at an epitope that comprises, consists, or consists essentially of a sequence selected from Table N2, including about or at least about 8, 9, 10, 11, or 12 or more contiguous amino acids of a sequence selected from Table N2. In some embodiments, the epitope comprises, consists, or consists essentially of a sequence selected from SEQ ID NO: 96-104, including about or at least about 8, 9, 10, 11, or 12 contiguous amino acids of a sequence selected from SEQ ID NOs: 96-104. In specific embodiments, the epitope comprises, consists, or consists essentially of SEQ ID NO: 100, or about or at least about 8, 9, 10, 11, or 12 contiguous amino acids of SEQ ID NO: 100.

In certain embodiments, an antibody, or antigen-binding fragment thereof, comprises a heavy chain variable region (VH) sequence that comprises complementary determining region VHCDR1, VHCDR2, and VHCDR3 sequences, and a light chain variable region (VL) sequence that comprises complementary determining region VLCDR1, VLCDR2, and VLCDR3 sequences, wherein:
the VHCDR1, VHCDR2, and VHCDR3 sequences comprise SEQ ID NOs: 13, 127, and GX₁X₂X₃X₄X, (wherein X₁ is G, A, or S, X₂ is Y, F, K, L, or R, X₃ is T, A, G, I, L, Q, or V, X, is D, A, G, K, N, Q, R, or S, and X₅ is Y, A, D, E, F, G, H, I, K, L, N, Q, R, S, T, or V), respectively, and the VLCDR1, VLCDR2, and VLCDR3 sequences comprise SEQ ID NOs: 16, 17, and X₆X₇X₈X₉X₁₀X₁₁X₁₂X₁₃ (wherein X₆ is S, A, G, I, L, P, T, or V, X₇ is Q, A, G, R, or S, X₈ is S, A, H, K, L, Q, or T, X₉ is T, F, G, H, I, K, L, N, Q, R, S, V, or Y, X₁₀ is H, A, D, E, F, G, I, K, L, N, Q, R, S, T, or Y, X₁₁ is V, A, E, F, G, H, I, K, L, N, P, Q, R, S, T, or Y, X₁₂ is L, A, E, H, I, N, P, Q, S, T, or V, and X₁₃ is T, A, D, E, F, G, I, K, L, N, Q, R, S, or V), respectively;
the VHCDR1, VHCDR2, and VHCDR3 sequences comprise SEQ ID NOs: 130-132, respectively, and the VLCDR1, VLCDR2, and VLCDR3 sequences comprise SEQ ID NOs: 133-135, respectively, including variants thereof having 1, 2, 3, 4, 5, or 6 total alterations across all of the CDR regions;
the VHCDR1, VHCDR2, and VHCDR3 sequences comprise SEQ ID NOs: 136-138, respectively, and the VLCDR1, VLCDR2, and VLCDR3 sequences comprise SEQ ID NOs: 139-141, respectively, including variants thereof having 1, 2, 3, 4, 5, or 6 total alterations across all of the CDR regions;
the VHCDR1, VHCDR2, and VHCDR3 sequences comprise SEQ ID NOs: 142-144, respectively, and the VLCDR1, VLCDR2, and VLCDR3 sequences comprise SEQ ID NOs: 145-147, respectively, including variants thereof having 1, 2, 3, 4, 5, or 6 total alterations across all of the CDR regions;
the VHCDR1, VHCDR2, and VHCDR3 sequences comprise SEQ ID NOs: 148-150, respectively, and the VLCDR1, VLCDR2, and VLCDR3 sequences comprise SEQ ID NOs: 151-153, respectively, including variants thereof having 1, 2, 3, 4, 5, or 6 total alterations across all of the CDR regions;
the VHCDR1, VHCDR2, and VHCDR3 sequences comprise SEQ ID NOs: 154-156, respectively, and the VLCDR1, VLCDR2, and VLCDR3 sequences comprise SEQ ID NOs: 157-159, respectively, including variants thereof having 1, 2, 3, 4, 5, or 6 total alterations across all of the CDR regions;
the VHCDR1, VHCDR2, and VHCDR3 sequences comprise SEQ ID NOs: 1-3, respectively, and the VLCDR1, VLCDR2, and VLCDR3 sequences comprise SEQ ID NOs: 4-6, respectively, including variants thereof having 1, 2, 3, 4, 5, or 6 total alterations across all of the CDR regions; the VHCDR1, VHCDR2, and VHCDR3 sequences comprise SEQ ID NOs: 7-9, respectively, and the VLCDR1, VLCDR2, and VLCDR3 sequences comprise SEQ ID NOs: 10-12, respectively, including variants thereof having 1, 2, 3, 4, 5, or 6 total alterations across all of the CDR regions; the VHCDR1, VHCDR2, and VHCDR3 sequences comprise SEQ ID NOs: 13-15 respectively, and the VLCDR1, VLCDR2, and VLCDR3 sequences comprise SEQ ID NOs: 16-18, respectively, including variants thereof having 1, 2, 3, 4, 5, or 6 total alterations across all of the CDR regions;
the VHCDR1, VHCDR2, and VHCDR3 sequences comprise SEQ ID NOs: 19-21, respectively, and the VLCDR1, VLCDR2, and VLCDR3 sequences comprise SEQ ID NOs: 22-24, respectively, including variants thereof having 1, 2, 3, 4, 5, or 6 total alterations across all of the CDR regions;
the VHCDR1, VHCDR2, and VHCDR3 sequences comprise SEQ ID NOs: 25-27, respectively, and the VLCDR1, VLCDR2, and VLCDR3 sequences comprise SEQ ID NOs: 28-30, respectively, including variants thereof having 1, 2, 3, 4, 5, or 6 total alterations across all of the CDR regions;
the VHCDR1, VHCDR2, and VHCDR3 sequences comprise SEQ ID NOs: 31-33, respectively, and the VLCDR1, VLCDR2, and VLCDR3 sequences comprise SEQ ID NOs: 34-36, respectively, including variants thereof having 1, 2, 3, 4, 5, or 6 total alterations across all of the CDR regions;
the VHCDR1, VHCDR2, and VHCDR3 sequences comprise SEQ ID NOs: 37-39, respectively, and the VLCDR1, VLCDR2, and VLCDR3 sequences comprise SEQ ID NOs: 40-42, respectively, including variants thereof having 1, 2, 3, 4, 5, or 6 total alterations across all of the CDR regions;
the VHCDR1, VHCDR2, and VHCDR3 sequences comprise SEQ ID NOs: 43-45, respectively, and the VLCDR1, VLCDR2, and VLCDR3 sequences comprise SEQ ID NOs: 46-48, respectively, including variants thereof having 1, 2, 3, 4, 5, or 6 total alterations across all of the CDR regions;
the VHCDR1, VHCDR2, and VHCDR3 sequences comprise SEQ ID NOs: 49-51, respectively, and the VLCDR1, VLCDR2, and VLCDR3 sequences comprise SEQ ID NOs: 52-54, respectively, including variants thereof having 1, 2, 3, 4, 5, or 6 total alterations across all of the CDR regions;
the VHCDR1, VHCDR2, and VHCDR3 sequences comprise SEQ ID NOs: 55-57, respectively, and the VLCDR1, VLCDR2, and VLCDR3 sequences comprise SEQ ID NOs: 58-60, respectively, including variants thereof having 1, 2, 3, 4, 5, or 6 total alterations across all of the CDR regions; or the VHCDR1, VHCDR2, and VHCDR3 sequences comprise SEQ ID NOs: 61-63, respectively, and the VLCDR1, VLCDR2, and VLCDR3 sequences comprise SEQ ID NOs: 64-66, respectively, including variants thereof having 1, 2, 3, 4, 5, or 6 total alterations across all of the CDR regions.

### In Particular Embodiments:

the V_{H} sequence comprises SEQ ID NO: 170, and the V_{L} sequence comprises SEQ ID NO: 171;
the V_{H} sequence is at least 80, 85, 90, 95, 97, 98, 99, or 100% identical to SEQ ID NO: 160, and the V_{L} sequence is at least 80, 85, 90, 95, 97, 98, 99, or 100% identical to SEQ ID NO: 161;
the V_{H} sequence is at least 80, 85, 90, 95, 97, 98, 99, or 100% identical to SEQ ID NO: 162, and the V_{L} sequence is at least 80, 85, 90, 95, 97, 98, 99, or 100% identical to SEQ ID NO: 163;
the V_{H} sequence is at least 80, 85, 90, 95, 97, 98, 99, or 100% identical to SEQ ID NO: 164, and the V_{L} sequence is at least 80, 85, 90, 95, 97, 98, 99, or 100% identical to SEQ ID NO: 165;
the V_{H} sequence is at least 80, 85, 90, 95, 97, 98, 99, or 100% identical to SEQ ID NO: 166, and the V_{L} sequence is at least 80, 85, 90, 95, 97, 98, 99, or 100% identical to SEQ ID NO: 167;
the V_{H} sequence is at least 80, 85, 90, 95, 97, 98, 99, or 100%/6 identical to SEQ ID NO: 168, and the V_{L} sequence is at least 80, 85, 90, 95, 97, 98, 99, or 100% identical to SEQ ID NO: 169;
the V_{H} sequence is at least 80, 85, 90, 95, 97, 98, 99, or 100% identical to SEQ ID NO: 67, and the V_{L} sequence is at least 80, 85, 90, 95, 97, 98, 99, or 100% identical to SEQ ID NO: 68;
the V_{H} sequence is at least 80, 85, 90, 95, 97, 98, 99, or 100% identical to SEQ ID NO: 69, and the V_{L} sequence is at least 80, 85, 90, 95, 97, 98, 99, or 100% identical to SEQ ID NO: 70;
the V_{H} sequence is at least 80, 85, 90, 95, 97, 98, 99, or 100% identical to SEQ ID NO: 71, and the V_{L} sequence is at least 80, 85, 90, 95, 97, 98, 99, or 100% identical to SEQ ID NO: 72;
the V_{H} sequence is at least 80, 85, 90, 95, 97, 98, 99, or 100% identical to SEQ ID NO: 73, and the V_{L} sequence is at least 80, 85, 90, 95, 97, 98, 99, or 100% identical to SEQ ID NO: 74;
the V_{H} sequence is at least 80, 85, 90, 95, 97, 98, 99, or 100% identical to SEQ ID NO: 75, and the V_{L} sequence is at least 80, 85, 90, 95, 97, 98, 99, or 100% identical to SEQ ID NO: 76;
the V_{H} sequence is at least 80, 85, 90, 95, 97, 98, 99, or 100% identical to SEQ ID NO: 77, and the V_{L} sequence is at least 80, 85, 90, 95, 97, 98, 99, or 100% identical to SEQ ID NO: 78;
the V_{H} sequence is at least 80, 85, 90, 95, 97, 98, 99, or 100% identical to SEQ ID NO: 79, and the V_{L} sequence is at least 80, 85.90, 95, 97, 98, 99, or 100% identical to SEQ ID NO: 80;
the V_{H} sequence is at least 80, 85, 90, 95, 97, 98, 99, or 100% identical to SEQ ID NO: 81, and the V_{L} sequence is at least 80, 85, 90, 95, 97, 98, 99, or 100% identical to SEQ ID NO: 82;
the V_{H} sequence is at least 80, 85, 90, 95, 97, 98, 99, or 100% identical to SEQ ID NO: 83, and the V_{L} sequence is at least 80, 85, 90, 95, 97, 98, 99, or 100% identical to SEQ ID NO: 84;
the V_{H} sequence is at least 80, 85, 90, 95, 97, 98, 99, or 100% identical to SEQ ID NO: 85, and the V_{L} sequence is at least 80, 85, 90, 95, 97, 98, 99, or 100% identical to SEQ ID NO: 86; or
the V_{H} sequence is at least 80, 85, 90, 95, 97, 98, 99, or 100% identical to SEQ ID NO: 87, and the V_{L} sequence is at least 80, 85, 90, 95, 97, 98, 99, or 100% identical to SEQ ID NO: 88.

In some embodiments, an antibody, or antigen-binding fragment thereof, does not substantially bind to a human neuropilin-2B (NRP2b) variant 4 (v4) polypeptide, and/or a human NRP2b variant 5 (v5) polypeptide. In some embodiments, an antibody, or antigen-binding fragment thereof, binds to the NRP2a v1 or v2 polypeptide, or the epitope, with an affinity of about 10 pM to about 500 pM or to about 50 nM, or about, at least about, or no more than about 10, 20, 30, 40, 50, 60, 70, 80, 90, 100, 110, 120, 130, 140, 150, 160, 170, 180, 190, 200, 300, 400, 500, 600, 700, 800, 900 pM, 1 nM, 10 nM, 25 nM, or 50 nM, or optionally with an affinity that ranges from about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500 pM, about 30 pM to about 400 pM, about 30 pM to about 300 pM, about 30 pM to about 200 pM, about 30 pM to about 100 pM, about 30 pM to about 50 pM, or about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, about 100 pM to about 1 nM, about 1 nM to about 5 nM, about 5 nM to about 10 nM, about 10 nM to 25 nM, or about 25 nM to about 50 nM.

In certain embodiments, binding affinity of the antibody, or antigen-binding fragment thereof, for the NRP2a v1 or v2 polypeptide is at least about 1.5, 2, 4, 6, 8, 10, 20, 40, 60, 80, 100, 200, 400, 600, 800, or 1000 times stronger that its binding affinity for a NRP2b v4 poly peptide, and/or a NRP2b v5 polypeptide.

In some embodiments, an antibody, or antigen-binding fragment thereof, blocks or otherwise reduces binding between the NRP2a v1 or v2 polypeptide and a ligand thereof, optionally wherein the ligand is selected from Table L1 or Table L2. In some embodiments, an antibody, or antigen-binding fragment thereof, blocks or otherwise reduces binding between the NRP2a v1 or v2 polypeptide a chemokine (C—C motif) ligand 21 (CCL21) polypeptide, optionally in an in vitro binding assay, an in vitro or ex vivo cell-based assay, or in vivo. In some embodiments, an antibody, or antigen-binding fragment thereof, blocks or otherwise reduces binding between the NRP2a v1 or v2 polypeptide and the CCL21 polypeptide by about or at least about 20-100% or more (optionally about 20, 25, 30, 35, 40, 45, 50, 55, 60, 65, 70, 75, 80, 90, or 100% or more) relative to a control or reference. In specific embodiments, an antibody, or antigen-binding fragment thereof, blocks or otherwise reduces binding, including dimerization, between the NRP2a v1 or v2 polypeptide and a C—C chemokine receptor type 7 (CCR7) polypeptide, optionally in an in vitro binding assay, an in vitro or ex vivo cell-based assay, or in vivo. In particular embodiments, an antibody, or antigen-binding fragment thereof, blocks or otherwise reduces binding, including dimerization, between the NRP2a v1 or v2 poly peptide and the CCR7 polypeptide by about or at least about 20-100% or more (optionally about 20, 25, 30, 35, 40, 45, 50, 55, 60, 65, 70, 75, 80, 90, or 100% or more) relative to a control or reference.

In some embodiments, an antibody, or antigen-binding fragment thereof, modulates (optionally antagonizes) the signaling activity between the NRP2a v1 or v2 polypeptide and a CCL21 and/or CCR7 poly peptide, optionally by about or at least about 20-100% or more (optionally about 20, 25, 30, 35, 40, 45, 50, 55, 60, 65, 70, 75, 80, 90, or 100% or more) relative to a control or reference. In certain embodiments, the signaling activity comprises induction of immune cell migration, optionally dendritic cells or mature T-cells, and wherein the antibody, or antigen-binding fragment thereof, reduces the signaling activity; and/or wherein the signaling activity comprises induction of tumor cell migration, and wherein the antibody, or antigen-binding fragment thereof, reduces the signaling activity.

In some embodiments, an antibody, or antigen-binding fragment thereof, comprises an IgA (including subclasses IgA1 and IgA2), IgD, IgE, IgG (including subclasses IgG1, IgG2, IgG3, and IgG4), or IgM Fc domain, optionally a human Fc domain, or a hybrid and/or variant thereof. In some embodiments, an antibody, or antigen-binding fragment thereof, comprises an IgG Fc domain with high effector function in humans, optionally an IgG1 or IgG3 Fc domain; or which comprises an IgG Fc domain with low effector function in humans, optionally an IgG2 or IgG4 Fc domain. In some embodiments, an antibody, or antigen-binding fragment thereof, comprises an IgG1 or IgG4 Fc domain, optionally selected from Table F1.

Certain embodiments include a monoclonal antibody, a humanized antibody, an Fv fragment, a single chain Fv (scFv) polypeptide, an adnectin, an anticalin, an aptamer, an avimer, a camelid antibody, a designed ankyrin repeat protein (DARPin), a minibody, a nanobody, and/or a unibody.

Also included are therapeutic compositions, comprising a pharmaceutically-acceptable carrier and an antibody, or antigen-binding fragment thereof, as described herein. In some embodiments, the composition has a purity of at least about 80%, 85%, 90%, 95%, 98%, or 99% on a protein basis with respect to the at least one antibody, or antigen-binding fragment thereof, and is substantially aggregate-free. In some embodiments, the therapeutic composition is substantially endotoxin-free. In some embodiments, the therapeutic composition is a sterile, injectable solution, optionally suitable for intravenous, intramuscular, subcutaneous, or intraperitoneal administration. Certain therapeutic compositions and methods further comprising at least one additional agent selected from one or more of a cancer immunotherapy agent, a chemotherapeutic agent, a hormonal therapeutic agent, and a kinase inhibitor.

Also included are methods of treating a disease or condition in a subject in need thereof, comprising administering to the subject a therapeutic composition described herein. In some embodiments, the disease or condition is a neuropilin 2 (NRP2)-associated disease or condition, optionally an NRP2a-associated disease or condition. In some embodiments, the disease or condition is selected from a cancer, an inflammatory disease, an autoimmune disease, a lymphatic disease or associated condition, a fibrotic disease, and a disease associated with reduced smooth muscle contractility.

In certain embodiments, the disease is a cancer, optionally wherein the cancer expresses or overexpresses NRP2, optionally wherein the cancer displays NRP2-dependent growth, NRP2-dependent adhesion, NRP2-dependent migration, and/or NRP2-dependent invasion. In specific embodiments, the cancer expresses or overexpresses NRP2 but does not substantially express neuropilin-1 (NRP1). Certain methods are for reducing or preventing re-emergence of a cancer in a subject in need thereof, wherein administration of the therapeutic composition enables generation of an immune memory to the cancer. In some embodiments, the subject has lymphedema.

Certain embodiments include administering to the subject at least one additional agent selected from one or more of a cancer immunotherapy agent, a chemotherapeutic agent, a hormonal therapeutic agent, and a kinase inhibitor. In some embodiments, the at least one anti-NRPa2 antibody or antigen-binding fragment thereof and the at least one agent are administered separately, as separate compositions. In some embodiments, the at least one anti-NRP2 antibody and the at least one agent are administered together as part of the same therapeutic composition, optionally as a therapeutic composition described herein.

In some embodiments, the cancer immunotherapy agent is selected from one or more of an immune checkpoint modulatory agent, a cancer vaccine, an oncolytic virus, a cytokine, and a cell-based immunotherapies. In certain embodiments, the immune checkpoint modulatory agent is a polypeptide, optionally an antibody or antigen-binding fragment thereof or a ligand, or a small molecule. In some embodiments, the immune checkpoint modulatory agent comprises
(a) an antagonist of a inhibitory immune checkpoint molecule; or
(b) an agonist of a stimulatory immune checkpoint molecule.
optionally, wherein the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT). In particular embodiments:
the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736), optionally wherein the cancer is selected from one or more of colorectal cancer, melanoma, breast cancer, non-small-cell lung carcinoma, bladder cancer, and renal cell carcinoma;
the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab, optionally wherein the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer;
the PD-1 antagonist is pembrolizumab and the cancer is optionally selected from one or more of melanoma, non-small cell lung cancer, small cell lung cancer, head and neck cancer, and urothelial cancer;
the antagonist is a CTLA-4 antagonist optionally selected from one or more of an antibody or antigen-binding fragment or small molecule that specifically binds thereto, ipilimumab, tremelimumab, optionally wherein the cancer is selected from one or more of melanoma, prostate cancer, lung cancer, and bladder cancer;
the antagonist is an IDO antagonist optionally selected from one or more of an antibody or antigen-binding fragment or small molecule that specifically binds thereto, indoximod (NLG-8189), 1-methyl-tryptophan (1 MT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor;
the antagonist is a TDO antagonist optionally selected from one or more of an antibody or antigen-binding fragment or small molecule that specifically binds thereto, 680C91, and LM10;
the antagonist is a TIM-3 antagonist optionally selected from one or more of an antibody or antigen-binding fragment or small molecule that specifically binds thereto;
the antagonist is a LAG-3 antagonist optionally selected from one or more of an antibody or antigen-binding fragment or small molecule that specifically binds thereto, and BMS-986016;
the antagonist is a VISTA antagonist optionally selected from one or more of an antibody or antigen-binding fragment or small molecule that specifically binds thereto;
the antagonist is a BTLA, CD160, and/or HVEM antagonist optionally selected from one or more of an antibody or antigen-binding fragment or small molecule that specifically binds thereto;
the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM). In certain embodiments:
the agonist is an OX40 agonist optionally selected from one or more of an antibody or antigen-binding fragment or small molecule or ligand that specifically binds thereto, OX86, Fc-OX40L, and GSK3174998;
the agonist is a CD40 agonist optionally selected from one or more of an antibody or antigen-binding fragment or small molecule or ligand that specifically binds thereto, CP-870,893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma;
the agonist is a GITR agonist optionally selected from one or more of an antibody or antigen-binding fragment or small molecule or ligand that specifically binds thereto, INCAGN01876, DTA-1, and MEDI1873;
the agonist is a CD137 agonist optionally selected from one or more of an antibody or antigen-binding fragment or small molecule or ligand that specifically binds thereto, utomilumab, and 4-1BB ligand;
the agonist is a CD27 agonist optionally selected from one or more of an antibody or antigen-binding fragment or small molecule or ligand that specifically binds thereto, varlilumab, and CDX-1127 (1F5);
the agonist is a CD28 agonist optionally selected from one or more of an antibody or antigen-binding fragment or small molecule or ligand that specifically binds thereto, and TAB08; and/or
the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2). SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI). Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401. In some embodiments, the cytokine selected from one or more of interferon (IFN)-α, IL-2, IL-12, IL-7, IL-21, and Granulocyte-macrophage colony-stimulating factor (GM-CSF). In some embodiments, the cell-based immunotherapy agent comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

In particular embodiments, the at least one chemotherapeutic agent is selected from one or more of an alkylating agent, an anti-metabolite, a cytotoxic antibiotic, a topoisomerase inhibitor (type 1 or type 11), and an anti-microtubule agent. In some embodiments:
the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);
the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);
the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;
the topoisomerase inhibitor is selected from one or more of camptothecin, irinotecan, topotecan, etoposide, doxorubicin, mitoxantrone, teniposide, novobiocin, merbarone, and aclarubicin; and/or
the anti-microtubule agent is selected from one or more of taxanes (optionally paclitaxel and docetaxel) and vinca alkaloids (optionally vinblastine, vincristine, vindesine, vinorelbine).

In some embodiments, the at least one hormonal therapeutic agent is a hormonal agonist or a hormonal antagonist. In certain embodiments, the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog. In some embodiments, the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afatinib, aflibercept, axitinib, bevacizumab, bosutinib, cabozantinib, cetuximab, cobimetinib, crizotinib, dasatinib, entrectinib, erdafitinib, erlotinib, fostamatinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemurafenib.

In some embodiments, the cancer is a primary cancer. In certain embodiments, the cancer is a metastatic cancer, optionally a metastatic cancer that expresses NRP2a and/or NRP2b.

In some embodiments, the cancer is selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma. B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, and stomach cancer.

In some embodiments, the metastatic cancer is selected from one or more of:
(a) a bladder cancer which has metastasized to the bone, liver, and/or lungs;
(b) a breast cancer which has metastasized to the bone, brain, liver, and/or lungs;
(c) a colorectal cancer which has metastasized to the liver, lungs, and/or peritoneum;
(d) a kidney cancer which has metastasized to the adrenal glands, bone, brain, liver, and/or lungs;
(e) a lung cancer which has metastasized to the adrenal glands, bone, brain, liver, and/or other lung sites;
(f) a melanoma which has metastasized to the bone, brain, liver, lung, and/or skin/muscle;
(g) a ovarian cancer which has metastasized to the liver, lung, and/or peritoneum;
(h) a pancreatic cancer which has metastasized to the liver, lung, and/or peritoneum;
(i) a prostate cancer which has metastasized to the adrenal glands, bone, liver, and/or lungs;
(j) a stomach cancer which has metastasized to the liver, lung, and/or peritoneum;
(l) a thyroid cancer which has metastasized to the bone, liver, and/or lungs; and
(m) a uterine cancer which has metastasized to the bone, liver, lung, peritoneum, and/or vagina.
   Also included are patient care kits, comprising:
(a) an antibody, or an antigen-binding fragment thereof, as described herein; and optionally
(b) at least one additional agent selected from a cancer immunotherapy agent, a chemotherapeutic agent, a hormonal therapeutic agent, and a kinase inhibitor.

In some embodiments, (a) and (b) are in separate therapeutic compositions. In some embodiments, (a) and (b) are in the same therapeutic composition. In some embodiments, the at least one chemotherapeutic agent is selected from one or more of an alkylating agent, an anti-metabolite, a cytotoxic antibiotic, a topoisomerase inhibitor (type 1 or type II), and an anti-microtubule agent.

Certain embodiments include a bioassay system, comprising an antibody, or an antigen-binding fragment thereof, as described herein, and a host cell line that expresses a human NRP2 polypeptide on the cell surface. In some embodiments, the NRP2 polypeptide is labeled with a detectable label. In some embodiments, the antibody, or antigen-binding fragment thereof, is labeled with a detectable label. In some embodiments, the NRP2 polypeptide is functionally coupled to a readout or indicator, such as a fluorescent or luminescent indicator of biological activity of the NRP2 polypeptide. In certain embodiments, the NRP2 polypeptide is selected from Table N1, optionally an NRP2a v1 and/or v2 polypeptide. Particular bioassay systems comprise at least one NRP2a ligand, optionally an NRP2a ligand selected from Table L1 or Table L2, optionally wherein the host cell expresses the at least one NRP2a ligand.

Particular embodiments include a detection system, comprising a cell that expresses a human neuropilin 2a (NRP2a) polypeptide, at least one NRP2a ligand, and a human or humanized anti-NRP2a antibody, or an antigen-binding fragment thereof, as described herein, which modulates the interaction between the NRP2a polypeptide and the at least one NRP2a ligand. In some embodiments, the anti-NRP2a antibody, or antigen-binding fragment thereof, is labeled with a detectable label. In some embodiments, the NRP2a polypeptide is a NRP2a variant 1 and/or variant 2 polypeptide selected from Table N1. In some embodiments, the at least one NRP2a ligand is selected from Table L1 or Table L2. In some embodiments, the NRP2a polypeptide and/or the at least one NRP2a ligand is/are functionally coupled to a readout or indicator, such as a fluorescent or luminescent indicator of biological activity of the NRP2a polypeptide or the at least one NRP2a ligand.

Also included are cellular compositions, comprising an engineered population of cells in which at least one cell comprises one or more polynucleotides encoding a human or humanized anti-NRP2a antibody, or antigen-binding fragment thereof, as described herein, wherein the cells are capable of growing in a serum-free medium.

Some embodiments include a cellular growth device, comprising a human or humanized anti-NRP2a antibody, or an antigen-binding fragment thereof, as described herein, an engineered population of cells in which at least one cell comprises one or more polynucleotides encoding said anti-NRP2a antibody, or antigen-binding fragment thereof, at least about 10 liters of a serum-free growth medium, and a sterile container.

### DESCRIPTION OF THE FIGURES

FIG. 1 shows a model of the interaction of NRP2a v1/v2 with CCL21 and CCR7. NRP2a v3 and NRP2b v4/v5 do not significantly interact with CCL21 or CCR7, in part because these NRP2 isoforms lack a CCL21-interacting sequence within the juxtamembrane domain.

FIGS. 2A-2E show the association between NRP2a v1/v2 isoforms with CCR7 in the presence of CCL21 or CCL19.

FIGS. 3A-3E show the effects of mutations in the CCL21-binding site of NRP2a with respect to ligand-induced dimerization of NRP2a and CCR7.

FIGS. 4A-4C show the relative binding of antibodies to human and mouse NRP2A (4A), human NRP2a and NRP2b (4B), and human NRP2a v2 and NRP2a v3 (4C).

FIGS. 5A-5D show the ability of antibodies to block NRP2a/CCR7-CCL21 induced receptor dimerization.

FIG. 6 shows sequence analysis of exemplary anti-NRP2a antibody epitopes (SEQ ID NO: 95 (NRP2a), and corresponding homologous mouse sequence SEQ ID NO: 124 (mNRP2a)).

FIGS. 7A-7B show the workflow (7A) and gating strategy (78) of an in vivo dendritic cell migration assay. FIG. 7C shows the results of FITC+ dendritic cells in the lymph node of NRP2 KO relative to wild-type mice.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. For example, an antigen can be recognized by antibodies (humoral immune response) or sensitized T-lymphocytes (T helper or cell-mediated immune response), or both. Antigens can be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

The term “anergy” refers to the functional inactivation of a T-cell, or B-cell response to re-stimulation by antigen.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, the term “antibody” encompasses not only intact polyclonal or monoclonal antibodies, but also fragments thereof (such as dAb, Fab, Fab′, F(ab′)2, Fv), single chain (scFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. Certain features and characteristics of antibodies (and antigen-binding fragments thereof) are described in greater detail herein.

An antibody or antigen-binding fragment can be of essentially any type. As is well known in the art, an antibody is an immunoglobulin molecule capable of specific binding to a target, such as an immune checkpoint molecule, through at least one epitope recognition site, located in the variable region of the immunoglobulin molecule.

The term “antigen-binding fragment” as used herein refers to a polypeptide fragment that contains at least one CDR of an immunoglobulin heavy and/or light chain that binds to the antigen of interest. In this regard, an antigen-binding fragment of the herein described antibodies may comprise 1, 2, 3, 4, 5, or all 6 CDRs of a V_{H} and V_{L} sequence from antibodies that bind to a target molecule.

The binding properties of antibodies and antigen-binding fragments thereof can be quantified using methods well known in the art (see Davies et al., Annual Rev. Biochem. 59:439-473, 1990). In some embodiments, an antibody or antigen-binding fragment thereof specifically binds to a target molecule, for example, an NRP2a v1 and/or v2 polypeptide or an epitope or complex thereof, with an equilibrium dissociation constant that is about or ranges from about ≤10⁻⁴V M to about 10⁻⁸ M. In some embodiments, the equilibrium dissociation constant is about or ranges from about ≤10⁻⁹ M to about ≤10⁻¹⁰ M. In certain illustrative embodiments, an antibody or antigen-binding fragment thereof has an affinity (Kd or EC₅₀) for a target molecule (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as a polypeptide or antibody is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope

Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd or EC₅₀. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution. In some embodiments, affinity is expressed in the terms of the half maximal effective concentration (EC₅₀), which refers to the concentration of an agent, such as an anti-NRP2a antibody, as disclosed herein, which induces a response halfway between the baseline and maximum after a specified exposure time. The EC₅₀ is commonly used as a measure of an antibody's potency.

Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a poly peptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see. e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however. FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv, fragment for use according to certain embodiments can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH:VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv (scFV) antibodies are contemplated. For example, Kappa bodies (Ill et al., Prot. Eng. 10:949-57, 1997); minibodies (Martin et al., EMBO J 13:5305-9, 1994); diabodies (Holliger et al., PNAS 90: 6444-8, 1993); or Janusins (Traunecker et al., EMBO J 10: 3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (scFv) polypeptide is a covalently linked VH:VL heterodimer which is expressed from a gene fusion including VH- and VL-encoding genes linked by a peptide-encoding linker. Huston et al. (PNAS USA. 85(16):5879-5883, 1988). A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, the antibodies or antigen-binding fragments described herein are in the form of a “diabody.” Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen-binding site: antigen-binding sites are formed by the association of the first domain of one polypeptide within the multimer with the second domain of another polypeptide within the multimer (WO94/13804). A dAb fragment of an antibody consists of a V_{H} domain (Ward et al., Nature 341:544-546, 1989). Diabodies and other multivalent or multispecific fragments can be constructed, for example, by gene fusion (see WO94/13804; and Holliger et al., PNAS USA. 90:6444-6448, 1993)).

Minibodies comprising a scFv joined to a CH3 domain are also included (see Hu et al., Cancer Res. 56:3055-3061, 1996). See also Ward et al., Nature. 341:544-546, 1989; Bird et al., Science. 242:423-426, 1988; Huston et al., PNAS USA. 85:5879-5883, 1988); PCT/US92/09965; WO94/13804; and Reiter et al., Nature Biotech. 14:1239-1245, 1996.

Where bispecific antibodies are to be used, these may be conventional bispecific antibodies, which can be manufactured in a variety of ways (Holliger and Winter, Current Opinion Biotechnol. 4:446-449, 1993), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (Ridgeway et al., Protein Eng., 9:616-621, 1996).

In certain embodiments, the antibodies or antigen-binding fragments described herein are in the form of a UniBody®. A UniBody® is an IgG4 antibody with the hinge region removed (see GenMab Utrecht, The Netherlands; see also, e.g., US20090226421). This antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies and antigen-binding fragments described herein are in the form of a nanobody. Minibodies are encoded by single genes and are efficiently produced in almost all prokaryotic and eukaryotic hosts, for example, E. coli (see U.S. Pat. No. 6,765,087), molds (for example Aspergillus or Trichoderma) and yeast (for example Saccharomyces, Kluyvermyces, Hansenula or Pichia (see U.S. Pat. No. 6,838,254). The production process is scalable and multi-kilogram quantities of nanobodies have been produced. Nanobodies may be formulated as a ready-to-use solution having a long shelf life. The Nanoclone method (see WO 06/079372) is a proprietary method for generating Nanobodies against a desired target, based on automated high-throughput selection of B-cells.

In some embodiments, the antibodies or antigen-binding fragments described herein are in the form of an aptamer (see, e.g., Ellington et al., Nature. 346, 818-22, 1990; and Tuerk et al., Science. 249, 505-10, 1990, incorporated by reference). Examples of aptamers included nucleic acid aptamers (e.g., DNA aptamers, RNA aptamers) and peptide aptamers. Nucleic acid aptamers refer generally to nucleic acid species that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. Sec. e.g., U.S. Pat. Nos. 6,376,190; and 6,387,620, incorporated by reference.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments utilize the bacterial protein Thioredoxin-A as a scaffold protein, the variable loop being inserted within the reducing active site (-Cys-Gly-Pro-Cys-loop in the wild protein), with the two cysteines lateral chains being able to form a disulfide bridge. Methods for identifying peptide aptamers are described, for example, in U.S. Application No. 2003/0108532, incorporated by reference. Peptide aptamer selection can be performed using different systems known in the art, including the yeast two-hybrid system.

In some embodiments, the antibodies or antigen-binding fragments described herein are in the form of an avimer. Avimers refer to multimeric binding proteins or peptides engineered using in vitro exon shuffling and phage display. Multiple binding domains are linked, resulting in greater affinity and specificity compared to single epitope immunoglobulin domains. See, e.g., Silverman et al., Nature Biotechnology. 23:1556-1561, 2005; U.S. Pat. No. 7,166,697; and U.S. Application Nos. 2004/0175756, 2005/0048512, 2005/0053973, 2005/0089932 and 2005/0221384, incorporated by reference.

In some embodiments, the antibodies or antigen-binding fragments described herein are in the form of an adnectin. Adnectins refer to a class of targeted biologics derived from human fibronectin, an abundant extracellular protein that naturally binds to other proteins. See, e.g., U.S. Application Nos. 2007/0082365; 2008/0139791; and 2008/0220049, incorporated by reference. Adnectins typically consists of a natural fibronectin backbone, as well as the multiple targeting domains of a specific portion of human fibronectin. The targeting domains can be engineered to enable an adnectin to specifically recognize an NRP2 poly peptide or an epitope thereof.

In some embodiments, the antibodies or antigen-binding fragments described herein are in the form of an anticalin. Anticalins refer to a class of antibody mimetics that are typically synthesized from human lipocalins, a family of binding proteins with a hypervariable loop region supported by a structurally rigid framework. See. e.g., U.S. Application No. 2006/0058510. Anticalins typically have a size of about 20 kDa. Anticalins can be characterized by a barrel structure formed by eight antiparallel β-strands (a stable β-barrel scaffold) that are pairwise connected by four peptide loops and an attached α-helix. In certain aspects, conformational deviations to achieve specific binding are made in the hypervariable loop region(s). See, e.g., Skerra. FEBS J. 275:2677-83, 2008, incorporated by reference.

In some embodiments, the antibodies or antigen-binding fragments described herein are in the form of a designed ankyrin repeat protein (DARPin). DARPins include a class of non-immunoglobulin proteins that can offer advantages over antibodies for target binding in drug discovery and drug development. Among other uses, DARPins are ideally suited for in vivo imaging or delivery of toxins or other therapeutic payloads because of their favorable molecular properties, including small size and high stability. The low-cost production in bacteria and the rapid generation of many target-specific DARPins make the DARPin approach useful for drug discovery. Additionally, DARPins can be easily generated in multispecific formats, offering the potential to target an effector DARPin to a specific organ or to target multiple receptors with one molecule composed of several DARPins. See. e.g., Stumpp et al., Curr Opin Drug Discov Devel. 10:153-159, 2007; U.S. Application No. 2009/0082274; and PCT/EP2001/10454, incorporated by reference.

Also included are heavy chain dimers, such as antibodies from camelids and sharks. Camelid and shark antibodies comprise a homodimeric pair of two chains of V-like and C-like domains (neither has a light chain). Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called VHH domains. Shark Ig-NARs comprise a homodimer of one variable domain (termed a V-NAR domain) and five C-like constant domains (C-NAR domains).

In camelids, the diversity of antibody repertoire is determined by the complementary determining regions (CDR) 1, 2, and 3 in the V_{H} or VHH regions. The CDR3 in the camel VHH region is characterized by its relatively long length averaging 16 amino acids (Muyldermans et al., 1994. Protein Engineering 7(9): 1129). This is in contrast to CDR3 regions of antibodies of many other species. For example, the CDR3 of mouse V_{H} has an average of 9 amino acids. Libraries of camelid-derived antibody variable regions, which maintain the in vivo diversity of the variable regions of a camelid, can be made by, for example, the methods disclosed in U.S. patent application Ser. No. 20050037421, published Feb. 17, 2005 In certain embodiments, the antibodies or antigen-binding fragments thereof are humanized.

These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeven et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies are “chimeric” antibodies. In this regard, a chimeric antibody is comprised of an antigen-binding fragment of an antibody operably linked or otherwise fused to a heterologous Fc portion of a different antibody. In certain embodiments, the Fc domain or heterologous Fc domain is of human origin. In certain embodiments, the Fc domain or heterologous Fc domain is of mouse origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, V_{H} or both).

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

The term “chemoresistance” refers to the change in therapeutic sensitivity of a cancer cell population over time following exposure to chemotherapy, including resistance to at least one of a cancer immunotherapy agent, a chemotherapeutic agent, a hormonal therapeutic agent, and/or a kinase inhibitor. Eventually, chemoresistance leads to the relapse and/or metastasis, of the cancer, and challenges the improvement of clinical outcome for the cancer patients. It remains the main obstacle to long term successful cancer therapy. For example, approximately 30 percent of women diagnosed with early-stage breast cancer ultimately develop resistance and eventually progress to metastatic breast cancer. The molecular mechanisms of chemoresistance include the induction of transporter pumps, oncogenes, tumor suppressor genes, mitochondrial alteration, DNA repair, autophagy, epithelial-mesenchymal transition (EMT), cancer stemness, and exosome production. These processes may operate via distinct mechanisms, alone or in combination with each other, but ultimately coordinate to prevent cell death in response to a specific targeted chemotherapeutic agent. For example, such processes provide alternative pro-growth signals and/or eliminate or otherwise reduce apoptotic pathways. Accordingly, agents that reduce chemoresistance could find utility in the treatment or reduction of chemoresistant cancers.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “effector function”, or “ADCC effector function” in the context of antibodies refers to the ability of that antibody to engage with other arms of the immune system, including for example, the activation of the classical complement pathway, or through engagement of Fc receptors. Complement dependent pathways are primarily driven by the interaction of C1q with the C1 complex with clustered antibody Fc domains. Antibody dependent cellular cytotoxicity (ADCC), is primarily driven by the interaction of Fc receptors (FcRs) on the surface of effector cells (natural killer cells, macrophages, monocytes and eosinophils) which bind to the Fc region of an IgG which itself is bound to a target cell. Fc receptors (FcRs) are key immune regulatory receptors connecting the antibody mediated (humoral) immune response to cellular effector functions. Receptors for all classes of immunoglobulins have been identified, including FcγR (IgG), FcεR1 (IgE), FcαR1 (IgA), FcμR (IgM) and FcδR (IgD). There are at least three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range KD) while FcγRII and FcγRIII are low to intermediate affinity (micromolar range KD). Upon Fc binding a signaling pathway is triggered which results in the secretion of various substances, such as lytic enzymes, perform, granzymes and tumour necrosis factor, which mediate in the destruction of the target cell. The level of ADCC effector function various for human IgG subtypes. Although this is dependent on the allotype and specific FcvR, in simple terms ADCC effector function is “high” for human IgG1 and IgG3, and “low” for IgG2 and IgG4.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C., and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “epitope” includes any determinant, preferably a polypeptide determinant, capable of specific binding to an immunoglobulin or T-cell receptor. An epitope includes a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. Epitopes can be contiguous or non-contiguous in relation to the primary structure of the antigen, for example, an NRP2 polypeptide. In particular embodiments, an epitope comprises, consists, or consists essentially of about, at least about, or no more than about 3, 4, 5, 6, 7, 8, 9, 10, 11, 12, 13, 14, 15, 16, 17, 18, 19, or 20 contiguous amino acids (i.e., a linear epitope) or non-contiguous amino acids (i.e., conformational epitope) of a reference sequence (see, e.g., Table N1. Table N2) or target molecule described herein.

An “epitope” includes that portion of an antigen or other macromolecule capable of forming a binding interaction that interacts with the variable region binding pocket of a binding protein. Such binding interaction can be manifested as an intermolecular contact with one or more amino acid residues of a CDR. Antigen binding can involve a CDR3 or a CDR3 pair. An epitope can be a linear peptide sequence (i.e., “continuous”) or can be composed of noncontiguous amino acid sequences (i.e., “conformational” or “discontinuous”). A binding protein can recognize one or more amino acid sequences; therefore an epitope can define more than one distinct amino acid sequence. Epitopes recognized by binding protein can be determined by peptide mapping and sequence analysis techniques well known to one of skill in the art. A “cryptic epitope” or a “cryptic binding site” is an epitope or binding site of a protein sequence that is not exposed or substantially protected from recognition within an unmodified polypeptide, but is capable of being recognized by a binding protein of a denatured or proteolyzed polypeptide. Amino acid sequences that are not exposed, or are only partially exposed, in the unmodified polypeptide structure are potential cryptic epitopes. If an epitope is not exposed, or only partially exposed, then it is likely that it is buried within the interior of the polypeptide. Candidate cryptic epitopes can be identified, for example, by examining the three-dimensional structure of an unmodified polypeptide.

The term “half maximal effective concentration” or “EC₅₀” refers to the concentration of an agent (e.g., antibody) as described herein at which it induces a response halfway between the baseline and maximum after some specified exposure time; the EC₅₀ of a graded dose response curve therefore represents the concentration of a compound at which 50% of its maximal effect is observed. EC50 also represents the plasma concentration required for obtaining 50% of a maximum effect in vivo. Similarly, the “EC₉₀” refers to the concentration of an agent or composition at which 90% of its maximal effect is observed. The “EC₉₀” can be calculated from the “EC50” and the Hill slope, or it can be determined from the data directly, using routine knowledge in the art. In some embodiments, the EC50 of an agent (e.g., antibody) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC₅₀ value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, efferocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent such as an antibody can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating.” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7, 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%. 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The term “migratory cells” refers to cells that are capable of movement from one place to another in response to a stimulus. Exemplary migratory cells include immune cells such as monocytes, Natural Killer (NK) cells, dendritic cells (immature or mature), subsets of dendritic cells including myeloid, plasmacytoid (also called lymphoid) and Langerhans cells, macrophages such as histiocytes, tissue resident macrophages such as Kupffer's cells, microglia cells in the CNS, alveolar macrophages, and peritoneal macrophages, macrophage subtypes such as M0, M1, Mox,M2a, M2b, and M2c macrophages, neutrophils, eosinophils, mast cells, basophils. B cells including plasma B cells, memory B cells, B-1 cells, and B-2 cells, CD45RO (naive T) cells, CD45RA (memory T) cells, CD4 Helper T Cells including Th1, Th2, and Tr1/Th3 cells, CD8 Cytotoxic T Cells, Regulatory T Cells, Gamma Delta T Cells, and thymocytes. Additional examples of migratory cells include fibroblasts, fibrocytes, tumor cells, and stem cells. The term “cell migration” refers to the movement of migratory cells, and the term “modulation of cell migration” refers to the modulation of the movement of any such migratory cells.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

“Expression control sequences” include regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, which have the ability to affect the transcription or translation, or subcellular, or cellular location of a coding sequence in a host cell. Exemplary expression control sequences are described in Goeddel; Gene Expression Technology: Methods in Enzymology 185, Academic Press. San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease Si) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types), and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include the Tet system. (U.S. Pat. Nos. 5,464,758 and 5,814,618), the Ecdysone inducible system (No et al., Proc. Natl. Acad. Sci. (1996) 93 (8): 3346-3351; the T-REx™ system (Invitrogen Carlsbad, CA), LacSwitch® (Stratagene, (San Diego, CA) and the Cre-ERT tamoxifen inducible recombinase system (Indra et al. Nuc. Acid. Res. (1999) 27 (22): 4324-4327; Nuc. Acid. Res. (2000) 28 (23): e99; U.S. Pat. No. 7,112,715; and Kramer & Fussenegger, Methods Mol. Biol. (2005) 308: 123-144) or any promoter known in the art suitable for expression in the desired cells.

An “expressible polynucleotide” includes a cDNA, RNA. mRNA or other polynucleotide that comprises at least one coding sequence and optionally at least one expression control sequence, for example, a transcriptional and/or translational regulatory element, and which can express an encoded polypeptide upon introduction into a cell, for example, a cell in a subject.

Various viral vectors that can be utilized to deliver an expressible polynucleotide include adenoviral vectors, herpes virus vectors, vaccinia virus vectors, adeno-associated virus (AAV) vectors, and retroviral vectors. In some instances, the retroviral vector is a derivative of a murine or avian retrovirus, or is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated. By inserting a polypeptide sequence of interest into the viral vector, along with another gene that encodes the ligand for a receptor on a specific target cell, for example, the vector may be made target specific. Retroviral vectors can be made target specific by inserting, for example, a polynucleotide encoding a protein. Illustrative targeting may be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector.

In particular embodiments, the expressible polynucleotide is a modified RNA or modified mRNA polynucleotide, for example, a non-naturally occurring RNA analog. In certain embodiments, the modified RNA or mRNA polypeptide comprises one or more modified or non-natural bases, for example, a nucleotide base other than adenine (A), guanine (G), cytosine (C), thymine (T), and/or uracil (U). In some embodiments, the modified mRNA comprises one or more modified or non-natural internucleotide linkages. Expressible RNA polynucleotides for delivering an encoded therapeutic polypeptide are described, for example, in Kormann et al., Nat Biotechnol. 29:154-7, 2011; and U.S. Application Nos. 2015/0111248; 2014/0243399; 2014/0147454; and 2013/0245104, which are incorporated by reference in their entireties.

The term “isolated” poly peptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., polypeptide such as an antibody) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

A “lipid nanoparticle” or “solid lipid nanoparticle” refers to one or more spherical nanoparticles with an average diameter of between about 10 to about 1000 nanometers, and which comprise a solid lipid core matrix that can solubilize lipophilic molecules. The lipid core is stabilized by surfactants (e.g., emulsifiers), and can comprise one or more of triglycerides (e.g., tristearin), diglycerides (e.g., glycerol behenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, which are incorporated by reference in their entireties. Certain compositions described herein are formulated with one or more lipid nanoparticles.

The terms or “neuropilin 2-associated disease” or “NRP2-associated disease” refer to diseases and conditions in which NRP2 activity, expression, and/or spatial distribution plays a role in the pathophysiology of that disease or condition. In some instances, NRP2-associated diseases are modulated by the anti-NRP2 antibodies of the present disclosure by altering the interaction of NRP2 with at least one NRP2 ligand, thereby impacting NRP2 activity, signaling, expression, and/or spatial distribution. In particular embodiments, the NRP2 is NRP2a variant 1 or variant 2, and the NRP2 ligand is CCL21 and/or CCR7. Thus, in certain embodiments, the NRP2-associated disease or condition is an “NRP2a-associated disease or condition”. Exemplary NRP2-associated diseases and conditions include without limitation, cancer and diseases or pathologies associated with cancer including cancer cell growth, cancer initiation, cancer migration, cancer cell adhesion, invasion, chemoresistance, and metastasis. Also included are diseases associated with inflammation and autoimmunity, and related inflammatory diseases, including disease associated with inappropriate immune cell activation or migration such as graft versus host disease (GVHD). Certain embodiments include diseases associated with lymphatic development, lymphangiogenesis, and lymphatic damage, including, for example, edema, lymphedema, secondary lymphedema, inappropriate fat absorption and deposition, excess fat deposition, and vascular permeability; diseases associated with infections, including latent infections; diseases associated with allergic disorders/diseases, allergic responses, including, for example, chronic obstructive pulmonary disorder (COPD), neutrophilic asthma, antineutrophil cytoplasmic antibody (ANCA)-associated systemic vasculitis, systemic lupus erythematosus, rheumatoid arthritis, inflammasome-related diseases, and skin-related neutrophil-mediated diseases such as pyoderma gangrenosum; diseases associated with granulomatous inflammatory diseases, including sarcoidosis and granulomas; diseases associated with fibrosis including fibrotic diseases, fibrosis, endothelial to mesenchymal transition (EMT), and wound healing; diseases associated with inappropriate smooth muscle contractility, smooth muscle compensation and decompensation, and inappropriate vascular smooth muscle cell migration and adhesion; diseases associated with inappropriate autophagy, phagocytosis, and efferocytosis; diseases associated with neuronal diseases, peripheral nervous system remodeling, and pain perception; diseases associated with bone development and bone remodeling. Typically, the term “inappropriate” refers to an activity or characteristic that associates with or causes a pathology or disease state.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the polypeptides (e.g., antibodies) described herein, and the polynucleotides that encode the same. “Variants” contain one or more substitutions, additions, deletions, and/or insertions relative to a reference polypeptide or polynucleotide (see. e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain the activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT. FASTA, and TFASTA in the Wisconsin Genetics Software Package Release 7.0, Genetics Computer Group, 575 Science Drive Madison. Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., antibody) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaPO₄). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”. “therapeutic dose,” “prophylactically effective amount,” or “diagnostically effective amount” is the amount of an agent (e.g., anti-NRP2a antibody, immunotherapy agent) needed to elicit the desired biological response following administration.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

### Anti-NRP2a Antibodies

Certain embodiments include antibodies, and antigen-binding fragments thereof, which bind to a human neuropilin 2a (NRP2a) polypeptide, specifically a human NRP2a variant 1 (v1) and/or variant 2 (v2) polypeptide. In particular embodiments, the antibodies, and antigen-binding fragments thereof, specifically bind to a human NRP2a v and v2 polypeptide, and also bind to a human NRP2a variant 3 (v3) polypeptide. In some embodiments, an antibody or antigen-binding fragment thereof selectively modulates (e.g., directly or indirectly interferes with, inhibits, reduces, stimulates, increases) binding of the human NRP2a v1 and/or v2 polypeptide to at least one NRP2a ligand, such as a plasma membrane receptor, growth factor, signaling molecule, integrin, plexin, or other ligand. Specific examples of NRP2a ligands include chemokine (C—C motif) ligand 21 (CCL21) polypeptide and/or a C—C chemokine receptor type 7 (CCR7) polypeptide.

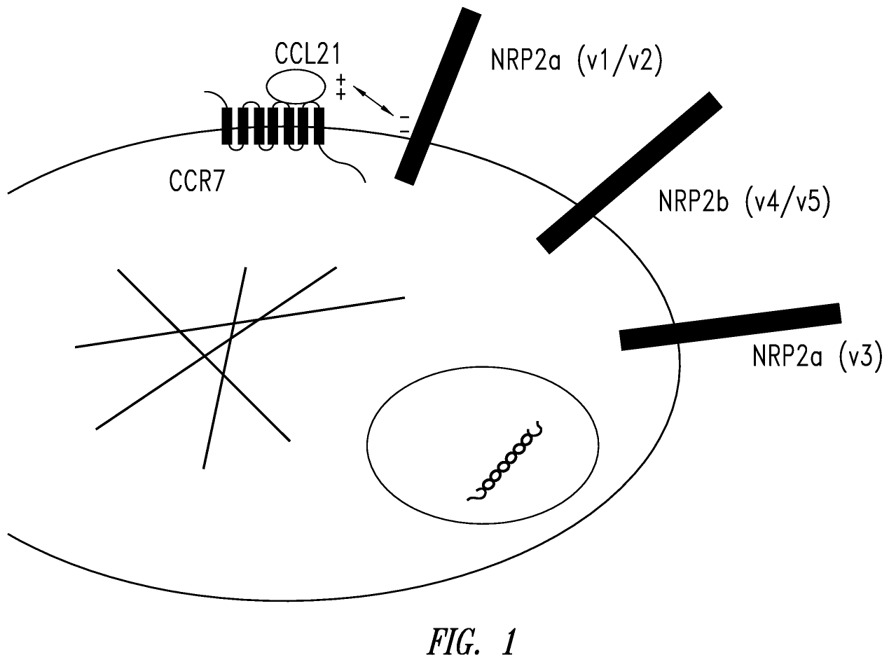

NRP2 is a single transmembrane receptor with a predominant extracellular region containing two CUB domains (a1/a2 combined domain), two Factor V/VIII homology domains (b1/b2 combined domain), a MAM domain (c domain) (see FIGS. 1A-1B), and a short juxtamembrane region that connects the c domain to the transmembrane domain (which traverses the plasma membrane). NRP2 is typically expressed in vivo as a mixture of various closely related splice variants, which are often grouped together as NRP2a, which comprises variants v1, v2 and v3, and NRP2b, which comprises variants v4 and v5. Variant v6 is a soluble form of NRP2 which is found in circulation.

The NRP2a and NRP2b splice variants have identical amino acid sequences over the a1, a2, b1, b2 and c domain, but differ in sequence over the juxtamembrane, transmembrane, and cytoplasmic regions. The NRP2a variants v1, v2, and v3 also differ in amino acid sequence over these regions based on their pattern of alternative splicing, with NRP2a v1 (931aa) and NRP2a v2 (926aa), having larger inserts compared to the relatively smaller NRP2a variant 3 (909aa). The different sizes of these alternatively spliced forms of NRP2a reflect a loss of a 5 amino acid stretch at the N-terminus of the juxtamembrane sequence from v1 to v2, then a further loss of 17 amino acids immediately C-terminal to the 5 amino acid deletion in the v3 variant. The C-terminal half of the juxtamembrane region, transmembrane helix, and cytoplasmic domain remains identical in all three NRP2a variants.

In both NRP2a and NRP2b, the a1a2 combined domain of NRP2 interacts with sema region of the semaphorins, and the b1 domain interacts with the semaphorin PSI and Ig-like domains. NRP2 has a higher affinity for SEMA3F and 3G; in contrast, SEMAs 3A, 3B and 3E preferentially interact with NRP1. Both NRP1 and NRP2 have similar affinity for SEMA 3C. The b1b2 combined domain of NRP2 interacts with several growth factors containing heparin-binding domains, including VEGF C & D, placenta growth factor (PIGF)-2, fibroblast growth factor (FGF), galectin, hepatocyte growth factor (HGF), platelet derived growth factor (PDGF), and transforming growth factor (TGF)-beta (see, for example, Prud'homme et al., Oncotarget. 3:921-939, 2012). NRP2 also interacts with various growth factor-specific receptors, and interactions with these receptors can occur independently of binding to SEMAs. In this context, integrins and growth factor receptors like VEGFR2 and VEGFR3, TGF-beta receptors, c-Met, EGFR, FGFR, PDGFR, have been shown to interact with NRPs and in general appear to increase the affinity of each ligand for its receptor and to modulate down stream signaling. The c domain (Mam) domain does not appear to be directly required for ligand binding, but may impact ligand specificity, receptor signaling, and NRP2 dimerization.

Neuropilin-2 modulates a broad range of cellular functions through its roles as an essential cell surface receptor and co-receptor for a variety of ligands (see, e.g., Guo and Vander Kooi, J. Cell. Biol. 290 No 49: 29120-29126, 2015). Additionally, recent data suggests that NRP2a and NRP2b are differentially expressed in normal tissues and in certain pathological conditions, suggesting that the relative expression of the NRP2 splice variants plays a key role in driving receptor cross-talk in a context dependent fashion in multiple NRP2 associated diseases. NRP2a and NRP2b also appear to play different and sometimes opposing roles in a broad range of cellular functions. Thus, the development of isoform-specific antibodies holds the promise of selectively modulating the activity of NRP2a variants and offers the opportunity to create a new generation of therapeutics with significantly enhanced cell type selectivity, isotype specificity, higher potency, and reduced toxicity compared to other anti-NRP2 antibodies which cannot discriminate between these isoforms.

Neuropilin-2 expression is associated with increased cellular plasticity, and epithelial to mesenchymal transition (EMT) in both normal and cancer cells, thereby increasing cellular survival and chemoresistance development during cancer treatment (see. e.g., Grandclement et al., PLoS ONE 6(7) e20444, 2011). Additionally. NRP2 expression is increased by TGF-beta exposure leading to both EMT and fibrosis development in fibroblasts and endothelial cells. (see, e.g., Pardali et al., Int. J. Mol. Sci. 18:2157, 2017). Accordingly, the development of NRP2a specific antibodies offers the possibility of selectively modulating cellular plasticity and survival, chemoresistance, and fibrosis development.

Neuropilin-2 expression promotes lymphangiogenesis (see, e.g., Doci et al., Cancer Res. 75:2937-2948, 2015) and single nucleotide polymorphisms (SNPs) in NRP2 are associated with lymphedema (see, e.g., Miaskowski et al., PLoS ONE 8(4) e60164, 2013). Accordingly, the development of NRP2a specific antibodies offers the possibility of selectively modulating the functions of these isoforms to regulate lymphangiogenesis and to treat lymphedema. NRP2 also regulates smooth muscle contractility and smooth muscle tone (see, e.g., Bielenberg et al., Amer. J. Path. 181:548-559, 2012). Accordingly, the development of NRP2a specific antibodies offers the possibility of selectively modulating the functions of these isoforms to regulate smooth muscle contractility, and muscle tone.

NRP2 directly contributes to cancer stem cell maintenance, and survival leading to increased tumor initiation, survival, chemo- and radio-resistance development, and metastasis (see, e.g., Goel et al., EMBO Mol. Med. 5:488-508, 2013; and Samuel et al., PLoS ONE 6(10) e23208, 2011), Prud'homme et al., Oncotarget 3:921-939, 2012). Accordingly, the development of NRP2a specific antibodies offers the possibility of selectively modulating the functions of these isoforms to regulate cancer stem cell growth, survival, chemo and radio-resistance development and metastasis.

Neuropilin-2 is expressed in various cells of the immune system, including lymphoid cells such as B and T cells, and myeloid cells such as basophils, eosinophil, monocytes, dendritic cells, NK cells, neutrophils, and macrophages, including tissue-specific macrophages, for example, alveolar macrophages. It is also expressed in endothelial and epithelial cells in the lung and other tissues, and in muscle cells (see, e.g., Bielenberg et al., Amer. J. Path. 181:548-559, 2012; Aung, et al., PLoS ONE 11(2) e0147358, 2016; Schellenburg et al., Mol. Imm 90:239-244, 2017; and Wild et al., Int. J. Exp. Path. 93:81-103, 2012). NRP2 regulates immune cell activation and migration (see, e.g., Mendes-da-Cruz et al., PLoS ONE 9(7) e103405, 2014). Accordingly, the development of NRP2a specific antibodies offers the possibility of selectively modulating the functions of these isoforms to regulate immune cell activation and migration, thereby providing or the development of anti-inflammatory, and immunomodulatory agents to inflammation and autoimmunity.

Neuropilin-2 also plays a key role in autophagy, endosome development, for example, by regulating late endosomal maturation, an important aspect of phagocytosis and efferocytosis, which respectively contribute to clearance of infections and apoptotic cells (see, e.g., Diaz-Vera et al., J. Cell. Sci. 130:697-711, 2017; Dutta et al., Cancer Res. 76:418-428, 2016). Accordingly, the development of NRP2a specific antibodies offers the possibility of selectively modulating the functions of these isoforms to regulate endosome development, phagocytosis, efferocytosis, and autophagy.

Neuropilin-2 is known to be a key player in the pathophysiology of many diseases (e.g., “NRP2-associated diseases”, as described herein) and interacts with a broad array of soluble ligands including semaphorin 3F, VEGF-C and D, and TGF-beta (see, for example, Table L1 and Table L2), and an array of cellular receptors and co-factors. NRP2 is also polysialated on dendritic cells, and actively interacts with the chemokine CCL21 and its receptor, CCR7 to mediate immune cell migration, and for which single nucleotide polymorphisms associated with ILD and RA have been described (see, e.g., Rey-Gallardo et al., Glycobiology 20:1139-1146, 2010; Stahl et al., Nat. Genet. 42:508-514, 2013; and Miller et al., Arthritis Rheum. 65:3239-3247). Additionally, soluble, circulating forms of NRP-2 are known (see, e.g., Parker et al., Structure 23(4) 677-687, 2015). Accordingly, given the central role played by NRP2 in pathophysiology in a broad range of diseases, it is evident that interactions between NRP2 and NRP2 ligand(s) (for example, NRP2 ligands from Table L1 and Table L2), and the modulation of those interactions with antibodies against NRP2 to selectively change the corresponding biological activities, provides broad potential for the treatment of diseases, including NRP2 associated diseases.

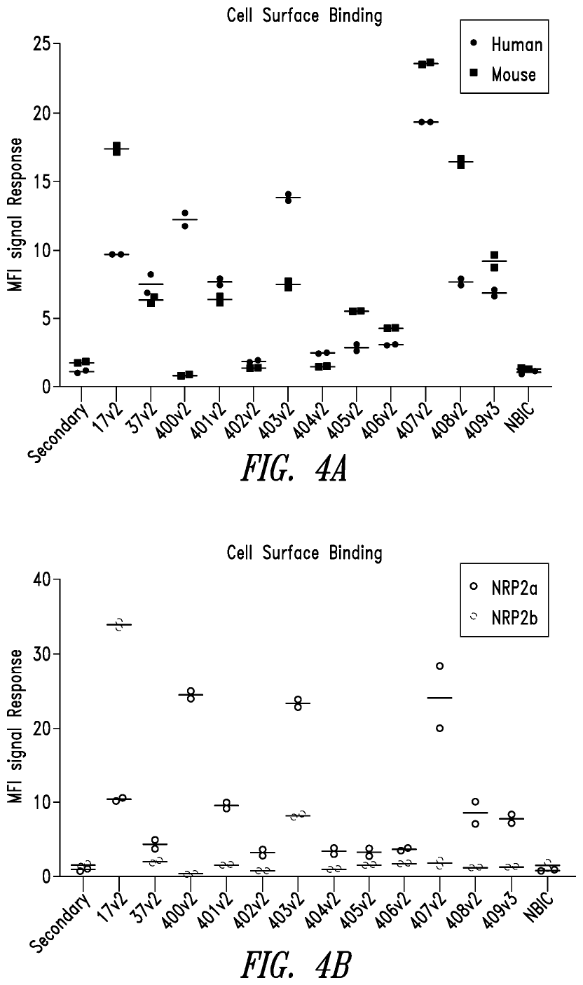

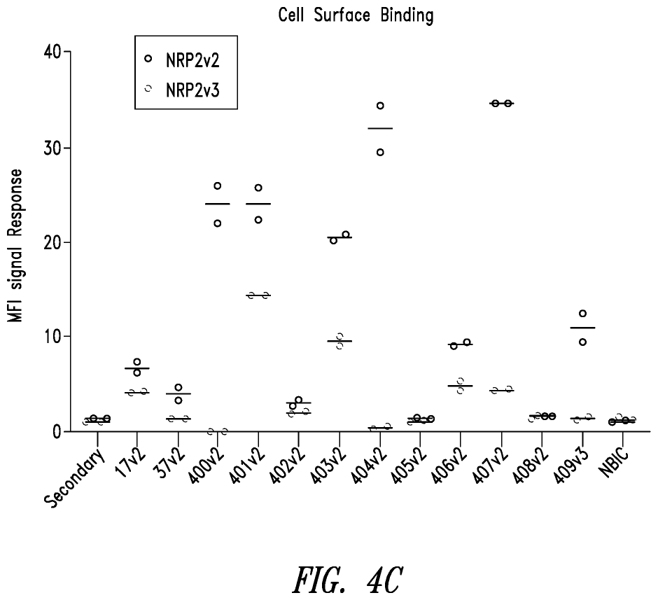

In particular embodiments, an antibody or antigen-binding fragment thereof selectively or preferentially binds to a human NRP2a v1 and/or v2 polypeptide relative to other NRP2 isoform polypeptides, including relative to a human neuropilin-2B (NRP2b) variant 4 (v4) polypeptide and/or a human NRP2b variant 5 (v5) polypeptide. That is, in some embodiments, an antibody or antigen-binding fragment thereof does not substantially bind to a human NRP2b v4 polypeptide or a NRP2b v5 polypeptide. Certain antibodies or antigen-binding fragments thereof also bind to a human NRP2a v3 polypeptide, and particular antibodies and antigen-binding fragments thereof do not substantially bind to a human NRP2a v3 polypeptide (see, e.g., FIG. 4C). Exemplary amino acid sequences of NRP2 polypeptides are provided in Table N1 below.

TABLE N1
Exemplary Human NRP2 Polypeptides

| | | | SEQ ID |
|---|---|---|---|
| Name | Residues | Sequence | NO: |
| Human NRP2a | 23-931 | QPDPPCGGRLNSKDAGYITSPGYPQDYPSHQNCEWIVYAP | 89 |
| variant 1 | | EPNQKIVINENPHFEIEKHDCKYDFIEIRDGDSESADLLG | |
| | | KHCGNIAPPTIISSGSMLYIKFTSDYARQGAGESLRYEIF | |
| | | KTGSEDCSKNFTSPNGTIESPGFPEKYPHNLDCTFTILAK | |
| | | PKMEIILQFLIFDLEHDPLQVGEGDCKYDWLDIWDGIPHV | |
| | | GPLIGKYCGTKTPSELRSSTGILSLTFHTDMAVAKDGFSA | |
| | | RYYLVHQEPLENFQCNVPLGMESGRIANEQISASSTYSDG | |
| | | RWTPQQSRLHGDDNGWTPNLDSNKEYLQVDLRFLTMLTAI | |
| | | ATQGAISRETQNGYYVKSYKLEVSTNGEDWMVYRHGKNHK | |
| | | VFQANNDATEVVLNKLHAPLLTREVRIRPQTWHSGIALRL | |
| | | ELFGCRVTDAPCSNMLGMLSGLIADSQISASSTQEYLWSP | |
| | | SAARLVSSRSGWFPRIPQAQPGEEWLQVDLGTPKTVKGVI | |
| | | IQGARGGDSITAVEARAFVRKFKVSYSINGKDWEYIQDPR | |
| | | TQQPKLFEGNMHYDTPDIRREDPIPAQYVRVYPERWSPAG | |
| | | IGMRLEVLGCDWTDSKPTVETLGPTVKSEETTTPYPTEEE | |
| | | ATECGENCSFEDDKDLQLPSGENCNEDFLEEPCGWMYDHA | |
| | | KWLRTTWASSSSPNDRTFPDDRNFLRLQSDSQREGQYARL | |
| | | ISPPVHLPRSPVCMEFQYQATGGRGVALQVVREASQESKL | |
| | | LWVIREDQGGEWKHGRIILPSYDMEYQIVFEGVIGKGRSG | |
| | | EIAIDDIRISTDVPLENCMEPISAFAGENFKVDIPEIHER | |
| | | EGYEDEIDDEYEVDWSNSSSATSGSGAPSTDKEKSWLYTL | |
| | | DPILITIIAMSSLGVLLGATCAGLLLYCTCSYSGLSSRSC | |
| | | TTLENYNFELYDGLKHKVKMNHQKCCSEA | |
| Human NRP2a | 23-926 | QPDPPCGGRLNSKDAGYITSPGYPQDYPSHQNCEWIVYAP | 90 |
| variant 2 | | EPNQKIVLNENPHFEIEKHDCKYDFIEIRDGDSESADLLG | |
| | | KHCGNIAPPTIISSGSMLYIKFTSDYARQGAGESLRYEIF | |
| | | KTGSEDCSKNFTSPNGTIESPGFPEKYPHNLDCTFTILAK | |
| | | PKMEIILQFLIFDLEHDPLQVGEGDCKYDWLDIWDGIPHV | |
| | | GPLIGKYCGTKTPSELRSSTGILSLTFHTDMAVAKDGFSA | |
| | | RYYLVHQEPLENFQCNVPLGMESGRIANEQISASSTYSDG | |
| | | RWTPQQSRLHGDDNGWTPNLDSNKEYLQVDLRFLTMLTAI | |
| | | ATQGAISRETQNGYYVKSYKLEVSTNGEDWMVYRHGKNHK | |
| | | VFQANNDATEVVLNKLHAPLLTRFVRIRPQTWHSGIALRL | |
| | | ELFGCRVTDAPCSNMLGMLSGLIADSQISASSTQEYLWSP | |
| | | SAARLVSSRSGWFPRIPQAQPGEEWLQVDLGTPKTVKGVI | |
| | | IQGARGGDSITAVEARAFVRKFKVSYSINGKDWEYIQDPR | |
| | | TQQPKLFEGNMHYDTPDIRREDPIPAQYVRVYPERWSPAG | |
| | | IGMRLEVLGCDWTDSKPTVETLGPTVKSEETTTPYPTEEE | |
| | | ATECGENCSFEDDKDLQLPSGENCNEDFLEEPCGWMYDHA | |
| | | KWLRTTWASSSSPNDRTFPDDRNFLRLQSDSQREGQYARL | |
| | | ISPPVHLPRSPVCMEFQYQATGGRGVALQVVREASQESKL | |
| | | LWVIREDQGGEWKHGRIILPSYDMEYQIVFEGVIGKGRSG | |
| | | EIAIDDIRISTDVPLENCMEPISAFAVDIPEIHEREGYED | |
| | | EIDDEYEVDWSNSSSATSGSGAPSTDKEKSWLYTLDPILI | |
| | | TIIAMSSLGVLLGATCAGLLLYCTCSYSGLSSRSCTTLEN | |
| | | YNFELYDGLKHKVKMNHQKCCSEA | |
| Human NRP2a | 23-909 | QPDPPCGGRLNSKDAGYITSPGYPQDYPSHQNCEWIVYAP | 91 |
| variant 3 | | EPNQKIVINENPHFEIEKHDCKYDFIEIRDGDSESADLLG | |
| | | KHCGNIAPPTIISSGSMLYIKFTSDYARQGAGESLRYEIF | |
| | | KTGSEDCSKNFTSPNGTIESPGFPEKYPHNLDCTFTILAK | |
| | | PKMEIILQFLIFDLEHDPLQVGEGDCKYDWLDIWDGIPHV | |
| | | GPLIGKYCGTKTPSELRSSTGILSLTFHTDMAVAKDGFSA | |
| | | RYYLVHQEPLENFQCNVPLGMESGRIANEQISASSTYSDG | |
| | | RWTPQQSRLHGDDNGWTPNLDSNKEYLQVDLRFLTMLTAI | |
| | | ATQGAISRETQNGYYVKSYKLEVSTNGEDWMVYRHGKNHK | |
| | | VFQANNDATEVVLNKLHAPLLTRFVRIRPQTWHSGIALRL | |
| | | ELFGCRVTDAPCSNMLGMLSGLIADSQISASSTQEYLWSP | |
| | | SAARLVSSRSGWFPRIPQAQPGEEWLQVDLGTPKTVKGVI | |
| | | IQGARGGDSITAVEARAFVRKFKVSYSINGKDWEYIQDPR | |
| | | TQQPKLFEGNMHYDTPDIRREDPIPAQYVRVYPERWSPAG | |
| | | IGMRLEVLGCDWTDSKPTVETLGPTVKSEETTTPYPTEEE | |
| | | ATECGENCSFEDDKDLQLPSGENCNEDFLEEPCGWMYDHA | |
| | | KWLRTTWASSSSPNDRTFPDDRNFLRLQSDSQREGQYARL | |
| | | ISPPVHLPRSPVCMEFQYQATGGRGVALQVVREASQESKL | |
| | | LWVIREDQGGEWKHGRIILPSYDMEYQIVFEGVIGKGRSG | |
| | | EIAIDDIRISTDVPLENCMEPISAFADEYEVDWSNSSSAT | |
| | | SGSGAPSTDKEKSWLYTLDPILITIIAMSSLGVLLGATCA | |
| | | GLLLYCTCSYSGLSSRSCTTLENYNFELYDGLKHKVKMNH | |
| | | QKCCSEA | |
| Human NRP2b | 23-906 | QPDPPCGGRLNSKDAGYITSPGYPQDYPSHQNCEWIVYAP | 92 |
| variant 4 | | EPNQKIVINENPHFEIEKHDCKYDFIEIRDGDSESADLLG | |
| | | KHCGNIAPPTIISSGSMLYIKFTSDYARQGAGESLRYEIF | |
| | | KTGSEDCSKNFTSPNGTIESPGFPEKYPHNLDCTFTILAK | |
| | | PKMEIILQFLIFDLEHDPLQVGEGDCKYDWLDIWDGIPHV | |
| | | GPLIGKYCGTKTPSELRSSTGILSLTFHTDMAVAKDGFSA | |
| | | RYYLVHQEPLENFQCNVPLGMESGRIANEQISASSTYSDG | |
| | | RWTPQQSRLHGDDNGWTPNLDSNKEYLQVDLRFLTMLTAI | |
| | | ATQGAISRETQNGYYVKSYKLEVSTNGEDWMVYRHGKNHK | |
| | | VFQANNDATEVVLNKLHAPLLTRFVRIRPQTWHSGIALRL | |
| | | ELFGCRVTDAPCSNMLGMLSGLIADSQISASSTQEYLWSP | |
| | | SAARLVSSRSGWFPRIPQAQPGEEWLQVDLGTPKTVKGVI | |
| | | IQGARGGDSITAVEARAFVRKFKVSYSINGKDWEYIQDPR | |
| | | TQQPKLFEGNMHYDTPDIRREDPIPAQYVRVYPERWSPAG | |
| | | IGMRLEVLGCDWTDSKPTVETLGPTVKSEETTTPYPTEEE | |
| | | ATECGENCSFEDDKDLQLPSGENCNEDFLEEPCGWMYDHA | |
| | | KWLRTTWASSSSPNDRTFPDDRNFLRLQSDSQREGQYARL | |
| | | ISPPVHLPRSPVCMEFQYQATGGRGVALQVVREASQESKL | |
| | | LWVIREDQGGEWKHGRIILPSYDMEYQIVFEGVIGKGRSG | |
| | | EIAIDDIRISTDVPLENCMEPISAFAGENEKGGTLLPGTE | |
| | | PTVDTVPMQPIPAYWYYVMAAGGAVLVLVSVALALVLHYH | |
| | | RFRYAAKKTDHSITYKTSHYTNGAPLAVEPTLTIKLEQDR | |
| | | GSHC | |
| Human NRP2b | 23-901 | QPDPPCGGRLNSKDAGYITSPGYPQDYPSHQNCEWIVYAP | 93 |
| variant 5 | | EPNQKIVINENPHFEIEKHDCKYDFIEIRDGDSESADLLG | |
| | | KHCGNIAPPTIISSGSMLYIKFTSDYARQGAGESLRYEIF | |
| | | KTGSEDCSKNFTSPNGTIESPGFPEKYPHNLDCTFTILAK | |
| | | PKMEIILQFLIFDLEHDPLQVGEGDCKYDWLDIWDGIPHV | |
| | | GPLIGKYCGTKTPSELRSSTGILSLTFHTDMAVAKDGFSA | |
| | | RYYLVHQEPLENFQCNVPLGMESGRIANEQISASSTYSDG | |
| | | RWTPQQSRLHGDDNGWTPNLDSNKEYLQVDLRFLTMLTAI | |
| | | ATQGAISRETQNGYYVKSYKLEVSTNGEDWMVYRHGKNHK | |
| | | VFQANNDATEVVLNKLHAPLLTRFVRIRPQTWHSGIALRL | |
| | | ELFGCRVTDAPCSNMLGMLSGLIADSQISASSTQEYLWSP | |
| | | SAARLVSSRSGWFPRIPQAQPGEEWLQVDLGTPKTVKGVI | |
| | | IQGARGGDSITAVEARAFVRKFKVSYSINGKDWEYIQDPR | |
| | | TQQPKLFEGNMHYDTPDIRREDPIPAQYVRVYPERWSPAG | |
| | | IGMRLEVLGCDWTDSKPTVETLGPTVKSEETTTPYPTEEE | |
| | | ATECGENCSFEDDKDLQLPSGENCNEDFLEEPCGWMYDHA | |
| | | KWLRTTWASSSSPNDRTFPDDRNFLRLQSDSQREGQYARL | |
| | | ISPPVHLPRSPVCMEFQYQATGGRGVALQVVREASQESKL | |
| | | LWVIREDQGGEWKHGRIILPSYDMEYQIVFEGVIGKGRSG | |
| | | EIAIDDIRISTDVPLENCMEPISAFAGGTLLPGTEPTVDT | |
| | | VPMQPIPAYWYYVMAAGGAVLVLVSVALALVLHYHRFRYA | |
| | | AKKTDHSITYKTSHYTNGAPLAVEPTLTIKLEQDRGSHC | |

Thus, in particular embodiments, an antibody or antigen-binding fragment thereof selectively or preferentially binds to a human NRP2a v1 and/or v2 poly-peptide from Table N1, and does not substantially bind to a human NRP2b v4 polypeptide, or a human NRP2b v5 poly peptide, for example, from Table N1. Certain antibodies or antigen-binding fragments thereof also bind to a human NRP2a v3 polypeptide from Table N1, and some antibodies and antigen-binding fragments thereof do not substantially bind to a human NRP2a v3 polypeptide from Table N1.

In particular embodiments, an antibody or antigen-binding fragment thereof binds to a human NRP2a v1 and/or 2 polypeptide at a unique epitope in the juxtamembrane domain, that is, an epitope that is present in human NRP2a v1 and/or v2 but is not present in human NRP2b v4 or NRP2b v5. In some instances, the unique epitope is present in human NRP2a v3, and in some instances, the unique epitope is not present in human NRP2a v3. Exemplary unique juxtamembrane epitopes are provided in Table N2 below.

TABLE N2
Exemplary juxtamembrane epitopes
of NRP2a vl and v2

| | | | SEQ ID |
|---|---|---|---|
| Name | Residues | Sequence | NO: |
| NRP2a | 803-864 | PISAFAGENEKVDIPEIH | 94 |
| Juxta- | | EREGYEDEIDDEYEVDWS | |
| membrane | | NSSSATSGSGAPSTDKEK | |
| Variant 1 | | SWLYTLDP | |
| NRP2a | 803-859 | PISAFAVDIPEIHEREGYE | 95 |
| Juxta- | | DEIDDEYEVDWSNSSSATS | |
| membrane | | GSGAPSTDKEKSWLYTLDP | |
| Variant 2 | | | |
| NRP2a | 803-832 | PISAFAVDIPEIHEREG | 96 |
| v1/v2 | | YEDEIDDEYEVDW | |
| unique | | | |
| epitope | | | |
| NRP2a | 810-832 | DIPEIHEREGYEDEIDDE | 97 |
| v1/v2 | | YEVDW | |
| unique | | | |
| epitope | | | |
| NRP2a | 803-820 | PISAFAVDIPEIHEREGY | 98 |
| v1/v2 | | | |
| unique | | | |
| epitope | | | |
| NRP2a | 810-820 | DIPEIHEREGY | 99 |
| v1/v2 | | | |
| unique | | | |
| epitope | | | |
| NRP2a | 818-832 | EGYEDEIDDEYEVDW | 100 |
| v1/v2 | | | |
| unique | | | |
| epitope | | | |
| NRP2a | 803-811 | PISAFAVDI | 101 |
| v1/v2 | | | |
| unique | | | |
| epitope | | | |
| NRP2a | 840-859 | SGSGAPSTDKEKSWLY | 102 |
| v1/v2 | | TLDP | |
| unique | | | |
| epitope | | | |
| NRP2a | 848-859 | DKEKSWLYTLDP | 103 |
| v1/v2 | | | |
| unique | | | |
| epitope | | | |
| NRP2a | 840-851 | SGSGAPSTDKEK | 104 |
| v1/v2 | | | |
| unique | | | |
| epitope | | | |

Thus, in some embodiments, an antibody or antigen-binding fragment thereof binds to the NRP2a v1 or v2 polypeptide at a sequence or epitope from Table N2. In some embodiments, the epitope comprises at least about 8, 9, 10, 11, or 12 contiguous amino acids of a sequence or epitope from Table N2, for example, any of SEQ ID NOs: 94-104, including combinations thereof. In some embodiments, an antibody or antigen-binding fragment thereof binds to continuous epitope, which comprises an epitope from Table N2. In some embodiments, an antibody or antigen-binding fragment thereof binds to a discontinuous epitope, which comprises one or more epitopes from Table N2. In specific embodiments, an antibody or antigen-binding fragment thereof binds to SEQ ID NO: 100, or about or at least about 8, 9, 10, 11, or 12 contiguous amino acids of SEQ ID NO: 100.

In some embodiments, an antibody or antigen-binding fragment thereof binds to an epitope in the neuropilin juxtamembrane domain of NRP2a v1, for example, residues 803-864, 813-864, 823-864, 833-864, 843-864, 853-864; 803-854, 803-844, 803-834, 803-824, and/or 803-814 as defined in SEQ ID NO: 94. In some embodiments, the at least one antibody or antigen-binding fragment thereof specifically binds to at least one epitope in the neuropilin juxtamembrane domain of NRP2a v2, for example, residues 803-859, 813-859, 823-859, 833-859, 843-859, 853-859; 803-849, 803-839, 803-829, 803-819, and/or 803-809 as defined in SEQ ID NO: 95. In some embodiments, an antibody or antigen-binding fragment thereof binds to at least one epitope in the neuropilin juxtamembrane domain of NRP2a v1/v2, for example, residues 818-832, 820-832, 822-832, 824-832, 826-832, 818-830, 818-828, 818-826, and/or 818-824 as defined in SEQ ID NO: 95.

In some embodiments, the at least one antibody or antigen-binding fragment thereof specifically binds to at least one epitope in the neuropilin juxtamembrane domain of NRP2a v1/v2, for example, residues 818-832, 820-832, 822-832, 824-832, 826-832, or 818-830, 818-828, 818-826, and/or 818-824 as defined in SEQ ID NO: 95. In specific embodiments, an antibody or antigen-binding fragment thereof binds to SEQ ID NO: 100.

In some embodiments, an antibody or antigen-binding fragment thereof binds to an NRP2a v1 and/or v2 poly peptide, and optionally a NRP2a v3 poly peptide, for example, a sequence from Table N1 and/or epitope from Table N2, with an affinity of about 10 pM to about 500 pM or to about 50 nM, or about, at least about, or no more than about 10, 20, 30, 40, 50, 60, 70, 80, 90, 100, 110, 120, 130, 140, 150, 160, 170, 180, 190, 200, 300, 400, 500, 600, 700, 800, 900 pM, 1 nM, 10 nM, 25 nM, or 50 nM, or optionally with an affinity that ranges from about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500) pM, about 30 pM to about 400 pM, about 30 pM to about 300 pM about 30 pM to about 200 pM about 30 pM to about 100 pM, about 30 pM to about 50 pM, or about 20 pM to about 200 pM, about 30 pM to about 300 pM, about 40 pM to about 4001 pM, about 50 pM to about 500 pM, about 60 pM to about 600 pM, about 70 pM to about 700 pM, about 80 pM to about 800 pM, about 90 pM to about 900 pM, about 100 pM to about 1 nM, about 1 nM to about 5 nM, about 5 nM to about 10 nM, about 10 nM to 25 nM, or about 25 nM to about 50 nM.

In some embodiments, the binding affinity of an antibody, or antigen-binding fragment thereof, for a human NRP2a v1 or v2 polypeptide (see Table N1) is at least about 1.5, 2, 4, 6, 8, 10, 20, 40, 60, 80, 100, 200, 400, 600, 800, or 1000 times stronger that its binding affinity for a NRP2a v3 polypeptide, a NRP2b v4 polypeptide, and/or a NRP2b v5 polypeptide (see Table N1).

In some embodiments, an antibody or antigen-binding fragment thereof specifically binds to at least one epitope within a region of a human NRP2a polypeptide that binds to or interacts with at least one “NRP2a ligand”, including any molecule that interacts with or binds reversibly to human NRP2a v1 and/or v2 but does not substantially interact with or bind to NRP2b v4 or NRP2b v5. General examples of NRP2 ligands are provided in Table L1.

TABLE L1
Exemplary Neuropilin Ligands

| Ligand | NRP1 | NRP2 |
|---|---|---|
| CCL21 | | + |
| CCR7 | | + |
| VEGF-A121 | + | |
| VEGF-A145 | | + |
| VEGF-A165 | + | + |
| VEGF-B167 | + | |
| VEGF-C | + | + |
| VEGF-D | + | + |
| VEGF-E | + | |
| PIGF-2 | + | + |
| VEGFR | +R1 and | +R1, R2, |
| | R2 | R3 |
| Heparin | + | + |
| Sema3A | + | |
| Sema3B, C, D, F, and G | + | + |
| Plexins Al, A2, | + | + |
| A3, A4, D1 | | |
| GIPC1, 2, and 3 | + | + |
| TGF-β1, β2, and | + | + |
| β3 receptors, | | |
| and LAP | | |
| TbRI and TbRII | + | + |
| FGF-1, 2, 4, and 7 | + | + |
| FGF receptor 1 | + | + |
| Hepatocyte growth factor | | + |
| receptors (c-Met) | | |
| Integrins (see Table N3) | + | + |
| Fibronectin | + | |
| Galectin-1 and Galectin | + | + |
| Receptors | | |
| Li-CAM | + | + |
| Glat-1 | + | |
| HRS polypeptides | + | |

Thus, in certain embodiments, the at least one NRP2a ligand is selected from Table L1. Specific examples of “NRP2a ligands” include a chemokine (C—C motif) ligand 21 (CCL21) polypeptide and a C—C chemokine receptor type 7 (CCR7) polypeptide. The amino acid sequences of exemplary NRP2a ligands are provided in Table L2 below.

TABLE L2
Exemplary NRP2a Ligands

| | | SEQ ID |
|---|---|---|
| Name | Sequence | NO: |
| Human | MAQSLALSLLILVLAFGIPRTQGSDGGAQDCCLKYSQRKIPAKVVRSYRKQEPSL | 105 |
| CCL21 | GCSIPAILFLPRKRSQAELCADPKELWVQQLMQHLDKTPSPQKPAQGCRKDRGAS | |
| Full- | KTGKKGKGSKGCKRTERSQTPKGP | |
| length | | |
| Human | SDGGAQDCCLKYSQRKIPAKVVRSYRKQEPSLGCSIPAILFLPRKRSQAELCADP | 106 |
| CCL21 | KELWVQQLMQHLDKTPSPQKPAQGCRKDRGASKTGKKGKGSKGCKRTERSQTPKG | |
| mature | P | |
| Human | MDLGKPMKSVLVVALLVIFQVCLCQDEVTDDYIGDNTTVDYTLFESLCSKKDVRN | 107 |
| CCR7 | FKAWFLPIMYSIICFVGLLGNGLVVLTYIYFKRLKTMTDTYLINLAVADILFLLT | |
| Full- | LPFWAYSAAKSWVFGVHECKLIFAIYKMSFFSGMLLLLCISIDRYVAIVQAVSAH | |
| length | RHRARVLLISKLSCVGIWILATVLSIPELLYSDLQRSSSEQAMRCSLITEHVEAF | |
| | ITIQVAQMVIGFLVPLLAMSFCYLVIIRTLLQARNFERNKAIKVIIAVVVVFIVE | |
| | QLPYNGVVLAQTVANENITSSTCELSKQLNIAYDVTYSLACVRCCVNPFLYAFIG | |
| | VKERNDLFKLFKDLGCLSQEQLRQWSSCRHIRRSSMSVEAETTTTESP | |
| Human | QDEVTDDYIGDNTTVDYTLFESLCSKKDVRNFKAWFLPIMYSIICFVGLLGNGLV | 108 |
| CCR7 | VLTYIYFKRLKTMTDTYLLNLAVADILFLLTLPFWAYSAAKSWVFGVHECKLIFA | |
| mature | TYKMSFFSGMLLLLCISIDRYVAIVQAVSAHRHRARVLLISKLSCVGIWILATVL | |
| | SIPELLYSDLQRSSSEQAMRCSLITEHVEAFITIQVAQMVIGFLVPLLAMSFCYL | |
| | VIIRTLLQARNFERNKAIKVIIAVVVVFIVEQLPYNGVVLAQTVANFNITSSTCE | |
| | LSKQLNIAYDVTYSLACVRCCVNPFLYAFIGVKERNDLFKLFKDLGCLSQEQLRQ | |
| | WSSCRHIRRSSMSVEAETTTTESP | |

Thus, in certain embodiments, the at least one NRP2a ligand is selected from Table L2, and the anti-NRP2a antibody or antigen-binding fragment thereof modulates (e.g., interferes with, inhibits, reduces) binding of a human NRP2a v1 and/or v2 polypeptide (for example, selected from Table N1) to an NRP2a ligand from Table L1 or Table L2, or a biologically-active fragment or variant thereof.

In some aspects, the at least one NRP2 ligand is selected from CCL21 and its receptor CCR7. CCR7 activity has been implicated in a diverse variety of disease states, including chronic inflammatory conditions (Moschovakis et al., 2012, Eur J Immunol. 42:1949-55), atherosclerosis (Luchtefeld et al., 2010, Circulation 122:1621-28), HIV infection (Evans et al., 2012. Cytokine Growth Factor Rev. 23:151-57) and cancer (Ben-Baruch, 2009, Cell Adhesion Migration 3:328-33). CCR7 activity is implicated in inflammatory disorders, including inflammatory bowel diseases (IBDs) such as Crohn's disease and ulcerative colitis, tissue or organ transplant rejection, asthma, allergic airway inflammation, airway smooth muscle hyperplasia, and fibrotic lung diseases (Gomperts et al., 2007, J Leukoc Biol. 82:449-56; Kawakami et al., 2012. Cell Immunol 2575:24-32; Saunders et al., 2009, Clin Exp Allergy 39:1684-92). CCR7 activity has also been implicated in rheumatoid arthritis (Moschovakis et al., 2012, Eur J Immunol. 42:1949-55). CCR7 activity has been implicated in multiple sclerosis (Aung et al., 2010, J Neuroimmunol. 226:158-64), psoriasis (Fan et al., 2008, Indian J Dermatol Venereol Leprol. 74(5):550; Bose et al., 2013, Am J Pathol, 183(2):413-421), and atherosclerosis (Luchtefeld et al., 2010, Circulation 10 122:1621-28). CCR7 activity has been implicated in HIV infection and other infections (Evans et al., 2012. Cytokine Growth Factor Rev. 23:151-57), including chronic and latent infections, including Leishmania donovani infection.

Various studies have revealed that CCR7 is expressed in a wide variety of tumour cells, including, for example, mantle cells lymphoma (MCL), follicular lymphoma, large B-cell lymphoma, AIDS-associated lymphoma, lymphoplasmacytic lymphoma, Burkitt lymphoma, B-cell acute lymphoblastic leukaemia, Hodgkin's disease, adult T-cell leukaemia/lymphoma, mycosis fungoides, blast crisis of chronic myeloproliferative syndromes, blast crisis of myelodysplastic syndromes, cancers such as breast cancer, non-small cell lung cancer, melanoma, gastric cancer or squamous cell carcinoma of the head and neck and colon carcinoma as B cell chronic lymphocytic leukemia, non-Hodgkin's lymphoma, breast cancer cell and malignant mammary tumor (see, for example, WO 2007/003426). CCR7 also plays a role in lymph node metastasis of various cancers (see, for example, Viola and Luster, 2008, Annu Rev Pharmacol Toxicol. 48:171-97).

Accordingly, anti-NRP2a antibodies which either directly modulate CCL21 binding, or CCR7 receptor, signaling would be expected find utility in modulating one or more of these diseases and disorders, including for the treatment of inflammatory disorders, cancer, tissue or organ transplant rejection, airway inflammation, RA, and for the treatment and prevention of latent and persistent infections.

In certain embodiments, an antibody or antigen-binding fragment thereof is a “blocking antibody”, which fully or substantially inhibits the binding between a human NRP2a v1 and/or v2 polypeptide and an NRP2a ligand such as human CCL21 and/or CCR7. In some embodiments, a “blocking antibody” inhibits about or at least about 80-100% (e.g., 80, 85, 90, 95, or 100%) of the theoretical maximal binding between the NRP2a v1 and/or v2 polypeptide and the NRP2a ligand after pre-incubation of the “blocking antibody” with the NRP2a polypeptide in a substantially stoichiometrically equivalent amount. As used herein, a “stoichiometrically equivalent amount” refers to a situation where the number of moles of one substance (e.g., anti-NRP2a antibody) is equivalent or substantially equivalent to the number of moles at least one other substance (e.g., NRP2a polypeptide) in a given equation or reaction.

In certain embodiments, an antibody or antigen-binding fragment thereof is a “partial-blocking antibody”, which at least partially but not fully inhibits the binding between a human NRP2a v1 and/or v2 polypeptide and an NRP2a ligand such as human CCL21 and/or CCR7. In some embodiments, a “partial-blocking antibody” inhibits about or at least about 20-80% (e.g., 20, 25, 30, 35, 40, 45, 50, 55, 60, 65, 70, 75, or 80%) of the theoretical maximal binding between the NRP2a poly peptide and the NRP2a ligand after pre-incubation of the “partial-blocking antibody” with the NRP2a polypeptide in a substantially stoichiometrically equivalent amount.

In specific embodiments, an antibody or antigen-binding fragment thereof inhibits, blocks, or otherwise reduces binding between the NRP2a v1 or v2 polypeptide and a human CCL21 polypeptide, for example, in an in vitro binding assay, an in vitro or ex vivo cell-based assay, or in vivo. In some embodiments, an antibody or antigen-binding fragment thereof antagonizes or reduces the theoretical maximal binding between a human NRP2a v1 and/or v2 polypeptide and a human CCL21 polypeptide by about or at least about 20-100% (e.g., about 20, 25, 30, 35, 40, 45, 50, 55, 60, 65, 70, 75, 80, 90, or 100%), for example, after pre-incubation of the anti-NRP2a antibody with the NRP2a poly peptide in a substantially stoichiometrically equivalent amount.

In some embodiments, an antibody or antigen-binding fragment thereof specifically inhibits, blocks, or otherwise reduces binding (e.g., dimerization) between the NRP2a v1 or v2 polypeptide and a human CCR7 polypeptide, for example, in an in vitro binding assay, an in vitro or ex vivo cell-based assay, or in vivo. In some embodiments, an antibody or antigen-binding fragment thereof antagonizes or reduces the theoretical maximal binding between a human NRP2a v1 and/or v2 polypeptide and human CCR7 polypeptide by about or at least about 20-100% (e.g., about 20, 25, 30, 35, 40, 45, 50, 55, 60, 65, 70, 75, 80, 90, or 100%), for example, after pre-incubation of the anti-NRP2a antibody with the NRP2a polypeptide in a substantially stoichiometrically equivalent amount.

In some embodiments, an antibody or antigen-binding fragment thereof modulates CCL21/CCR7-mediated signaling, for example, by about or at least about 20-100% (e.g., about 20, 25, 30, 35, 40, 45, 50, 55, 60, 65, 70, 75, 80, 90, or 100%) relative to control. Examples of CCL21/CCR7-mediated signaling activities include, without limitation, induction of immune cell migration, including dendritic cells or mature T-cells, inhibition of immature T-cells, and induction of tumor or cancer cell migration. Exemplary immune cells and tumor/cancer cells are described herein.

In some embodiments, an antibody or antigen-binding fragment thereof has an affinity (Kd or EC₅₀) for each of (i) a human NRP2a v1 and/or v2 polypeptide and (ii) the corresponding region of a cynomolgus monkey NRP2 polypeptide (see, for example, UniProt G7PL91), wherein the affinity for (i) and (ii) is within the range of about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, about 100 pM to about 1 nM, about 0.4 to about 1.2 nM, about 0.9 to about 5.5 nM, about 0.9 to about 5 nM, or about 1 nM to about 10 nM.

In some embodiments, an antibody or antigen-binding fragment thereof has an affinity (Kd or EC₅₀) for each of (i) a human NRP2a v1 and/or v2 polypeptide and (ii) the corresponding region of a murine NRP2 polypeptide, wherein the affinity for (i) and (ii) is within the range of about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, about 100 pM to about 1 nM, or about 1 nM to about 10 nM.

In certain embodiments, an antibody or antigen-binding fragment thereof binds selectively to a human NRP2a v1 and/or v2 polypeptide (see Table N1) relative to a corresponding murine NRP2 polypeptide, for instance, where its affinity for a human NRP2a v1 and/or v2 polypeptide is significantly stronger than its affinity for a corresponding murine NRP2 polypeptide, for example, by about or at least about 2, 5, 10, 20, 30, 40, 50, 100, 500, or 1,000-fold or more. In particular embodiments, an antibody or antigen-binding fragment thereof binds selectively to a human NRP2a v1 and/or v2 poly peptide and does not substantially bind to a corresponding murine NRP2 polypeptide. Certain exemplary murine NRP2 polypeptides include the Mus musculus NRP2 poly peptide (see, for example, UniProt 035375).

In certain embodiments, an antibody or antigen-binding fragment thereof is characterized by or comprises a heavy chain variable region (VH) sequence that comprises complementary determining region VHCDR1, VHCDR2, and VHCDR3 sequences, and a light chain variable region (VL) sequence that comprises complementary determining region VLCDR1, VLCDR2, and VLCDR3 sequences. Exemplary VH, VHCDR1, VHCDR2, VHCDR3, VL, VLCDR1, VLCDR2, and VLCDR3 sequences are provided in Table A1 and Table A2 below.

TABLE Al
Exemplary CDR Sequences

| | | |
|---|---|---|
| | | SEQ |
| Description | Sequence | ID NO: |

aNRP2-37v2

| | | |
|---|---|---|
| V_{H}CDR1 | GFTFSDYALS | 1 |
| V_{H}CDR2 | YISSGGDYIYYADTVRG | 2 |
| V_{H}CDR3 | GGQDDY | 3 |
| V_{L}CDR1 | RSSQSLVHSNGNTYLQ | 4 |
| V_{L}CDR2 | KVSNRFS | 5 |
| V_{L}CDR3 | SQSTHVPFT | 6 |

aNRP2-400v2

| | | |
|---|---|---|
| V_{H}CDR1 | GYTFRSYGIS | 7 |
| V_{H}CDR2 | EIYPRSGNTYYDENFKG | 8 |
| V_{H}CDR3 | SSITAVVAIPYYYAMDY | 9 |
| V_{L}CDR1 | SASQGISNYLN | 10 |
| V_{L}CDR2 | YTSSLHS | 11 |
| V_{L}CDR3 | QQYSKLPHT | 12 |

aNRP2-401v2

| | | |
|---|---|---|
| V_{H}CDR1 | GFTFSDYGMH | 13 |
| V_{H}CDR2 | TISRDINTVYYADTVKG | 14 |
| V_{H}CDR3 | GGYTDY | 15 |
| V_{L}CDR1 | RSSQSLVHSNGNTYLY | 16 |
| V_{L}CDR2 | KVSNRFS | 17 |
| V_{L}CDR3 | SQSTHVLT | 18 |

aNRP2-402v2

| | | |
|---|---|---|
| V_{H}CDR1 | GFTFSNYAMS | 19 |
| V_{H}CDR2 | SISDGGSYTYYPDNVKG | 20 |
| V_{H}CDR3 | DGGPREGYFDV | 21 |
| V_{L}CDR1 | RSSQSIVHSNGNTYLE | 22 |
| V_{L}CDR2 | KVSNRFS | 23 |
| V_{L}CDR3 | FQGSHVPYT | 24 |

aNRP2-403v2

| | | |
|---|---|---|
| V_{H}CDR1 | GYTFTSYWMH | 25 |
| V_{H}CDR2 | RIDPNSGDTKYNEKFKS | 26 |
| V_{H}CDR3 | SYDYALEY | 27 |
| V_{L}CDR1 | RSSQSLVHSNGNTYLH | 28 |
| V_{L}CDR2 | KVSNRFS | 29 |
| V_{L}CDR3 | SQNTRVPRT | 30 |

aNRP2-404v2

| | | |
|---|---|---|
| V_{H}CDR1 | GYTFTDYNMH | 31 |
| V_{H}CDR2 | YIYPYNGDSGYNQRFKS | 32 |
| V_{H}CDR3 | LGRGY | 33 |
| V_{L}CDR1 | KSSQSLLDSDGKTYLH | 34 |
| V_{L}CDR2 | LVSKLDS | 35 |
| V_{L}CDR3 | WQGTHFPWT | 36 |

aNRP2-405v2

| | | |
|---|---|---|
| V_{H}CDR1 | GFTFRRYAMS | 37 |
| V_{H}CDR2 | TITSGGSYTYYLDSVKG | 38 |
| V_{H}CDR3 | HGIYGGFDY | 39 |
| V_{L}CDR1 | RSSQSIVHSDGNTYLE | 40 |
| V_{L}CDR2 | KVSNRFS | 41 |
| V_{L}CDR3 | FQGSHVPFT | 42 |

aNRP2-406v2

| | | |
|---|---|---|
| V_{H}CDR1 | GYSFTGYFMN | 43 |
| V_{H}CDR2 | RINPYNGDTFYNQKFKG | 44 |
| V_{H}CDR3 | EVAEVPFDY | 45 |
| V_{L}CDR1 | KSSQSLLYRSNQKNYLA | 46 |
| V_{L}CDR2 | WASTRES | 47 |
| V_{L}CDR3 | QQYYSYPPT | 48 |

aNRP2-407v2

| | | |
|---|---|---|
| V_{H}CDR1 | GYSFTGYYMH | 49 |
| V_{H}CDR2 | RINPYNGATSYSQNFKD | 50 |
| V_{H}CDR3 | EETTAPFTY | 51 |
| V_{L}CDR1 | KSSQSLLYSSNQKNYLA | 52 |
| V_{L}CDR2 | WASTRES | 53 |
| V_{L}CDR3 | QHYYSYPPT | 54 |

aNRP2-408v2

| | | |
|---|---|---|
| V_{H}CDR1 | GFTFSSYAMS | 55 |
| V_{H}CDR2 | SISRGSITYYPDSVKG | 56 |
| V_{H}CDR3 | EYYYAMDY | 57 |
| V_{L}CDR1 | RASQDIGSRLN | 58 |
| V_{L}CDR2 | ATSSLDS | 59 |
| V_{L}CDR3 | LQYASSPYT | 60 |

aNRP2-409v3

| | | |
|---|---|---|
| V_{H}CDR1 | GFTFNTNAMN | 61 |
| V_{H}CDR2 | RIRTKSNNYATYYADSVKD | 62 |
| V_{H}CDR3 | LDSSGYVWFAY | 63 |
| V_{L}CDR1 | RASQDIGSRLN | 64 |
| V_{L}CDR2 | ATSSLDS | 65 |
| V_{L}CDR3 | LQYASSPYT | 66 |

aNRP2-401v5

| | | |
|---|---|---|
| V_{H}CDR1 | GFTFSDYGMH | 130 |
| V_{H}CDR2 | TISRDINTVYYADTVKG | 131 |
| V_{H}CDR3 | GGYTDY | 132 |
| V_{L}CDR1 | RSSQSLVHSNGNTYLY | 133 |
| V_{L}CDR2 | KVSNRFS | 134 |
| V_{L}CDR3 | SQSTHVLT | 135 |

aNRP2-401v6

| | | |
|---|---|---|
| V_{H}CDR1 | GFTFSDYGMH | 136 |
| V_{H}CDR2 | TISRDINTVYYADTVKG | 137 |
| V_{H}CDR3 | GGYTDY | 138 |
| V_{L}CDR1 | RSSQSLVHSNGNTYLY | 139 |
| V_{L}CDR2 | KVSNRFS | 140 |
| V_{L}CDR3 | AQSTHPLT | 141 |

aNRP2-401v7

| | | |
|---|---|---|
| V_{H}CDR1 | GFTFSDYGMH | 142 |
| V_{H}CDR2 | TISRSINTVYYADTVKG | 143 |
| V_{H}CDR3 | GGYTDY | 144 |
| V_{L}CDR1 | RSSQSLVHSNGNTYLY | 145 |
| V_{L}CDR2 | KVSNRFS | 146 |
| V_{L}CDR3 | AQSTHPLT | 147 |

aNRP2-401v8

| | | |
|---|---|---|
| V_{H}CDR1 | GFTFSDYGMH | 148 |
| V_{H}CDR2 | TISRDIQTVYYADTVKG | 149 |
| V_{H}CDR3 | GGYTDY | 150 |
| V_{L}CDR1 | RSSQSLVHSNGNTYLY | 151 |
| V_{L}CDR2 | KVSNRFS | 152 |
| V_{L}CDR3 | AQSTHPLT | 153 |

aNRP2-402v9

| | | |
|---|---|---|
| V_{H}CDR1 | GFTFSNYAMS | 154 |
| V_{H}CDR2 | SISDGGSYTYYPDNVKG | 155 |
| V_{H}CDR3 | DGGPREGYFDV | 156 |
| V_{L}CDR1 | RSSQSIVHSNGNTYLE | 157 |
| V_{L}CDR2 | KVSNRFS | 158 |
| V_{L}CDR3 | FQGSHVPYT | 159 |

aNRP2-401 Variant Consensus Sequences

| | | |
|---|---|---|
| V_{H}CDR1 | GFTFSDYGMH | 13 |
| V_{H}CDR2 | X₁₄X₁₅X₁₆X₁₇X₁₈X₁₉X₂₀X₂₁X₂₂X₂₃X₂₄ADTVKG | 127 |
| V_{H}CDR3 | GX₁X₂X₃X₄X₅ | — |
| V_{L}CDR1 | RSSQSLVHSNGNTYLY | 16 |
| V_{L}CDR2 | KVSNRFS | 17 |
| V_{L}CDR3 | X₆X₇X₈X₉X₁₀X₁₁X₁₂X₁₃ | — |

See Table E7 for definition of ″X″ residues

TABLE A2
Exemplary Polypeptide Sequences

| | | |
|---|---|---|
| | | SEQ ID |
| Description | Sequence | NO; |

aNRP2-37v2

| | | |
|---|---|---|
| Heavy chain | DVKLVESGEGLVKPGGSLKLSCAASGFTFSDYALSWVRQTPEKRLEWV | 67 |
| variable | AYISSGGDYIYYADTVRGRFTISRDNARNTLYLQMSSLQSEDTAIYYC | |
| region (V_{H}) | TRGGQDDYWGQGTTLTVSS | |
| Light chain | DVVMTQTPLSLPVILGDQASISCRSSQSLVHSNGNTYLQWYLQKPGQS | 68 |
| variable | PKLLIYKVSNRFSGVPDRFSGSGSGTDFTLKISRVEAEDLGVYFCSQS | |
| region (V_{L}) | THVPFTFGSGTNLEIK | |

aNRP2-400v2

| | | |
|---|---|---|
| Heavy chain | EVQLQESGAELARPGASVKLSCKASGYTFRSYGISWVKQRTGQGLEWI | 69 |
| variable | GEIYPRSGNTYYDENFKGRATLTADKSSSTAFMELRSLTSEDSAVYFC | |
| region (V_{H}) | ARSSITAVVAIPYYYAMDYWGQGTSVTVSS | |
| Light chain | DIQMTQTTSSLSASLGDRVTISCSASQGISNYLNWYQQKPDGSVKLLI | 70 |
| variable | YYTSSLHSGVPSREFGSGSGTDYSLTISNLEPEDIATYYCQQYSKLPH | |
| region (V_{L}) | TFGGGTKLELK | |

aNRP2-401v2

| | | |
|---|---|---|
| Heavy chain | EVQLQESGGGLVKPGGSLKLSCAASGFTFSDYGMHWVRQAPEKGLEWV | 71 |
| variable | ATISRDINTVYYADTVKGRFTISRDNAKNTLFLQVTSLRSEDTAMYYC | |
| region (V_{H}) | TRGGYTDYWGQGTTLTVSS | |
| Light chain | DIVMTQTPLSLPVSLGDQASISCRSSQSLVHSNGNTYLYWYLQRPGQS | 72 |
| variable | PKLLIYKVSNRFSGVPDRFSGSGSGTDFTLKISRVEAEDLGVYFCSQS | |
| region (V_{L}) | THVLTFGGGTKLEIK | |

aNRP2-402v2

| | | |
|---|---|---|
| Heavy chain | EVQLVESGGDLVKPGGSLKLSCAASGFTFSNYAMSWVRQTPEKRLEWV | 73 |
| variable | ASISDGGSYTYYPDNVKGRFTISRDSAKKSLYLQMSHLKSEDTAMYYC | |
| region (V_{H}) | TKDGGPREGYFDVWGTGTTVTVSS | |
| Light chain | DVLMTQTPLSLPVSLGDQASISCRSSQSIVHSNGNTYLEWYLQKPGQS | 74 |
| variable | PKLLIYKVSNRFSGVPDRFSGSGSGTDFTLKISRVEAEDLGVYYCFQG | |
| region (V_{L}) | SHVPYTFGGGTKLEIK | |

aNRP2-403v2

| | | |
|---|---|---|
| Heavy chain | QVQLQQPGAELVKPGASVKLSCKASGYTFTSYWMHWVKQRPGRGLEWI | 75 |
| variable | GRIDPNSGDTKYNEKFKSKATLTVDKPSSTAYMQVSSLTSEDSAVYYC | |
| region (V_{H}) | ARSYDYALEYWGQGTSVTVSS | |
| Light chain | DVVMTQTPLSLPVSLGDQASISCRSSQSLVHSNGNTYLHWFLQKPGQS | 76 |
| variable | PNLLIYKVSNRFSGVPDRFSGSGSGTDFTLKISRVEAEDLGVYFCSQN | |
| region (V_{L}) | TRVPRTFGGGTKLEIK | |

aNRP2-404v2

| | | |
|---|---|---|
| Heavy chain | EVQLQQSGPELVKPGASVKISCKASGYTFTDYNMHWVKQSHGKSLEWI | 77 |
| variable | GYIYPYNGDSGYNQRFKSEATLTVDISSSTAYMELRSLTSDDSAVYYC | |
| region (V_{H}) | ARLGRGYWGQGTTLTVSS | |
| Light chain | DVVMTQTPLTLSVTVGQPASISCKSSQSLLDSDGKTYLHWLFQRPGQS | 78 |
| variable | PRRLIYLVSKLDSGVPDRFTGSGSGTDFTLKISRVEAEDLGVYYCWQG | |
| region (V_{L}) | THEPWTFGGGTKLEIK | |

aNRP2-405v2

| | | |
|---|---|---|
| Heavy chain | EVMLVESGGGLVKPGGSLKLSCAASGFTFRRYAMSWVRQTPEKGLEWV | 79 |
| variable | ATITSGGSYTYYLDSVKGRITISRDNAKNTLYLQMSSLRSEDTAMYYC | |
| region (V_{H}) | ARHGIYGGFDYWGQGTTLTVSS | |
| Light chain | DVLMTQTPLSLPVSLGDQASISCRSSQSIVHSDGNTYLEWYLQKPGQS | 80 |
| variable | PNLLIYKVSNRFSGVPDRFSGSGSGTDFTLKISRVEAEDLGIYYCFQG | |
| region (V_{L}) | SHVPFTFGSGTKLEIK | |

aNRP2-406v2

| | | |
|---|---|---|
| Heavy chain | EVQLQQSGPELVKPGASVKISCKASGYSFTGYFMNWVKQSHGKSLEWI | 81 |
| variable | GRINPYNGDTFYNQKFKGKATLTVDKSSSTAHMALLSLTSEDSAVYYC | |
| region (V_{H}) | GREVAEVPFDYWGQGTTLTVSS | |
| Light chain | DIVMSQSPSSLAVSVGEKVTMSCKSSQSLLYRSNQKNYLAWYQQKPGQ | 82 |
| variable | SPKLLIYWASTRESGVPDRFTGSGSGTDFTLTISSVKAEDLAVYYCQQ | |
| region (V_{L}) | YYSYPPTFGAGTKLELK | |

aNRP2-407v2

| | | |
|---|---|---|
| Heavy chain | EVQLQQSGPELVKPGASVTISCKASGYSFTGYYMHWVKQSHVKSLEWI | 83 |
| variable | GRINPYNGATSYSQNFKDKASLTIDKSSSTAYMELHSLTSEDSAVYYC | |
| region (V_{H}) | AREETTAPFTYWGQGTLVTVSA | |
| Light chain | DIVMSQSPSSLAVSVGEKVTMSCKSSQSLLYSSNQKNYLAWYQQKPGQ | 84 |
| variable | SPKLLIYWASTRESGVPDRFTGSGSGTDFTLTISSVQAEDLAVYFCQH | |
| region (V_{L}) | YYSYPPTFGGGTKLEIK | |

aNRP2-408v2

| | | |
|---|---|---|
| Heavy chain | EVKLVESGGGLVKPGGSLKLSCAASGFTFSSYAMSWVRQTPEKRLEWV | 85 |
| variable | ASISRGSITYYPDSVKGRFTISRDNAGNILYLQMSSLRSEDTAMYYCA | |
| region (V_{H}) | REYYYAMDYWGQGTSVTVSS | |
| Light chain | DIQMTQSPSSLSASLGERVSLTCRASQDIGSRLNWLQQEPDGTIKRLI | 86 |
| variable | YATSSLDSGVPKRFSGSRSGSDYSLTISSLESEDEVDYYCLQYASSPY | |
| region (V_{L}) | TFGGGTKLEIK | |

aNRP2-409v3

| | | |
|---|---|---|
| Heavy chain | EVQFVETGGGLVQPKGSLKLSCAASGFTFNTNAMNWVRQAPGKGLEWV | 87 |
| variable | ARIRTKSNNYATYYADSVKDRFTISRDDSQNILYLQMNNLKTEDTAMY | |
| region (V_{H}) | YCVTLDSSGYVWFAYWGQGTLVTVSA | |
| Light chain | DIQMTQSPSSLSASLGERVSLTCRASQDIGSRLNWLQQEPDGTIKRLI | 88 |
| variable | YATSSLDSGVPKRFSGSRSGSDYSLTISSLESEDFVDYYCLQYASSPY | |
| region (V_{L}) | TFGGGTKLEIK | |

aNRP2-401v5

| | | |
|---|---|---|
| Heavy chain | EVQLVESGGGLVQPGGSLRLSCAASGFTFSDYGMHWVRQAPGKGLEWV | 160 |
| variable | STISRDINTVYYADTVKGRFTISRDNAKNSLYLQMNSLRAEDTAVYYC | |
| region (V_{H}) | ARGGYTDYWGQGTTVTVSS | |
| Light chain | DIVMTQTPLSLSVTPGQPASISCRSSQSLVHSNGNTYLYWYLQKPGQS | 161 |
| variable | PQLLIYKVSNRFSGVPDRFSGSGSGTDETLKISRVEAEDVGVYYCSQS | |
| region (V_{L}) | THVLTFGGGTKVEIK | |

aNRP2-401v6

| | | |
|---|---|---|
| Heavy chain | EVQLVESGGGLVQPGGSLRLSCAASGFTFSDYGMHWVRQAPGKGLEWV | 162 |
| variable | STISRDINTVYYADTVKGRFTISRDNAKNSLYLQMNSLRAEDTAVYYC | |
| region (V_{H}) | ARGGYTDYWGQGTTVTVSS | |
| Light chain | DIVMTQTPLSLSVTPGQPASISCRSSQSLVHSNGNTYLYWYLQKPGQS | 163 |
| variable | PQLLIYKVSNRFSGVPDRFSGSGSGTDETLKISRVEAEDVGVYYCAQS | |
| region (V_{L}) | THPLTFGGGTKVEIK | |

aNRP2-401v7

| | | |
|---|---|---|
| Heavy chain | EVQLVESGGGLVQPGGSLRLSCAASGFTFSDYGMHWVRQAPGKGLEWV | 164 |
| variable | STISRSINTVYYADTVKGRFTISRDNAKNSLYLQMNSLRAEDTAVYYC | |
| region (V_{H}) | ARGGYTDYWGQGTTVTVSS | |
| Light chain | DIVMTQTPLSLSVTPGQPASISCRSSQSLVHSNGNTYLYWYLQKPGQS | 165 |
| variable | PQLLIYKVSNRFSGVPDRFSGSGSGTDFTLKISRVEAEDVGVYYCAQS | |
| region (V_{L}) | THPLTFGGGTKVEIK | |

aNRP2-401v8

| | | |
|---|---|---|
| Heavy chain | EVQLVESGGGLVQPGGSLRLSCAASGFTFSDYGMHWVRQAPGKGLEWV | 166 |
| variable | STISRDIQTVYYADTVKGRFTISRDNAKNSLYLQMNSLRAEDTAVYYC | |
| region (V_{H}) | ARGGYTDYWGQGTTVTVSS | |
| Light chain | DIVMTQTPLSLSVTPGQPASISCRSSQSLVHSNGNTYLYWYLQKPGQS | 167 |
| variable | PQLLIYKVSNRFSGVPDRFSGSGSGTDFTLKISRVEAEDVGVYYCAQS | |
| region (V_{L}) | THPLTFGGGTKVEIK | |

aNRP2-402v9

| | | |
|---|---|---|
| Heavy chain | EVQLVESGGGLVKPGGSLRLSCAASGFTFSNYAMSWVRQAPGKGLEWV | 168 |
| variable | SSISDGGSYTYYPDNVKGRFTISRDNAKNSLYLQMNSLRAEDTAVYYC | |
| region (V_{H}) | ARDGGPREGYFDVWGKGTTVTVSS | |
| Light chain | DVVMTQSPLSLPVTLGQPASISCRSSQSIVHSNGNTYLEWYQQRPGQS | 169 |
| variable | PRLLIYKVSNRFSGVPDRFSGSGSGTDFTLKISRVEAEDVGVYYCFQG | |
| region (V_{L}) | SHVPYTFGGGTKVEIK | |

aNRP2-401 Humanized Variant Consensus Sequences

| | | |
|---|---|---|
| Heavy chain | EVQLVESGGGLVQPGGSLRLSCAASGFTFSDYGMHWVRQAPGKGLEWV | 170 |
| variable | SX₁X₁₅X₁₆X₁₇X₁₈X₁₉X₂₀X₂₁X₂₂X₂₃X₂₄ADTVKGRFTISRDNAKNSLYL | |
| region (V_{H}) | QMNSLRAEDTAVYYCARGX:X₂X₃X₄X₅WGQGTTVTVSS | |
| Light chain | DIVMTQTPLSLSVTPGQPASISCRSSQSLVHSNGNTYLYWYLQKPGQS | 171 |
| variable | PQLLIYKVSNRFSGVPDRFSGSGSGTDFTLKISRVEAEDVGVYYCXX | |
| region (V_{L}) | ₇X₈X₉X₁₀X₁X₁₂X₁₃FGGGTKVEIK | |

See Table E7 for definition of “X” residues

Thus, in certain embodiments, an antibody or antigen-binding fragment thereof comprises
a heavy chain variable region (VH) sequence that comprises complementary determining region VHCDR1, VHCDR2, and VHCDR3 sequences selected from Table A1 and variants thereof which specifically bind to a human NRP2a poly peptide or epitope thereof (selected, for example, from Table N1, Table N2); and
a light chain variable region (VL) sequence that comprises complementary determining region VLCDR1, VLCDR2, and VLCDR3 sequences selected from Table A1 and variants thereof which specifically bind to the human NRP2 polypeptide or epitope thereof (selected, for example, from Table N1, Table N2).

In certain embodiments, the CDR sequences are as follows:
the VHCDR1, VHCDR2, and VHCDR3 sequences comprise SEQ ID NOs: 130-132, respectively, and the VLCDR1, VLCDR2, and VLCDR3 sequences comprise SEQ ID NOs: 133-135, respectively, including variants thereof having 1, 2, 3, 4, 5, or 6 total alterations across all of the CDR regions;
the VHCDR1, VHCDR2, and VHCDR3 sequences comprise SEQ ID NOs: 136-138, respectively, and the VLCDR1, VLCDR2, and VLCDR3 sequences comprise SEQ ID NOs: 139-141, respectively, including variants thereof having 1, 2, 3, 4, 5, or 6 total alterations across all of the CDR regions;
the VHCDR1, VHCDR2, and VHCDR3 sequences comprise SEQ ID NOs: 142-144, respectively, and the VLCDR1, VLCDR2, and VLCDR3 sequences comprise SEQ ID NOs: 145-147, respectively, including variants thereof having 1, 2, 3, 4, 5, or 6 total alterations across all of the CDR regions;
the VHCDR1, VHCDR2, and VHCDR3 sequences comprise SEQ ID NOs: 148-150, respectively, and the VLCDR1, VLCDR2, and VLCDR3 sequences comprise SEQ ID NOs: 151-153, respectively, including variants thereof having 1, 2, 3, 4, 5, or 6 total alterations across all of the CDR regions;
the VHCDR1, VHCDR2, and VHCDR3 sequences comprise SEQ ID NOs: 154-156, respectively, and the VLCDR1, VLCDR2, and VLCDR3 sequences comprise SEQ ID NOs: 157-159, respectively, including variants thereof having 1, 2, 3, 4, 5, or 6 total alterations across all of the CDR regions;
the VHCDR1, VHCDR2, and VHCDR3 sequences comprise SEQ ID NOs: 1-3, respectively, and the VLCDR1, VLCDR2, and VLCDR3 sequences comprise SEQ ID NOs: 4-6, respectively, including variants thereof;
the VHCDR1, VHCDR2, and VHCDR3 sequences comprise SEQ ID NOs: 7-9, respectively, and the VLCDR1, VLCDR2, and VLCDR3 sequences comprise SEQ ID NOs: 10-12, respectively, including variants thereof;
the VHCDR1, VHCDR2, and VHCDR3 sequences comprise SEQ ID NOs: 13-15 respectively, and the VLCDR1, VLCDR2, and VLCDR3 sequences comprise SEQ ID NOs: 16-18, respectively, including variants thereof;
the VHCDR1, VHCDR2, and VHCDR3 sequences comprise SEQ ID NOs: 19-21, respectively, and the VLCDR1, VLCDR2, and VLCDR3 sequences comprise SEQ ID NOs: 22-24, respectively, including variants thereof;
the VHCDR1, VHCDR2, and VHCDR3 sequences comprise SEQ ID NOs: 25-27, respectively, and the VLCDR1, VLCDR2, and VLCDR3 sequences comprise SEQ ID NOs: 28-30, respectively, including variants thereof;
the VHCDR1, VHCDR2, and VHCDR3 sequences comprise SEQ ID NOs: 31-33, respectively, and the VLCDR1, VLCDR2, and VLCDR3 sequences comprise SEQ ID NOs: 34-36, respectively, including variants thereof;
the VHCDR1, VHCDR2, and VHCDR3 sequences comprise SEQ ID NOs: 37-39, respectively, and the VLCDR1, VLCDR2, and VLCDR3 sequences comprise SEQ ID NOs: 40-42, respectively, including variants thereof;
the VHCDR1, VHCDR2, and VHCDR3 sequences comprise SEQ ID NOs: 43-45, respectively, and the VLCDR1, VLCDR2, and VLCDR3 sequences comprise SEQ ID NOs: 46-48, respectively, including variants thereof;
the VHCDR1, VHCDR2, and VHCDR3 sequences comprise SEQ ID NOs: 49-51, respectively, and the VLCDR1, VLCDR2, and VLCDR3 sequences comprise SEQ ID NOs: 52-54, respectively, including variants thereof;
the VHCDR1, VHCDR2, and VHCDR3 sequences comprise SEQ ID NOs: 55-57, respectively, and the VLCDR1, VLCDR2, and VLCDR3 sequences comprise SEQ ID NOs: 58-60, respectively, including variants thereof; or the VHCDR1, VHCDR2, and VHCDR3 sequences comprise SEQ ID NOs: 61-63, respectively, and the VLCDR1, VLCDR2, and VLCDR3 sequences comprise SEQ ID NOs: 64-66, respectively, including variants thereof.

In certain embodiments, an antibody or antigen-binding fragment thereof (for example, a variant of a 401 antibody or antigen-binding fragment thereof) comprises CDR consensus sequences, for example, wherein the VHCDR1, VHCDR2, and VHCDR3 sequences comprise SEQ ID NOs: 13, 127, and GX₁X₂X₃X₄X₅ (wherein X, is G, A, or S, X₂ is Y, F, K, L, or R, X₃ is T, A, G, I, L, Q, or V, X₄ is D, A, G, K, N, Q, R, or S, and X₅ is Y, A, D, E, F, G, H, I, K, L, N, Q, R, S, T, or V), respectively, and the VLCDR1, VLCDR2, and VLCDR3 sequences comprise SEQ ID NOs: 16, 17, and X₆X₇X₈X₉X₁₀X₁₁X₁₂X₁₃ (wherein X_{F} is S, A, G, I, L, P, T, or V, X₇ is Q, A, G, R, or S, X₈ is S, A, H, K, L, Q, or T, X₉ is T, F, G, H, I, K, L, N, Q, R, S, V, or Y, X₁₀ is H, A, D, E, F, G, I, K, L, N, Q, R, S, T, or Y, X₁₁ is V, A, E, F, G, H, I, K, L, N, P, Q, R, S, T, or Y, X₁₂ is L, A, E, H, I, N, P, Q, S, T, or V, and X₁₃ is T, A, D, E, F, G, I, K, L, N, Q, R, S, or V), respectively (see Table E7 for definition of “X” residues).

Also included are variants thereof, including affinity matured variants, which bind to a human NRP2a polypeptide or epitope thereof (see, for example, Table N1, Table N2), for example, variants having 1, 2, 3, 4, 5, or 6 total alterations across all of the CDR regions, for example, one or more the V_{H}CDR1, V_{H}CDR2, V_{H}CDR3, V_{L}CDR1, V_{L}CDR2, and/or V_{L}CDR3 sequences described herein. Exemplary “alterations” include amino acid substitutions, additions, and deletions.

In certain embodiments, the V_{H} sequence is at least 80, 85, 90, 95, 97, 98, 99, or 100% identical to a sequence selected from Table A2, including, for example, wherein the V_{H} sequence has 1, 2, 3, 4, or 5 alterations in one or more framework regions.

In some embodiments, the V_{L} sequence is at least 80, 85, 90, 95, 97, 98, 99, or 100% identical to a sequence selected from Table A2, including, for example, wherein the V_{L} sequence has 1, 2, 3, 4, or 5 alterations in one or more framework regions.

In some embodiments, the V_{H} and V_{L} sequences of an antibody or antigen-binding fragment are as follows:
the V_{H} sequence is at least 80, 85, 90, 95, 97, 98, 99, or 100% identical to SEQ ID NO: 160, and the V_{L} sequence is at least 80, 85, 90, 95, 97, 98, 99, or 100% identical to SEQ ID NO: 161;
the V_{H} sequence is at least 80, 85, 90, 95, 97, 98, 99, or 100% identical to SEQ ID NO: 162, and the V_{L} sequence is at least 80, 85, 90, 95, 97, 98, 99, or 100% identical to SEQ ID NO: 163;
the V_{H} sequence is at least 80, 85, 90, 95, 97, 98, 99, or 100% identical to SEQ ID NO: 164, and the V_{L} sequence is at least 80, 85, 90, 95, 97, 98, 99, or 100% identical to SEQ ID NO: 165;
the V_{H} sequence is at least 80, 85, 90, 95, 97, 98, 99, or 100% identical to SEQ ID NO: 166, and the V_{L} sequence is at least 80, 85, 90, 95, 97, 98, 99, or 100% identical to SEQ ID NO: 167;
the V_{H} sequence is at least 80, 85, 90, 95, 97, 98, 99, or 100% identical to SEQ ID NO: 168, and the V_{L} sequence is at least 80, 85, 90, 95, 97, 98, 99, or 100% identical to SEQ ID NO: 169;
the V_{H} sequence is at least 80, 85, 90, 95, 97, 98, 99, or 100% identical to SEQ ID NO: 67, and the V_{L} sequence is at least 80, 85, 90, 95, 97, 98, 99, or 100% identical to SEQ ID NO: 68;
the V_{H} sequence is at least 80, 85, 90, 95, 97, 98, 99, or 100% identical to SEQ ID NO: 69, and the V_{L} sequence is at least 80, 85, 90, 95, 97, 98, 99, or 100% identical to SEQ ID NO: 70;
the V_{H} sequence is at least 80, 85, 90, 95, 97, 98, 99, or 100% identical to SEQ ID NO: 71, and the V_{L} sequence is at least 80, 85, 90, 95, 97, 98, 99, or 100% identical to SEQ ID NO: 72;
the V_{H} sequence is at least 80, 85, 90, 95, 97, 98, 99, or 100% identical to SEQ ID NO: 73, and the V_{L} sequence is at least 80, 85, 90, 95, 97, 98, 99, or 100% identical to SEQ ID NO: 74;
the V_{H} sequence is at least 80, 85, 90, 95, 97, 98, 99, or 100% identical to SEQ ID NO: 75, and the V_{L} sequence is at least 80, 85, 90, 95, 97, 98, 99, or 100% identical to SEQ ID NO: 76;
the V_{H} sequence is at least 80, 85, 90, 95, 97, 98, 99, or 100% identical to SEQ ID NO: 77, and the V_{L} sequence is at least 80, 85, 90, 95, 97, 98, 99, or 100% identical to SEQ ID NO: 78;
the V_{H} sequence is at least 80, 85, 90, 95, 97, 98, 99, or 100% identical to SEQ ID NO: 79, and the V_{L} sequence is at least 80, 85, 90, 95, 97, 98, 99, or 100% identical to SEQ ID NO: 80;
the V_{H} sequence is at least 80, 85, 90, 95, 97, 98, 99, or 100% identical to SEQ ID NO: 81, and the V_{L} sequence is at least 80, 85, 90, 95, 97, 98, 99, or 100% identical to SEQ ID NO: 82;
the V_{H} sequence is at least 80, 85, 90, 95, 97, 98, 99, or 100% identical to SEQ ID NO: 83, and the V_{L} sequence is at least 80, 85, 90, 95, 97, 98, 99, or 100% identical to SEQ ID NO: 84;
the V_{H} sequence is at least 80, 85, 90, 95, 97, 98, 99, or 100% identical to SEQ ID NO: 85, and the V_{L} sequence is at least 80, 85, 90, 95, 97, 98, 99, or 100% identical to SEQ ID NO: 86; or
the V_{H} sequence is at least 80, 85, 90, 95, 97, 98, 99, or 100% identical to SEQ ID NO: 87, and the V_{L} sequence is at least 80, 85, 90, 95, 97, 98, 99, or 100% identical to SEQ ID NO: 88.

In some embodiments, the V_{H} and V_{L} sequences of an antibody or antigen-binding fragment are as follows:
the V_{H} sequence comprises SEQ ID NO: 170, and the V_{L} sequence comprises SEQ ID NO: 171 (see Table E7 for definition of “X” residues);
the V_{H} sequence comprises SEQ ID NO: 160, and the V_{L} sequence comprises SEQ ID NO: 161;
the V_{H} sequence comprises SEQ ID NO: 162, and the V_{L} sequence comprises SEQ ID NO: 163;
the V_{H} sequence comprises SEQ ID NO: 164, and the V_{L} sequence comprises SEQ ID NO: 165;
the V_{H} sequence comprises SEQ ID NO: 166, and the V_{L} sequence comprises SEQ ID NO: 167;
the V_{H} sequence comprises SEQ ID NO: 168, and the V_{L} sequence comprises SEQ ID NO: 169;
the V_{H} sequence comprises SEQ ID NO: 67, and the V_{L} sequence comprises SEQ ID NO: 68;
the V_{H} sequence comprises SEQ ID NO: 69, and the V_{L} sequence comprises SEQ ID NO: 70;
the V_{H} sequence comprises SEQ ID NO: 71, and the V_{L} sequence comprises SEQ ID NO: 72;
the V_{H} sequence comprises SEQ ID NO: 73, and the V_{L} sequence comprises SEQ ID NO: 74;
the V_{H} sequence comprises SEQ ID NO: 75, and the V_{L} sequence comprises SEQ ID NO: 76;
the V_{H} sequence comprises SEQ ID NO: 77, and the V_{L} sequence comprises SEQ ID NO: 78;
the V_{H} sequence comprises SEQ ID NO: 79, and the V_{L} sequence comprises SEQ ID NO: 80;
the V_{H} sequence comprises SEQ ID NO: 81, and the V_{L} sequence comprises SEQ ID NO: 82;
the V_{H} sequence comprises SEQ ID NO: 83, and the V_{L} sequence comprises SEQ ID NO: 84;
the V_{H} sequence comprises SEQ ID NO: 85, and the V_{L} sequence comprises SEQ ID NO: 86; or
the V_{H} sequence comprises SEQ ID NO: 87, and the V_{L} sequence comprises SEQ ID NO: 88.

Also included are variants thereof, for example, variants having 1, 2, 3, 4, or 5 alterations in one or more framework regions, which bind to a human NRP2a polypeptide or epitope thereof (see, for example, Table N1, Table N2). Exemplary “alterations” include amino acid substitutions, additions, and deletions.

Merely for illustrative purposes, the binding interactions between an antibody or antigen-binding fragment thereof, a human NRP2a polypeptide (e.g., NRP2a v1 and/or v2), and/or an NRP2 ligand (e.g., CCL21, CCR7) can be detected and quantified using a variety of routine methods, including octet and Biacore assays (for example, with appropriately tagged soluble reagents, bound to a sensor chip), FACS analyses with cells expressing a NRP2a polypeptide on the cell surface (either native, or recombinant), immunoassays, fluorescence staining assays, ELISA assays, and microcalorimetry approaches such as ITC (isothermal Titration Calorimetry). See also the Examples.

In certain embodiments, an antibody or antigen-binding fragment thereof comprises variant or otherwise modified Fc region(s), including those having altered properties or biological activities relative to wild-type Fc region(s). Examples of modified Fc regions include those having mutated sequences, for instance, by substitution, insertion, deletion, or truncation of one or more amino acids relative to a wild-type sequence, hybrid Fc polypeptides composed of domains from different immunoglobulin classes/subclasses. Fc polypeptides having altered glycosylation/sialylation patterns, and Fc polypeptides that are modified or derivatized, for example, by biotinylation (see, e.g., U.S. Application No. 2010/0209424), phosphorylation, sulfation, etc., or any combination of the foregoing. Such modifications can be employed to alter (e.g., increase, decrease) the binding properties of the Fc region to one or more particular FcRs (e.g., FcγRI, FcγRIIa, FcγRIIb, FcγRIIc, FcγRIIIa, FcγRIIIb, FcRn), its pharmacokinetic properties (e.g., stability or half-life, bioavailability, tissue distribution, volume of distribution, concentration, elimination rate constant, elimination rate, area under the curve (AUC), clearance, Cₘₐₓ, tₘₐₓ, Cₘᵢₙ, fluctuation), its immunogenicity, its complement fixation or activation, and/or the CDC/ADCC/ADCP-related activities of the Fc region, among other properties described herein, relative to a corresponding wild-type Fc sequence of an antibody or antigen-binding fragment thereof. Included are modified Fc regions of human and/or mouse origin.

Also included are antibodies or antigen-binding fragments thereof that comprise hybrid Fc regions, for example, Fc regions that comprise a combination of Fc domains (e.g., hinge, CH₂, CH₃, CH₄) from immunoglobulins of different species (e.g., human, mouse), different Ig classes, and/or different Ig subclasses. General examples include hybrid Fc regions that comprise, consist of, or consist essentially of the following combination of CH₂/CH3 domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgE/IgA1, IgE/IgA2, IgE/IgD, IgE/IgE, IgE/IgG1, IgE/IgG2, IgE/IgG3, IgE/IgG4, IgE/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM, IgM/IgA1, IgM/IgA2, IgM/IgD, IgM/IgE, IgM/IgG1, IgM/IgG2, IgM/IgG3, IgM/IgG4, IgM/IgM (or fragments or variants thereof), and optionally include a hinge from one or more of IgA1, IgA2. IgD, IgG1, IgG2, IgG3, or IgG4, and/or a CH₄ domain from IgE and/or IgM. In specific embodiments, the hinge, CH₂. CH₃, and CH₄ domains are from human Ig.

Additional examples include hybrid Fc regions that comprise, consist of, or consist essentially of the following combination of CH₂/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₃ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Certain examples include hybrid Fc regions that comprise, consist of, or consist essentially of the following combination of CH₃/CH₄ domains: IgA1/IgE. IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₂ domain from one or more of IgA1, IgA2, IgD. IgE, IgG1, IgG2, IgG3, IgG4, or IgM. In specific embodiments, the hinge, CH₂, CH₃, and CH₄ domains are from human Ig.

Particular examples include hybrid Fc regions that comprise, consist of, or consist essentially of the following combination of hinge/CH₂ domains: IgA1/IgA1, IgA1/IgA2, IgA1/IgD, IgA1/IgE. IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM. IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₃ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Certain examples include hybrid Fc regions that comprise, consist of, or consist essentially of the following combination of hinge/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD. IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₂ domain from one or more of IgA1, IgA2, IgD, IgE, IgG1, IgG2, IgG3, IgG4, or IgM, and/or a CH₄ domain from IgE and/or IgM. In specific embodiments, the hinge. CH₂, CH₃, and CH₄ domains are from human Ig.

Some examples include hybrid Fc regions that comprise, consist of, or consist essentially of the following combination of hinge/CH₄ domains: IgA1/IgE, IgA1/IgM, IgA2/IgE, IgA2/IgM, IgD/IgE, IgD/IgM, IgG1/IgE, IgG1/IgM, IgG2/IgE, IgG2/IgM, IgG3/IgE, IgG3/IgM, IgG4/IgE, IgG4/IgM (or fragments or variants thereof), and optionally include a CH₂ domain from one or more of IgA1, IgA2, IgD, IgE, IgG1, IgG2, IgG3, IgG4, or IgM, and/or a CH₃ domain from one or more of IgA1, IgA2, IgD, IgE, IgG1, IgG2, IgG3, IgG4, or IgM.

Specific examples of hybrid Fc regions can be found, for example, in WO 2008/147143, which are derived from combinations of IgG subclasses or combinations of human IgD and IgG.

Also included are antibodies or antigen-binding fragments thereof having derivatized or otherwise modified Fc regions. In certain aspects, the Fc region may be modified by phosphorylation, sulfation, acrylation, glycosylation, methylation, farnesylation, acetylation, amidation, and the like, for instance, relative to a wild-type or naturally-occurring Fc region. In certain embodiments, the Fc region may comprise wild-type or native glycosylation patterns, or alternatively, it may comprise increased glycosylation relative to a native form, decreased glycosylation relative to a native form, or it may be entirely deglycosylated. As one example of a modified Fc glycoform, decreased glycosylation of an Fc region reduces binding to the C1q region of the first complement component C1, a decrease in ADCC-related activity, and/or a decrease in CDC-related activity. Certain embodiments thus employ a deglycosylated or aglycosylated Fc region. See, e.g., WO 2005/047337 for the production of exemplary aglycosylated Fc regions. Another example of an Fc region glycoform can be generated by substituting the Q295 position with a cysteine residue (see, e.g., U.S. Application No. 2010/0080794), according to the Kabat et al. numbering system. Certain embodiments may include Fc regions where about 80-100% of the glycoprotein in Fc region comprises a mature core carbohydrate structure that lacks fructose (see, e.g., U.S. Application No. 2010/0255013). Some embodiments include Fc regions that are optimized by substitution or deletion to reduce the level of fucosylation, for instance, to increase affinity for FcγRI. FcγRIa, or FcγRIIIa, and/or to improve phagocytosis by FcγRIIa-expressing cells (see U.S. Application Nos. 2010/0249382 and 2007/0148170).

As another example of a modified Fc glycoform, an Fc region of an antibody or antigen-binding fragment thereof may comprise oligomannose-type N-glycans, and optionally have one or more of the following: increased ADCC effector activity, increased binding affinity for FcγRIIIA (and certain other FcRs), similar or increased binding specificity for the target of the NRP2a polypeptide, similar or higher binding affinity for the target of the NRP2a polypeptide, and/or similar or lower binding affinity for mannose receptor, relative to a corresponding Fc region that contains complex-type N-glycans (see, e.g., U.S. Application No. 2007/0092521 and U.S. Pat. No. 7,700,321). As another example, enhanced affinity of Fc regions for FcγRs has been achieved using engineered glycoforms generated by expression of antibodies in engineered or variant cell lines (see, e.g., Umana et al., Nat Biotechnol. 17:176-180, 1999; Davies et al., Biotechnol Bioeng. 74:288-294, 2001; Shields et al., J Biol Chem. 277:26733-26740, 2002; Shinkawa et al., 2003. J Biol Chem. 278:3466-3473, 2003; and U.S. Application No. 2007/0111281). Certain Fc region glycoforms comprise an increased proportion of N-glycoside bond type complex sugar chains, which do not have the 1-position of fucose bound to the 6-position of N-acetylglucosamine at the reducing end of the sugar chain (see, e.g., U.S. Application No. 2010/0092997). Particular embodiments may include IgG Fc region that is glycosylated with at least one galactose moiety connected to a respective terminal sialic acid moiety by an α-2,6 linkage, optionally where the Fc region has a higher anti-inflammatory activity relative to a corresponding, wild-type Fc region (see U.S. Application No. 2008/0206246). Certain of these and related altered glycosylation approaches have generated substantial enhancements of the capacity of Fc regions to selectively bind FcRs such as FcγRIII, to mediate ADCC, and to alter other properties of Fc regions, as described herein.

Certain variant, fragment, hybrid, or otherwise modified Fc regions of an antibody or antigen-binding fragment thereof may have altered binding to one or more FcRs, and/or corresponding changes to effector function, relative to a corresponding, wild-type Fc sequence (e.g., same species, same Ig class, same Ig subclass). For instance, such Fc regions may have increased binding to one or more of Fcγ receptors, Fcα receptors, Fcε receptors, and/or the neonatal Fc receptor, relative to a corresponding, wild-type Fc sequence. In other embodiments, variant, fragment, hybrid, or modified Fc regions may have decreased binding to one or more of Fcγ receptors, Fcα receptors, Fcε receptors, and/or the neonatal Fc receptor, relative to a corresponding, wild-type Fc sequence. Specific FcRs are described elsewhere herein.

In some embodiments, an antibody comprises an Fc domain, comprising one or more mutations to increase binding to one or more of Fcγ receptors. Fcα receptors. Fcε receptors, and/or the neonatal Fc receptor, relative to a corresponding, wild-type Fc sequence. In some embodiments, an antibody comprises an IgG1 or IgG3 Fc domain, comprising one or more mutations to increase binding to one or more of Fcγ receptors, Fcα receptors, Fcε receptors, and/or the neonatal Fc receptor, relative to a corresponding, wild-type Fc sequence. In some embodiments, an antibody comprises an Fc domain, comprising one or more mutations to increase effector function. In some embodiments the at least one antibody comprises an Fc domain selected from a human IgG1 and IgG3, comprising one or more mutations to increase effector function.

In some embodiments, an antibody is blocking antibody that comprises an Fc domain with high effector activity. In some embodiments, the blocking antibody comprises an Fc domain selected from a human IgG1 and IgG3, comprising one or more mutations to increase effector function. In some embodiments, an antibody is a partial-blocking antibody that comprises an Fc domain with high effector activity. In some embodiments, the a partial-blocking antibody comprises an Fc domain selected from a human IgG1 and IgG3, comprising one or more mutations to increase effector function. In some embodiments, an antibody is a non-blocking antibody that comprises an Fc domain with high effector activity. In some embodiments, the non-blocking antibody comprises an Fc domain selected from a human IgG1 or IgG3, comprising one or more mutations to increase effector function.

In some embodiments, an antibody comprises an Fc domain, comprising one or more mutations to decrease binding to one or more of Fcγ receptors, Fcα receptors. Fcε receptors, and/or the neonatal Fc receptor, relative to a corresponding, wild-type Fc sequence. In some embodiments, an antibody comprises an IgG1 or IgG3 Fc domain, comprising one or more mutations to decrease binding to one or more of Fcγ receptors, Fcα receptors, Fcε receptors, and/or the neonatal Fc receptor, relative to a corresponding, wild-type Fc sequence. In some embodiments, an antibody comprises an Fc domain, comprising one or more mutations to decrease effector function. In some embodiments, an antibody comprises an Fc domain selected from a human IgG2 and IgG4, comprising one or more mutations to decrease effector function.

In some embodiments, an antibody is a blocking antibody comprising an Fc domain with low effector activity. In some embodiments, the blocking antibody comprises an Fc domain selected from a human IgG2 and IgG4, comprising one or more mutations to decrease effector function. In some embodiments, an antibody is a partial-blocking antibody comprising an Fc domain with low effector activity. In some embodiments, the partial-blocking antibody comprises an Fc domain selected from a human IgG2 and IgG4, comprising one or more mutations to decrease effector function. In some embodiments, an antibody is a non-blocking antibody comprising an Fc domain with low effector activity. In some embodiments, the non-blocking antibody comprises an Fc domain selected from a human IgG2 and IgG4, comprising one or more mutations to decrease effector function.

Specific examples of Fc variants having altered (e.g., increased, decreased) effector function/FcR binding can be found, for example, in U.S. Pat. Nos. 5,624,821 and 7,425,619; U.S. Application Nos. 2009/0017023, 2009/0010921, and 2010/0203046; and WO 2000/42072 and WO 2004/016750. Certain examples include human Fc regions having a one or more substitutions at position 298, 333, and/or 334, for example, S298A, E333A, and/or K334A (based on the numbering of the EU index of Kabat et al.), which have been shown to increase binding to the activating receptor FcγRIIIa and reduce binding to the inhibitory receptor FcγRIIb. These mutations can be combined to obtain double and triple mutation variants that have further improvements in binding to FcRs. Certain embodiments include a S298A/E333A/K334A triple mutant, which has increased binding to FcγRIIIa, decreased binding to FcγRIIb, and increased ADCC (see, e.g., Shields et al., J Biol Chem. 276:6591-6604, 2001; and Presta et al., Biochem Soc Trans. 30:487-490, 2002). See also engineered Fc glycoforms that have increased binding to FcRs, as disclosed in Umana et al., supra; and U.S. Pat. No. 7,662,925. Some embodiments include Fc regions that comprise one or more substitutions selected from 434S, 252Y/428L, 252Y/434S, and 428L/434S (see U.S. Application Nos. 2009/0163699 and 20060173170), based on the EU index of Kabat et al.

Certain variant, fragment, hybrid, or modified Fc regions may have altered effector functions, relative to a corresponding, wild-type Fc sequence. For example, such Fc regions may have increased complement fixation or activation, increased C1q binding affinity, increased CDC-related activity, increased ADCC-related activity, and/or increased ADCP-related activity, relative to a corresponding, wild-type Fc sequence. In other embodiments, such Fc regions may have decreased complement fixation or activation, decreased C1q binding affinity, decreased CDC-related activity, decreased ADCC-related activity, and/or decreased ADCP-related activity, relative to a corresponding, wild-type Fc sequence. As merely one illustrative example, an Fc region may comprise a deletion or substitution in a complement-binding site, such as a C1q-binding site, and/or a deletion or substitution in an ADCC site. Examples of such deletions/substitutions are described, for example, in U.S. Pat. No. 7,030,226. Many Fc effector functions, such as ADCC, can be assayed according to routine techniques in the art. (see, e.g., Zuckerman et al., CRC Crit Rev Microbiol. 7:1-26, 1978). Useful effector cells for such assays includes, but are not limited to, natural killer (NK) cells, macrophages, and other peripheral blood mononuclear cells (PBMC). Alternatively, or additionally, certain Fc effector functions may be assessed in vivo, for example, by employing an animal model described in Clynes et al. PNAS. 95:652-656, 1998.

Certain variant hybrid, or modified Fc regions may have altered stability or half-life relative to a corresponding, wild-type Fc sequence. In certain embodiments, such Fc regions may have increased half-life relative to a corresponding, wild-type Fc sequence. In other embodiments, variant hybrid, or modified Fc regions may have decreased half-life relative to a corresponding, wild-type Fc sequence. Half-life can be measured in vitro (e.g., under physiological conditions) or in vivo, according to routine techniques in the art, such as radiolabeling, ELISA, or other methods. In vivo measurements of stability or half-life can be measured in one or more bodily fluids, including blood, serum, plasma, urine, or cerebrospinal fluid, or a given tissue, such as the liver, kidneys, muscle, central nervous system tissues, bone, etc. As one example, modifications to an Fc region that alter its ability to bind the FcRn can alter its half-life in vivo. Assays for measuring the in vivo pharmacokinetic properties (e.g., in vivo mean elimination half-life) and non-limiting examples of Fc modifications that alter its binding to the FcRn are described, for example, in U.S. Pat. Nos. 7,217,797 and 7,732,570; and U.S. Application Nos. US 2010/0143254 and 2010/0143254.

Additional non-limiting examples of modifications to alter stability or half-life include substitutions/deletions at one or more of amino acid residues selected from 251-256, 285-290, and 308-314 in the CH₂ domain, and 385-389 and 428-436 in the CH₃ domain, according to the numbering system of Kabat et al. See U.S. Application No. 2003/0190311. Specific examples include substitution with leucine at position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, and/or substitution with histidine, tyrosine, arginine or threonine at position 436, including any combination thereof. Such modifications optionally increase affinity of the Fc region for the FcRn and thereby increase half-life, relative to a corresponding, wild-type Fc region.

Certain variant hybrid, or modified Fc regions may have altered solubility relative to a corresponding, wild-type Fc sequence. In certain embodiments, such Fc regions may have increased solubility relative to a corresponding, wild-type Fc sequence. In other embodiments, variant hybrid, or modified Fc regions may have decreased solubility relative to a corresponding, wild-type Fc sequence. Solubility can be measured, for example, in vitro (e.g., under physiological conditions) according to routine techniques in the art. Exemplary solubility measurements are described elsewhere herein.

Additional examples of variants include IgG Fc regions having conservative or non-conservative substitutions (as described elsewhere herein) at one or more of positions 250, 314, or 428 of the heavy chain, or in any combination thereof, such as at positions 250 and 428, or at positions 250 and 314, or at positions 314 and 428, or at positions 250, 314, and 428 (see, e.g., U.S. Application No. 2011/0183412). In specific embodiments, the residue at position 250 is substituted with glutamic acid or glutamine, and/or the residue at position 428 is substituted with leucine or phenylalanine. As another illustrative example of an IgG Fc variant, any one or more of the amino acid residues at positions 214 to 238, 297 to 299, 318 to 322, and/or 327 to 331 may be used as a suitable target for modification (e.g., conservative or non-conservative substitution, deletion). In particular embodiments, the IgG Fc variant CH₂ domain contains amino acid substitutions at positions 228, 234, 235, and/or 331 (see, e.g., human IgG4 with Ser228Pro and Leu235Ala mutations) to attenuate the effector functions of the Fc region (see, e.g., U.S. Pat. No. 7,030,226). Here, the numbering of the residues in the heavy chain is that of the EU index (see Kabat et al., “Sequences of Proteins of Immunological Interest,” 5′ Ed., National Institutes of Health, Bethesda, Md. (1991)). Certain of these and related embodiments have altered (e.g., increased, decreased) FcRn binding and/or serum half-life, optionally without reduced effector functions such as ADCC or CDC-related activities.

Additional examples include variant Fc regions that comprise one or more amino acid substitutions at positions 279, 341, 343 or 373 of a wild-type Fc region, or any combination thereof (see, e.g., U.S. Application No. 2007/0224188). The wild-type amino acid residues at these positions for human IgG are valine (279), glycine (341), proline (343) and tyrosine (373). The substation(s) can be conservative or non-conservative, or can include non-naturally occurring amino acids or mimetics, as described herein. Alone or in combination with these substitutions, certain embodiments may also employ a variant Fc region that comprises at least 1, 2, 3, 4, 5, 6, 7, 8, 9, 10 or more amino acid substitutions selected from the following: 235G, 235R, 236F, 236R, 236Y, 237K, 237N, 237R, 238E, 238G, 238H, 238I, 238L, 238V, 238W, 238Y, 244L, 245R, 247A, 247D, 247E, 247F, 247M, 247N, 247Q, 247R, 247S, 247T, 247W, 247Y, 248F, 248P, 248Q, 248W, 249L, 249M, 249N, 249P, 249Y, 251H, 251I, 251W, 254D, 254E, 254F, 254G, 254H, 254I, 254K, 254L, 254M, 254N, 254P, 254Q, 254R, 254V, 254W, 254Y, 255K, 255N, 256H, 256I, 256K, 256L, 256V, 256W, 256Y, 257A, 257I, 257M, 257N, 257S, 258D, 260S, 262L, 264S, 265K, 265S, 267H, 267I, 267K, 268K, 269N, 269Q, 271T, 272H, 272K, 272L, 272R, 279A, 279D, 279F, 279G, 279H, 279I, 279K, 279L, 279M, 279N, 279Q, 279R, 279S, 279T, 279W, 279Y, 280T, 283F, 283G, 283H, 283I, 283K, 283L, 283M, 283P, 283R, 283T, 283W, 283Y, 285N, 286F, 288N, 288P, 292E, 292F, 292G, 292I, 292L, 293S, 293V, 301W, 304E, 307E, 307M, 312P, 315F, 315K, 315L, 315P, 315R, 316F, 316K, 317P, 317T, 318N, 318P, 318T, 332F, 332G, 332L, 332M, 332S, 332V, 332W, 339D, 339E, 339F, 339G, 339H, 339I, 339K, 339L, 339M, 339N, 339Q, 339R, 339S, 339W, 339Y, 341D, 341E, 341F, 341H, 341I, 341K, 341L, 341M, 341N, 341P, 341Q, 341R, 341S, 341T, 341V, 341W, 341Y. 343A, 343D, 343E, 343F, 343G, 343H, 343I, 343K, 343L, 343M, 343N, 343Q, 343R, 343S, 343T, 343V, 343W, 343Y, 373D, 373E, 373F, 373G, 373H, 373I, 373K, 373L, 373M, 373N, 373Q, 373K, 373S, 373T, 373V, 373W, 375R, 376E, 376F, 376G, 376H, 376I, 376L, 376M, 376N, 376P, 376Q, 376R, 376S, 376T, 376V, 376W, 376Y, 377G, 377K, 377P, 378N, 379N, 379Q, 379S, 379T, 380D, 380N, 380S, 380T, 382D, 382F, 382H, 382I, 382K, 382L, 382M, 382N, 382P, 382Q, 382R, 382S, 382T, 382V, 382W, 382Y, 385E, 385P, 386K, 423N, 424H, 424M, 424V, 426D, 426L, 427N, 429A, 429F, 429M, 430A, 430D, 430F, 430G, 430H, 430I, 430K, 430L, 430M, 430N, 430P, 430Q, 430R, 430S, 430T, 430V, 430W, 430Y, 431H, 431K, 431P, 432R, 432S, 438G, 438K, 438L, 438T, 438W, 439E, 439H, 439Q, 440D, 440E, 440F, 440G, 440H, 440I, 440K, 440L, 440M, 440Q, 440T, 440V or 442K. As above, the numbering of the residues in the heavy chain is that of the EU index (see Kabat et al., supra). Such variant Fc regions typically confer an altered effector function or altered serum half-life upon the antibody to which the variant Fc region is operably attached. Preferably the altered effector function is an increase in ADCC, a decrease in ADCC, an increase in CDC, a decrease in CDC, an increase in C1q binding affinity, a decrease in C1q binding affinity, an increase in FcR (preferably FcRn) binding affinity or a decrease in FcR (preferably FcRn) binding affinity as compared to a corresponding Fc region that lacks such amino acid substitution(s).

Additional examples include variant Fc regions that comprise an amino acid substitution at one or more of position(s) 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 293, 294, 295, 296, 297, 298, 299, 300, 302, 313, 317, 318, 320, 322, 323, 324, 325, 326, 327, 328, 329, 330, 331, 332, 333, 334, 335 336 and/or 428 (see, e.g., U.S. Pat. No. 7,662,925). In specific embodiments, the variant Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K3261, K326T, L328M, L328I, L328Q, L328D, L328V, L328T, A330Y, A330L, A330I, 1332D, I332E, 1332N, 1332Q, T335D, T335R, and T335Y. In other specific embodiments, the variant Fc region comprises at least one amino acid substitution selected from the group consisting of: V264I, F243L/V264I, L328M, I332E, L328M/I332E, V264I/I332E, S298A/I332E, S239E/I332E, S239Q/I332E, S239E. A330Y, 1332D, L328I/I332E, L328Q/I332E, V264T, V240I, V266I, S239D, S239D/1332D, S239D/I332E, S239D, I332N, S239D/1332Q, S239E/1332D, S239E/1332N, S239E/1332Q, S239N/1332D, S239N/I332E, S239Q/1332D, A330Y/I332E, V264L, A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K3261, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, In more specific embodiments, the variant Fc region comprises a series of substitutions selected from the group consisting of: N297D/I332E, F241Y/F243Y/V262T/V264T/N297D/I332E, S239D/N297D/I332E, S239E/N297D/I332E, S239D/D265Y/N297D/I332E, S239D/D265H/N297D/I332E, V264E/N297D/I332E, Y296N/N297D/I332E, N297D/A330Y/I332E, S239D/D265V/N297D/I332E, S239D/D2651/N297D/I332E, and N297D/S298A/A330Y/I332E. In specific embodiments, the variant Fc region comprises an amino acid substitution at position 332 (using the numbering of the EU index, Kabat et al., supra). Examples of substitutions include 332A, 332D, 332E, 332F, 332G, 332H, 332K, 332L, 332M, 332N, 332P, 332Q, 332R, 332S, 332T, 332V, 332W and 332Y. The numbering of the residues in the Fc region is that of the EU index of Kabat et al. Among other properties described herein, such variant Fc regions may have increased affinity for an FcγR, increased stability, and/or increased solubility, relative to a corresponding, wild-type Fc region.

Further examples include variant Fc regions that comprise one or more of the following amino acid substitutions: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 3351, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, optionally where the variant has altered recognition of an Fc ligand and/or altered effector function compared with a parent Fc polypeptide, and wherein the numbering of the residues is that of the EU index as in Kabat et al. Specific examples of these and related embodiments include variant Fc regions that comprise or consist of the following sets of substitutions: (1) N276D, R292Q, V305A, I377V, T394A, V412A and K439E; (2) P244L, K246E, D399G and K409R; (3) S304G, K320R, S324T, K326E and M358T; (4) F243S, P247L, D265V, V266A, S383N and T411I; (5) H224N, F243L, T393A and H433P; (6) V240A, S267G, G341E and E356G; (7) M252T, P291L, P352A, R355W, N390D, S408G, S426F and A431S; (8) P228L, T289A, L365Q, N389S and 5440G; (9) F241L, V273A, K340Q and L441F; (10) F241L, T299A, 1332T and M428T; (11) E269K, Y300H, Q342R, V422I and G446A; (12) T225A, R301c, S304G, D312N, N315D, L351S and N421S; (13) S254T, L306I, K326R and Q362L; (14) H224Y, P230S, V323A, E333D, K338R and S364C; (15) T3351, K414M and P445R; (16) T3351 and K414M; (17) P247A, E258K, D280N, K288R, N297D, T299A, K322E, Q342R, S354A and L365P; (18) H268N, V279M, A339T, N361D and S426P; (19) C261Y, K290E, L306F, Q311R, E333G and Q438L; (20) E283G, N315K, E333G, R344Q, L365P and S442T; (21) Q347R, N361Y and K439R; (22) S239P, S254P, S267N, H285R, N315S, F372L, A378T, N390D, Y391C, F404S, E430K, L432P and K447E; and (23) E269G, Y278H, N325S and K370R, wherein the numbering of the residues is that of the EU index as in Kabat et al. (see, e.g., U.S. Application No. 2010/0184959).

Variant Fc regions can also have one or more mutated hinge regions, as described, for example, in U.S. Application No. 2003/0118592. For instance, one or more cysteines in a hinge region can be deleted or substituted with a different amino acid. The mutated hinge region can comprise no cysteine residues, or it can comprise 1, 2, or 3 fewer cysteine residues than a corresponding, wild-type hinge region. In some embodiments, an Fc region having a mutated hinge region of this type exhibits a reduced ability to dimerize, relative to a wild-type Ig hinge region.

In particular embodiments, the Fc region comprises, consists, or consists essentially of the Fc from human IgG4 or IgG4 (see, e.g., Allberse and Schuurman, Immunology. 105:9-19, 2002), or a fragment or variant thereof. Table F1 below provides exemplar), sequences (CH1, hinge (underlined), CH2, and CH3 regions) from human IgG1 and IgG4. Examples of variant IgG4 sequences that can be employed are described, for example, in Peters et al., JBC. 287:24525-24533, 2012, and include substitutions at C227, C230, C127 (e.g., C127S), and C131 (e.g., C131S). Other variants that can be used include a L445P substitution in IgG4 (denoted as IgG4-2) or a D356E and L358M substitution in IgG1, (denoted as IgG1m(zf)).

TABLE Fl
Exemplary IgG4 Fc Sequences

| | | SEQ ID |
|---|---|---|
| Name | Sequence | NO: |
| Wild-type | ASTKGPSVFPLAPCSRSTSESTAALGCLVKDYFPEPVTVSWNSGALTSGVH | 109 |
| IgG4 | TEPAVLQSSGLYSLSSVVTVPSSSLGTKTYTCNVDHKPSNTKVDKRVESKY | |
| | GPPCPSCPAPEFLGGPSVFLFPPKPKDTIMISRTPEVTCVVVDVSQEDPEV | |
| | QFNWYVDGVEVHNAKTKPREEQFNSTYRVVSVLTVLHQDWINGKEYKCKVS | |
| | NKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVESCS | |
| | VMHEALHNHYTQKSLSLSLGK | |
| S241P | ASTKGPSVFPLAPCSRSTSESTAALGCLVKDYFPEPVTVSWNSGALTSGVH | 110 |
| | TFPAVLQSSGLYSLSSVVTVPSSSLGTKTYTCNVDHKPSNTKVDKRVESKY | |
| | GPPCPPCPAPEFLGGPSVFLFPPKPKDTLMISRTPEVTCVVVDVSQEDPEV | |
| | QFNWYVDGVEVHNAKTKPREEQFNSTYRVVSVLTVLHQDWLNGKEYKCKVS | |
| | NKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVESCS | |
| | VMHEALHNHYTQKSLSLSLGK | |
| IgG1m (za) | ASTKGPSVFPLAPSSKSTSGGTAALGCLVKDYFPEPVTVSWNSGALTSGVH | 111 |
| GenBank: | TFPAVLQSSGLYSLSSVVTVPSSSLGTQTYICNVNHKPSNTKVDKKVEPKS | |
| AH007035.2 | CDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALPAPIEKTISKAKGQPREPQVYTLPPSRDELTKNQVSLTCLVKGF | |
| | YPSDIAVEWESNGQPENNYKTTPPVLDSDGSFFLYSKLTVDKSRWQQGNVF | |
| | SCSVMHEALHNHYTQKSLSLSPGK | |
| Kappa | RTVAAPSVFIFPPSDEQLKSGTASVVCLLNNFYPREAKVQWKVDNALQSGN | 112 |
| Km3 | SQESVTEQDSKDSTYSLSSTLTLSKADYEKHKVYACEVTHQGLSSPVTKSF | |
| | NRGEC | |

As noted above, antibodies having altered Fc regions typically have altered (e.g., improved, increased, decreased) pharmacokinetic properties relative to corresponding wild-type Fc region. Examples of pharmacokinetic properties include stability or half-life, bioavailability (the fraction of a drug that is absorbed), tissue distribution, volume of distribution (apparent volume in which a drug is distributed immediately after it has been injected intravenously and equilibrated between plasma and the surrounding tissues), concentration (initial or steady-state concentration of drug in plasma), elimination rate constant (rate at which drugs are removed from the body), elimination rate (rate of infusion required to balance elimination), area under the curve (AUC or exposure; integral of the concentration-time curve, after a single dose or in steady state), clearance (volume of plasma cleared of the drug per unit time), Cₘₐₓ (peak plasma concentration of a drug after oral administration), tₘₐₓ (time to reach Cₘₐₓ), Cₘᵢₙ (lowest concentration that a drug reaches before the next dose is administered), and fluctuation (peak trough fluctuation within one dosing interval at steady state).

In particular embodiments, an antibody or antigen-binding fragment thereof has a biological half life at about pH 7.4, at about a physiological pH, at about 25° C., or room temperature, and/or at about 37° C., or human body temperature (e.g., in vivo, in serum, in a given tissue, in a given species such as rat, mouse, monkey, or human), of about or at least about 30 minutes, about 1 hour, about 2 hour, about 3 hours, about 4 hours, about 5 hours, about 6 hours, about 12 hours, about 18 hours, about 20 hours, about 24 hours, about 30 hours, about 36 hours, about 40 hours, about 48 hours, about 50 hours, about 60 hours, about 70 hours, about 72 hours, about 80 hours, about 84 hours, about 90 hours, about 96 hours, about 120 hours, or about 144 hours or more, or about 1 week, or about 2 weeks, or about 3 weeks, or about 4 weeks, or about 5 weeks, or about 6 weeks or more, or any intervening half-life, including all ranges in between.

In some embodiments, an antibody or antigen-binding fragment thereof has a T, of about or at least about 60, 62, 64, 66, 68, 70, 72, 74, or 75° C. In some embodiments, an antibody or antigen-binding fragment thereof has a Tₘ of about 60° C., or greater.

In some embodiments, an antibody or antigen-binding fragment thereof conjugated to one or more cytotoxic or chemotherapeutic agents. General examples of cytotoxic or chemotherapeutic agents include, without limitation, alkylating agents, anti-metabolites, anthracyclines, anti-tumor antibiotics, platinums, type I topoisomerase inhibitors, type II topoisomerase inhibitors, vinca alkaloids, and taxanes. Specific examples of cytotoxic or chemotherapeutic agents include, without limitation, cyclophosphamide, cilengitide, lomustine (CCNU), melphalan, procarbazine, carmustine (BCNU), enzastaurin, busulfan, daunorubicin, doxorubicin, gefitinib, erlotinib idarubicin, temozolomide, epirubicin, mitoxantrone, bleomycin, cisplatin, carboplatin, oxaliplatin, camptothecins, irinotecan, topotecan, amsacrine, etoposide, etoposide phosphate, teniposide, temsirolimus, everolimus, vincristine, vinblastine, vinorelbine, vindesine, CT52923, paclitaxel, imatinib, dasatinib, sorafenib, pazopanib, sunitinib, vatalanib, gefitinib, erlotinib, AEE-788, dichloroacetate, tamoxifen, fasudil, SB-681323, semaxanib, donepazil, galantamine, memantine, rivastigmine, tacrine, rasagiline, naltrexone, lubiprostone, safmamide, istradefylline, pimavanserin, pitolisant, isradipine, pridopidine (ACR16), tetrabenazine, bexarotene, glatiramer acetate, fingolimod, and mitoxantrone, including pharmaceutically acceptable salts and acids thereof. Further examples of cytotoxic or chemotherapeutic agents include alkylating agents such as thiotepa, cyclophosphamide (CYTOXAN™); alkyl sulfonates such as busulfan, improsulfan and piposulfan; aziridines such as benzodopa, carboquone, meturedopa, and uredepa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton. N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above.

The antibodies or antigen-binding fragments thereof can be used in any of the compositions, methods, and/or kits described herein, and combined with one or more of the immunotherapy agents described herein.

### Additional Therapeutic Agents and Compositions

In certain embodiments, an antibody or antigen-binding fragment thereof is used in combination with one or more additional therapeutic agents, including immunotherapy agents, chemotherapeutic agents, hormonal therapeutic agents, and kinase inhibitors.

Immunotherapy Agents. Certain embodiments employ one or more cancer immunotherapy agents. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and a cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In particular embodiments, the immune checkpoint modulatory agent is a polypeptide or peptide. The terms “peptide” and “polypeptide” are used interchangeably herein, however, in certain instances, the term “peptide” can refer to shorter polypeptides, for example, polypeptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Polypeptides and peptides can be composed of naturally-occurring amino acids and/or non-naturally occurring amino acids, as described herein

Antibodies are also included as polypeptides. Thus, in some embodiments, the immune checkpoint modulatory polypeptide agent is an antibody or “antigen-binding fragment thereof”, as described elsewhere herein.

In some embodiments, the agent is or comprises a “ligand,” for example, a natural ligand, of the immune checkpoint molecule. A “ligand” refers generally to a substance or molecule that forms a complex with a target molecule (e.g., biomolecule) to serve a biological purpose, and includes a “protein ligand,” which generally produces a signal by binding to a site on a target molecule or target protein. Thus, certain agents are protein ligands that, in nature, bind to an immune checkpoint molecule and produce a signal. Also included are “modified ligands,” for example, protein ligands that are fused to a pharmacokinetic modifier, for example, an Fc region derived from an immunoglobulin.

The binding properties of polypeptides can be quantified using methods well known in the art (see Davies et al., Annual Rev. Biochem. 59:439-473, 1990). In some embodiments, a polypeptide specifically binds to a target molecule, for example, an immune checkpoint molecule or an epitope thereof, with an equilibrium dissociation constant that is about or ranges from about ≤10−7 to about 10−8 M. In some embodiments, the equilibrium dissociation constant is about or ranges from about ≤10−9 M to about ≤10−10 M. In certain illustrative embodiments, the polypeptide has an affinity (Kd or EC₅₀) for a target described herein (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the agent is a “small molecule,” which refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of about or less than about 1000-2000 Daltons, typically between about 300 and 700 Daltons, and including about or less than about 50, 100, 150, 200, 250, 300, 350, 400, 450, 500, 550, 500, 650, 600, 750, 700, 850, 800, 950, 1000 or 2000 Daltons.

Certain small molecules can have the “specific binding” characteristics described for herein polypeptides such as antibodies. For instance, in some embodiments a small molecule specifically binds to a target, for example, an immune checkpoint molecule, with a binding affinity (Kd or EC₅₀) of about, at least about, or less than about. 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 201210039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see. e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun. 33: 970-9, 2003; and Workman et al., Journal of Immun. 172: 5450-5, 2004), and has been reported to play a role in Treg suppressive function (see. e.g., Huang et al., Immunity. 21: 503-13, 20(4). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a VISTA antagonist or inhibitor. V-domain Ig suppressor of T cell activation (VISTA) is primarily expressed on hematopoietic cells and is an inhibitory immune checkpoint regulator that suppresses T-cell activation, induces Foxp3 expression, and is highly expressed within the tumor microenvironment where it suppresses anti-tumor T cell responses (see, e.g., Lines et al., Cancer Res. 74:1924-32, 2014). General examples of VISTA antagonists or inhibitors include an antibody or antigen-binding fragment or small molecule that specifically binds to VISTA and reduces one or more of its immune-suppressive activities.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derré et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD1600 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz. J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014:3; e27255. doi:10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the cancer immunotherapy agent is a cancer vaccine. Exemplary cancer vaccines include Oncophage, human papillomavirus HPV vaccines such Gardasil or Cervarix, hepatitis B vaccines such as Engerix-B, Recombivax HB, or Twinrix, and sipuleucel-T (Provenge). In some embodiments, the cancer vaccine comprises or utilizes one or more cancer antigens, or cancer-associate d antigens. Exemplary cancer antigens include, without limitation, human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, VEGFR-3, NRP2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen. TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In certain embodiments, the cancer immunotherapy agent is an oncolytic virus. An oncolytic virus is a virus that preferentially infects and kills cancer cells. Included are naturally-occurring and man-made or engineered oncolytic viruses. Most oncolytic viruses are engineered for tumor selectivity, although there are naturally-occurring examples such as Reovirus and the SVV-001 Seneca Valley virus. General examples of oncolytic viruses include VSV, Poliovirus, Reovirus, Senecavirus, and RIGVIR, and engineered versions thereof. Non-limiting examples of oncolytic viruses include herpes simplex virus (HSV) and engineered version thereof, talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-240I, among others.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a T-cell based adoptive immunotherapy. In some embodiments, the cell-based immunotherapy comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015).

In certain instances, the cancer to be treated associates with the cancer antigen, that is, the cancer antigen-specific T-cells are targeted against or enriched for at least one antigen that is known to associate with the cancer to be treated. In some embodiments, the cancer antigen is selected from one or more of CD19, human Her2/neu, Her1/EGF receptor (EGFR), Her3, A33 antigen, B7H3, CD5, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C. NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

Additional exemplary cancer antigens include 5T4, 707-AP, 9D7, AFP, AIbZIP HPG1, alpha-5-beta-1-integrin, alpha-5-beta-6-integrin, alpha-actinin4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R1 7I, HLA-A1 1/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6. MAGE-B10, MAGE-B1 6, MAGE-B1 7, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MCI R, M-CSF, ME1/m, mesothelin, MG50/PXDN, MMP1 1, MN/CA IX-antigen, MRP-3, MUC-1, MUC-2. MUM-1/m, MUM-2/m, MUM-3/m, myosin class 1/m, NA88-A, N-acetylglucosaminyltransferase-V, Neo-PAP, Neo-PAP/m, NFYC/m, NGEP, NMP22, NPM/ALK, N-Ras/m, NSE, NY-ESO-B, NY-ESO-1, OA1, OFA-iLRP, OGT, OGT/m, OS-9. OS-9/m, osteocalcin, osteopontin, pi 5, p190 minor bcr-abl, p53, p53/m, PAGE-4, PA1-1, PA1-2, PAP, PART-1, PATE, PDEF, Pim-1 Kinase, Pin-1, Pm1/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGF-beta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, and WT1. Certain preferred antigens include p53, CA 125, EGFR, Her2/neu, hTERT, PAP, MAGE-A1, MAGE-A3, Mesothelin, MUC-1, GPI00, MART-1, Tyrosinase, PSA, PSCA, PSMA, STEAP-1, Ras, CEA and WT1, and more preferably PAP, MAGE-A3, WT1, and MUC-1.

In some embodiments the antigen is selected from MAGE-A1 (e.g., MAGE-A1 according to accession number M77481), MAGE-A2, MAGE-A3. MAGE-A6 (e.g., MAGE-A6 according to accession number NM_005363), MAGE-C1, MAGE-C2, melan-A (e.g., melan-A according to accession number NM_00551 1), GP100 (e.g., GP100 according to accession number M77348), tyrosinase (e.g., tyrosinase according to accession number NM_000372), survivin (e.g., survivin according to accession number AF077350), CEA (e.g., CEA according to accession number NM_004363), Her-2/neu (e.g., Her-2/neu according to accession number M1 1 730), WT1 (e.g., WT1 according to accession number NM_000378). PRAME (e.g., PRAME according to accession number NM_0061 15), EGFRI (epidermal growth factor receptor 1) (e.g., EGFRI (epidermal growth factor receptor 1) according to accession number AF288738), MUC1, mucin-1 (e.g., mucin-1 according to accession number NM_002456), SEC61 G (e.g., SEC61 G according to accession number NM_014302), hTERT (e.g., hTERT accession number NM_198253), 5T4 (e.g., 5T4 according to accession number NM_006670), TRP-2 (e.g., TRP-2 according to accession number NM_001 922), STEAP1 (Six-transmembrane epithelial antigen of prostate 1), PSCA, PSA, PSMA, etc.

In some embodiments, the cancer antigen is selected from PCA, PSA, PSMA, STEAP, and optionally MUC-1, including fragments, variants, and derivatives thereof. In some embodiments, the cancer antigen selected from NY-ESO-1, MAGE-C1, MAGE-C2, survivin, 5T4, and optionally MUC-1, including fragments, variants, and derivatives thereof.

In some instances, cancer antigens encompass idiotypic antigens associated with a cancer or tumor disease, particularly lymphoma or a lymphoma associated disease, for example, wherein the idiotypic antigen is an immunoglobulin idiotype of a lymphoid blood cell or a T cell receptor idiotype of a lymphoid blood cell.

In some instances, the cancer antigen-specific T-cells are selected from one or more of chimeric antigen receptor (CAR)-modified T-cells (e.g., targeted against a cancer antigen), and T-cell Receptor (TCR)-modified T-cells, tumor infiltrating lymphocytes (TILs), and peptide-induced T-cells.

The skilled artisan will appreciate that the various cancer immunotherapy agents described herein can be combined with any one or more of the various anti-NRP2a antibodies (including antigen-binding fragments thereof) described herein, and used according to any one or more of the methods or compositions described herein.

Chemotherapeutic Agents. Certain embodiments employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), an anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The skilled artisan will appreciate that the various chemotherapeutic agents described herein can be combined with any one or more of the various anti-NRP2a antibodies (including antigen-binding fragments thereof) described herein, and used according to any one or more of the methods or compositions described herein.

Hormonal Therapeutic Agents. Certain embodiments employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, CCL21, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the IGF receptor (e.g., IGF-IR1) such as cixutumumab, dalotuzumab, figitumumab, ganitumab, istiratumab, and robatumumab; inhibitors of the vascular endothelial growth factor receptors 1, 2, or 3 (VEGFR1, VEGFR2 or VEGFR3) or their ligands such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; inhibitors of CCR7; and inhibitors of the PDGF receptor such as olaratumab and tovetumab.

The skilled artisan will appreciate that the various hormonal therapeutic agents described herein can be combined with any one or more of the various anti-NRP2a antibodies (including antigen-binding fragments thereof) described herein, and used according to any one or more of the methods or compositions described herein.

Kinase Inhibitors. Certain embodiments employ at least one kinase inhibitor, including tyrosine kinase inhibitors. Examples of kinase inhibitors include, without limitation, adavosertib, afatinib, aflibercept, axitinib, bevacizumab, bosutinib, cabozantinib, cetuximab, cobimetinib, crizotinib, dasatinib, entrectinib, erdafitinib, erlotinib, fostamatinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib. SU6656, tofacitinib, trastuzumab, vandetanib, and vemurafenib. Exemplary PI3 kinase inhibitors include alpelisib, buparlisib, copanlisib, CUDC-907, dactolisib, duvelisib, GNE-477, idelasib, IPI-549, LY294002, ME-401, perifosine, P1-103, pictilisib, PWT33597, RP6503, taselisib, umbralisib, voxtalisib, wortmannin, and XL 147 The skilled artisan will appreciate that the various kinase inhibitors described herein can be combined with any one or more of the various anti-NRP2a antibodies (including antigen-binding fragments thereof) described herein, and used according to any one or more of the methods or compositions described herein.

### Methods of Use and Therapeutic Compositions

Embodiments of the present disclosure relate in part to the discovery that certain human neuropilin 2 (NRP2) polypeptides, specifically NRP2a v1 and v2, contain unique juxtamembrane domain sequences that mediate binding interactions with NRP2a v1/v2 ligands such as CCL21 and/or CCR7. Such CCL21/CCR7-interacting domains are not found in human NRP2b v4 and NRP2b v5 polypeptides, and are only partially present in human NRP2a v3. Accordingly, antibodies that selectively bind to human NRP2a v1 and/or v2, relative to NRP2b v4 and NRP2b v5, and which inhibit or otherwise interfere with the binding between NRP2a v1/v2 and CCL21/CCR7, can be used to modulate downstream signaling events of these pathways. Such antibodies can be used as standalone therapies in the treatment of diseases, including NRP2-associated diseases, or in combination with other therapeutic agents as described herein.

Certain embodiments therefore include methods of treating, ameliorating the symptoms of, and/or reducing the progression of, a disease or condition in a subject in need thereof, comprising administering to the subject at least one antibody or antigen-binding fragment thereof that binds to a human NRP2a v and/or v2 polypeptide, as described herein. In some instances, an antibody or antigen-binding fragment thereof modulates (e.g., antagonizes) the binding between the NRP2a v1 and/or v2 polypeptide and CCL21 and/or CCR7. In some embodiments, the anti-NRP2a antibody, or antigen-binding fragment thereof, binds selectively to the NRP2a variant 1 and/or 2 isoforms, and does not substantially bind to the NRP2b v4 and v5 isoforms. In some embodiments, an antibody or antigen-binding fragment thereof modulates the signaling activities that result from the interaction between NRP2a v1 and/or v2 polypeptides and NRP2a ligands such as CCL21/CCR7.

Some embodiments include administering an anti-NRP2a antibody or antigen-binding fragment thereof in an amount and at a frequency sufficient to achieve a steady state concentration, or average circulating concentration, of anti-NRP2a antibody or antigen-binding fragment thereof of between about 1 nM and about 1 pM, between about 1 nM and about 100 nM, between about 1 nM and about 10 nM, or between about 1 nM and about 3 pM.

In certain embodiments, the disease or condition is an NRP2-associated disease or condition. In some embodiments, the NRP2-associated disease or condition is selected from one or more of cancer and diseases and pathways associated with cancer, including cancer cell growth, initiation, migration, adhesion, invasion, chemoresistance, and/or metastasis; diseases associated with inflammation, autoimmunity, and related inflammatory diseases, including diseases associated with inappropriate immune cell activation or migration such as graft versus host disease (GVHD); diseases associated with lymphatic development, lymphangiogenesis, and lymphatic damage, including, for example, edema, lymphedema, secondary lymphedema, inappropriate fat absorption and deposition, excess fat deposition, and vascular permeability; diseases associated with infections, including latent infections; diseases associated with allergic disorders/diseases, allergic responses, including, for example, chronic obstructive pulmonary disorder (COPD), neutrophilic asthma, antineutrophil cytoplasmic antibody (ANCA)-associated systemic vasculitis, systemic lupus erythematosus, rheumatoid arthritis, inflammasome-related diseases, and skin-related neutrophil-mediated diseases such as pyoderma gangrenosum; diseases associated with granulomatous inflammatory diseases, including sarcoidosis and granulomas; diseases associated with fibrosis including fibrotic diseases, fibrosis, endothelial to mesenchymal transition (EMT), and wound healing; diseases associated with inappropriate smooth muscle contractility, smooth muscle compensation and decompensation, and inappropriate vascular smooth muscle cell migration and adhesion; diseases associated with inappropriate autophagy, phagocytosis, and efferocytosis; diseases associated with neuronal diseases, peripheral nervous system remodeling, and pain perception; diseases associated with bone development and bone remodeling.

In some embodiments, the disease is a cancer. Certain embodiments thus include methods of treating ameliorating the symptoms of, or inhibiting the progression of, a cancer in a subject in need thereof, comprising administering to the subject at least one antibody or antigen-binding fragment thereof that specifically binds to a human NRP2a v1 and/or v2 poly peptide (an anti-NRP2a antibody), and which modulates (e.g., interferes with) binding of the human NRP2a polypeptide to a human NRP2a ligand (for example, an NRP2a ligand from Table N2 such as CCL21 and/or CCR7). Certain embodiments include reducing or preventing the re-emergence of a cancer in a subject in need thereof, for example, a metastatic cancer and/or a chemoresistant cancer, wherein administration of the therapeutic composition enables generation of an immune memory to the cancer.

In some-instances, an anti-NRP2a antibody or antigen-binding fragment thereof reduces cancer initiation, cancer cell migration, adhesion, or cancer cell metastasis by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control. In some-instances, an anti-NRP2a antibody or antigen-binding fragment thereof reduces cancer mediated lymphangiogenesis by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control. In some embodiments, an anti-NRP2a antibody or antigen-binding fragment thereof inhibits or reduces the rate of migration or motility of the cancer or a migratory cell (for example, cancer or immune cells isolated from a biopsy or other sample grown in vitro) by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control. In some embodiments, an anti-NRP2a antibody or antigen-binding fragment thereof reduces the invasiveness of the cancer (for example, cancer cells isolated from a biopsy or other sample grown in vitro) by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control.

In some embodiments, an anti-NRP2a antibody or antigen-binding fragment thereof enhances the susceptibility of the cancer to an additional agent (for example, chemotherapeutic agent, hormonal therapeutic agent, and or kinase inhibitor) by about or at least about 5, 10, 15, 20, 25, 30, 35, 40, 45, 50, 60, 70, 80, 90, 10, 200, 300, 400, 500, 600, 700, 800, 900, 1000, 2000% or more relative to the additional agent alone. In some embodiments, an anti-NRP2a antibody or antigen-binding fragment thereof enhances an anti-tumor and/or immunostimulatory activity of a cancer immunotherapy agent by about or at least about 5, 10, 15, 20, 25, 30, 35, 40, 45, 50, 60, 70, 80, 90, 100, 200, 300, 400, 500, 600, 700, 800, 900, 1000, 2000% or more, relative to the cancer immunotherapy agent alone.

Also include are combination therapies for treating cancers, including methods of treating ameliorating the symptoms of, or inhibiting the progression of, a cancer in a subject in need thereof, comprising administering to the subject at least one antibody or antigen-binding fragment thereof that specifically binds to a human NRP2a v1 and/or v2 polypeptide (an anti-NRP2a antibody) in combination with at least one additional agent, for example, a cancer immunotherapy agent, a chemotherapeutic agent, a hormonal therapeutic agent, and/or a kinase inhibitor. Exemplary cancer immunotherapy agents, chemotherapeutic agents, hormonal therapeutic agents, and kinase inhibitors are described elsewhere herein.

In some instances, an anti-NRP2a antibody, or antigen-binding fragment thereof, and the at least one additional agent are administered separately, for example, in separate therapeutic compositions and at the same or different times. In some embodiments, an anti-NRP2a antibody, or antigen-binding fragment thereof, and the at least one additional agent are administered as part of the same therapeutic composition, at the same time.

Particular methods employ one or more anti-NRP2a antibodies, or antigen-binding fragments thereof, as part of (i.e., in addition to) a combination therapy regimen. Exemplary combination regiments are provided in Table M1 below.

TABLE M1
Combination Therapy Regimens

| Cancer Type | Agents | Acronym |
|---|---|---|
| Breast Cancer | Cyclophosphamide, methotrexate, 5-fluorouracil, | CMF |
| | vinorelbine | |
| | Doxorubicin, cyclophosphamide | AC |
| Hodgkin's lymphoma | Docetaxel, doxorubicin, cyclophosphamide | TAC |
| | Doxorubicin, bleomycin, vinblastine, dacarbazine | ABVD |
| | Mustine, vincristine, procarbazine, prednisolone | MOPP |
| Non-Hodgkin's | Cyclophosphamide, doxorubicin, vincristine, | CHOP |
| lymphoma | prednisolone | |
| Germ cell tumor | Bleomycin, etoposide, cisplatin | BEP |
| Stomach cancer | Epirubicin, cisplatin, 5-fluorouracil | ECF |
| | Epirubicin, cisplatin, capecitabine | ECX |
| Bladder cancer | Methotrexate, vincristine, doxorubicin, cisplatin | MVAC |
| Lung cancer | Cyclophosphamide, doxorubicin, vincristine, | CAV |
| | vinorelbine | |
| Colorectal cancer | 5-fluorouracil, folinic acid, oxaliplatin | FOLFOX |
| Pancreatic Cancer | Leucovorin, fluorouracil, irinotecan (Camptosar), | FOLFIRINOX |
| | oxaliplatin | |
| | Gemcitabine, nabpaclitaxel | ABRAXANE |

In some embodiments, the methods and therapeutic compositions described herein (for example, anti-NRP2a antibody, alone or in combination with at least one additional agent) increase median survival time of a subject by 4 weeks, 5 weeks, 6 weeks, 7 weeks, 8 weeks, 9 weeks, 10 weeks, 15 weeks, 20 weeks, 25 weeks, 30 weeks. 40 weeks, or longer. In certain embodiments, the methods and therapeutic compositions described herein (for example, anti-NRP2a antibody, alone or in combination with at least one additional agent) increase median survival time of a subject by 1 year, 2 years, 3 years, or longer. In some embodiments, the methods and therapeutic compositions described herein (for example, anti-NRP2a antibody, alone or in combination with cancer immunotherapy agent) increase progression-free survival by 2 weeks, 3 weeks, 4 weeks. 5 weeks. 6 weeks, 7 weeks, 8 weeks, 9 weeks. 10 weeks or longer. In certain embodiments, the methods or therapeutic compositions described herein increase progression-free survival by 1 year, 2 years, 3 years, or longer.

In certain embodiments, the methods and therapeutic compositions described herein (for example, anti-NRP2a antibody, alone or in combination with at least one additional agent) are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein (for example, anti-NRP2a antibody, alone or in combination with at least one additional agent) are sufficient to result in stable disease. In certain embodiments, the methods and therapeutic compositions described herein (for example, anti-NRP2a antibody, alone or in combination with cancer immunotherapy agent) are sufficient to result in clinically relevant reduction in symptoms of a particular disease indication known to the skilled clinician.

In some embodiments, an anti-NRP2a antibody increases, complements, or otherwise enhances the anti-tumor and/or immunostimulatory activity of the cancer immunotherapy agent, relative to the cancer immunotherapy agent alone. In some embodiments, an anti-NRP2a antibody enhances the anti-tumor and/or immunostimulatory activity of the cancer immunotherapy agent by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to the cancer immunotherapy agent alone.

The methods and therapeutic compositions described herein can be used in the treatment of any variety of cancers or tumors. In some embodiments, the cancer is a primary cancer. i.e., a cancer growing at the anatomical site where tumor progression began and yielded a cancerous mass. In some embodiments, the cancer is a secondary or metastatic cancer, i.e., a cancer which has spread from the primary site or tissue of origin into one or more different sites or tissues. In some embodiments, the cancer expresses or overexpresses NRP2. In some embodiments, the subject or patient has a cancer selected from one or more of melanoma (e.g., metastatic melanoma), an epithelial or epithelial-derived tumor, pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma or HCC), sarcoma, B-cell malignancy, breast cancer (for example, estrogen receptor positive (ER+), estrogen receptor negative (ER−), Her2 positive (Her2+), Her2 negative (Her2−), or a combination thereof, e.g., ER+/Her2+, ER+/Her2−, ER−/Her2+, or ER−/Her2−; or “triple negative” breast cancer which is estrogen receptor-negative, progesterone receptor-negative, and HER2-negative), ovarian cancer, colorectal cancer, glioma (e.g., astrocytoma, oligodendroglioma, ependymoma, or a choroid plexus papilloma), glioblastoma multiforme (e.g., giant cell gliobastoma or a gliosarcoma), meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, stomach cancer, virus-induced tumors such as, for example, papilloma virus-induced carcinomas (e.g., cervical carcinoma, cervical cancer), adenocarcinomas, herpes virus-induced tumors (e.g., Burkitt's lymphoma, EBV-induced B-cell lymphoma), hepatitis B-induced tumors (hepatocellular carcinomas), HTLV-1-induced and HTLV-2-induced lymphomas, acoustic neuroma, lung cancers (e.g., lung carcinoma, bronchial carcinoma), small-cell lung carcinomas, pharyngeal cancer, anal carcinoma, glioblastoma, rectal carcinoma, lymphangia, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal cancer (e.g., oesophageal carcinoma), wart involvement, tumors of the small intestine, craniopharyngeomas, ovarian carcinoma, genital tumors, ovarian cancer (e.g., ovarian carcinoma), pancreatic cancer (e.g., pancreatic carcinoma), endometrial carcinoma, liver metastases, penile cancer, tongue cancer, gall bladder cancer, leukaemia, plasmocytoma, and lid tumor.

In some embodiments, as noted above, the cancer or tumor is a metastatic cancer, for example, a metastatic cancer that expresses NRP2a v1 and/or v2. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

In some embodiments, for example, where the cancer immunotherapy agent is a PD-1 or PD-L1 antagonist or inhibitor, the subject has one or more biomarkers (e.g., increased PD-1 or PD-L1 levels in cells such as cancer cells or cancer-specific CTLs) that make the suitable for PD-1 or PD-L1 inhibitor therapy. For instance, in some embodiments, the subject has increased fractions of programmed cell death 1 high/cytotoxic T lymphocyte-associated protein 4 high (e.g., PD-1^{hi}CTLA-4^{hi}) cells within a tumor-infiltrating CD8+ T cell subset (see, e.g., Daud et al., J Clin Invest. 126:3447-3452, 2016). As another example, in some embodiments, the subject has increased levels of Bim (B cell lymphoma 2-interacting (Bcl2-interacting) mediator) in circulating tumor-reactive (e.g., PD-1⁺CD11 a^{hi}CD8⁺) T cells, and optionally has metastatic melanoma (see, e.g., Dronca et al., JCI Insight. May 5; 1(6): e86014, 2016).

Certain specific combinations include an anti-NRP2a antibody and a PD-L1 antagonist or inhibitor, for example, atezolizumab (MPDL3280A), avelumab (MSB0010718C), and durvalumab (MEDI4736), for treating a cancer selected from one or more of colorectal cancer, melanoma, breast cancer, non-small-cell lung carcinoma, bladder cancer, and renal cell carcinoma.

Some specific combinations include an anti-NRP2a antibody and a PD-1 antagonist, for example, nivolumab, for treating a cancer selected from one or more of Hodgkin's lymphoma, melanoma, non-small cell lung cancer, hepatocellular carcinoma, renal cell carcinoma, and ovarian cancer.

Particular specific combinations include an anti-NRP2a antibody and a PD-1 antagonist, for example, pembrolizumab, for treating a cancer selected from one or more of melanoma, non-small cell lung cancer, small cell lung cancer, head and neck cancer, and urothelial cancer.

Certain specific combinations include an anti-NRP2a antibody and a CTLA-4 antagonist, for example, ipilimumab and tremelimumab, for treating a cancer selected from one or more of melanoma, prostate cancer, lung cancer, and bladder cancer.

Some specific combinations include an anti-NRP2a antibody and an IDO antagonist, for example, indoximod (NLG-8189), 1-methyl-tryptophan (1MT), β-Carboline (norharmane; 9H-pyrido[3,4-b]indole), rosmarinic acid, or epacadostat, for treating a cancer selected from one or more of metastatic breast cancer and brain cancer optionally Glioblastoma Multiforme, glioma, gliosarcoma or malignant brain tumor.

Certain specific combinations include an anti-NRP2a antibody and the cytokine INF-α for treating melanoma, Kaposi sarcoma, and hematologic cancers. Also included is the combination of an anti-NRP2a antibody and IL-2 (e.g., Aldesleukin) for treating metastatic kidney cancer or metastatic melanoma.

Some specific combinations include an anti-NRP2a antibody and a T-cell based adoptive immunotherapy, for example, comprising CAR-modified T-cells targeted against CD-19, for treating hematological cancers such as acute lymphoblastic leukemia (ALL), chronic lymphocytic leukemia (CLL), and B-cell neoplasms (see, e.g., Maude et al., 2015, supra; Lorentzen and Straten, Scand J Immunol. 82:307-19, 2015; and Ramos et al., Cancer J. 20:112-118, 2014).

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Certain embodiments include the use of the anti-NRP2a antibodies described herein to modulate lymphangiogenesis, and treat related lymphatic diseases or associated conditions such as lymphedema or tumor metastasis. The lymphatic system consists of networks of interconnected capillaries, collecting vessels and lymph nodes that absorb, collect and transport the fluid and protein filtered from the blood vascular system. This system provides a critical homeostatic function; in humans, lymphatic vessels return >4 liters of fluid and a substantial amount of protein per day back into the great veins of the neck.

Lymphatic vascular dysfunction (lymphedema) results in the accumulation of excess fluid (edema) in the interstitium. Although lymphedema is typically not life-threatening, it has serious health consequences, including pain, immobility, fibrosis, inflammation, adipose tissue accumulation, and tissue damage. Because the lymphatic system is also a critical component of immune responses, lymphedema is typically accompanied by an increased risk of infection and other immune system problems.

Lymphangiogenesis is the formation of new lymphatic vessels from preexisting lymphatic vessels and is associated with diverse pathological conditions including metastatic dissemination, graft rejection (e.g., cornea, kidney and heart), type 2 diabetes, obesity, hypertension, and lymphedema (See, e.g. Alitalo et al. Nature 438:946-953, 2005; Karaman et al. J Clin Invest 124:922-928, 2014; Kim et al., J Clin Invest 124:936-942, 2014; Maby-El Hajjami et al., Histochem Cell Biol 130:1063-107, 2008; Machnik et al., Nat Med 15:545-552; Mortimer et al., 2014. J Clin Invest 124:915-921; Skobe et al., 2009. Nat Med 15:993-994).

Lymphatic vessel invasion in and around a primary tumor compared to invasion of blood vessels is a prognostic marker of the aggressiveness of various types of cancers. Growth of lymphatic vessels is also involved in graft rejection (Dietrich, T., et al., J Immunol 184:535-539, 2010. Hall et al., Arch Otolaryngol Head Neck Surg 129:716-719, 2003; Maula et al., Cancer Res 63:1920-1926, 2003; Miyata et al., J Urol 176:348-353, 2006; Saad et al., Mod Pathol 19:1317-1323, 2006; Schoppmann et al., Ann Surg 240:306-312, 2004; Zeng et al., Prostate 65:222-230, 2005).

Anti-lymphangiogenic agents are useful, for example, for treating debilitating diseases of the eye, where the growth of lymphatic vessels is the major reason of corneal graft rejection and a major contributor to neovascularization associated with age related macular degeneration (Dietrich, T., et al., J Immunol 184:535-539, 2010). In particular, penetrating keratoplasty is the most common form of solid tissue transplantation, with approximately 40,000 corneal transplantations performed each year in the United States. The success rate of penetrating keratoplasty is as high as 90% for uncomplicated first grafts performed in avascular low-risk beds. However, the rejection rate of the corneal grafts placed in high-risk vascularized host beds is extremely high (70% to 90%). Thus certain embodiments include anti-NRP2a antibodies for inhibiting lymphangiogenesis and thereby promoting graft survival and inhibiting neovascularization

Anti-lymphangiogenesis drugs are useful also for treatment of dry eye disease. Significant upregulation of pro-lymphangiogenic factors (e.g., VEGF-C, VEGF-D, and VEGFR-3) and selective growth of lymphatic vessels without concurrent growth of blood vessels has been demonstrated in corneas with dry eye disease (Goyal et al., Arch Ophthalmol 128:819-824, 2010). Dry eye disease is an immune-mediated disorder affecting about 5 million Americans. It severely impacts the vision-related quality of life and the symptoms can be debilitating. The current therapeutic options for dry eye disease are limited, mostly palliative, and expensive. Some embodiments thus include anti-NRP2a antibodies as lymphangiogenesis inhibitors for treatment of dry eye disease.

Metastases are responsible for the vast majority (90%) of deaths from solid tumors (Gupta and Massague, Cell 127, 679-695, 2006). The complex process of metastasis involves a series of distinct steps including detachment of tumor cells from the primary tumor, intravasation of tumor cells into lymphatic or blood vessels, and extravasation and growth of tumor cells in secondary sites. Analysis of regional lymph nodes in many tumor types suggests that the lymphatic vasculature is an important route for the dissemination of human cancers. Furthermore, in almost all carcinomas, the presence of tumor cells in lymph nodes is the most important adverse prognostic factor. While it was previously thought that such metastases exclusively involved passage of malignant cells along preexisting lymphatic vessels near tumors, recent experimental studies and clinicopathological reports (See. e.g., Achen et al., Br J Cancer 94, 1355-1360, 2006 and Nathanson, Cancer 98,413-423, 2003) suggest that lymphangiogenesis can be induced by solid tumors and can promote tumor spread. Some embodiments thus include the use anti-NRP2a antibodies for targeting lymphatics and lymphangiogenesis as a therapeutic strategy to restrict the development of cancer metastasis.

Accordingly, the methods and compositions described herein can be used to inhibit the activities of pro-lymphangiogenic factors and thereby treat graft rejection, dry-eye disease, tumor metastasis, lymphedema, and related inflammatory conditions.

Some embodiments include the use of the anti-NRP2 antibodies described herein to modulate smooth muscle contractility, and treat related conditions. Reduced smooth muscle (SM) contractility in the bladder can stem from numerous etiologies including partial obstruction secondary to benign prostatic hyperplasia (BPH), posterior urethral valves, diabetes mellitus, multiple sclerosis, spinal cord injury, or idiopathic causes. (See, e.g., Drake et al., Nat Rev Urol. I1(8):454-464, 2014). In conditions such as BPH or posterior urethral valves, the bladder contracts against an obstructed outlet. The initial response is adaptive, involving a compensatory phase of SM hypertrophy that enables increased force generation to overcome the increased outlet resistance. When the demand outstrips the adaptive capability of the bladder, contractile performance becomes less efficient, residual volumes increase, and the bladder remodels, ultimately leading to a loss of detrusor contractility as the bladder decompensates. (See, e.g., Zderic et al., J Cell Mol Med. 16(2):203-217, 2012). The prevalence of underactive detrusor function is reported to be as high as 48% in adults (Osman et al., Eur Urol; 65(2):389-398, 2014). Furthermore, existing pharmacological treatments for restoration of SM contraction such as muscarinic agonists or cholinesterase inhibitors have shown limited efficacy and adverse effects (Barendrecht et al., BJU Int. 99(4):749-752, 2007).

Recent studies have identified bladder smooth muscle as a major site of NRP2 expression, demonstrated the inhibition of RhoA and cytoskeletal stiffness, and observed increased contractility of bladder SM strips from mice with ubiquitous or smooth muscle-specific deletion of NRP2 in vivo, when compared with tissues from NRP2-intact littermate controls (See, e.g., Bielenberg et al., Am. J. Pathol. 181 548-559, 2012; and Vasquez et al., JCI Insight 2(3) e90617, 2017).

Furthermore, recent studies have shown that targeting NRP2 in bladders undergoing decompensation has the potential to restore contractility in spite of ongoing obstruction. (Vasquez et al., JCI Insight 2(3) e90617, 2017). These findings argue that the NRP2 axis represents a potentially novel pharmacologic target for restoration of SM contractility in partial bladder outlet obstruction syndromes, and provide an important therapeutic opportunity for the development of antibody-based modulators of NRP2 function.

Pharmacological management of diminished detrusor contractility to date has focused on stimulation of parasympathetic activity to enhance bladder contractility and reduction of outflow resistance to facilitate bladder emptying (Chancellor et al., Urology 72(5) 966-967, 2008). However, analysis of 10 randomized clinical trials of parasympathomimetic drugs in patients with poorly contractile bladders, revealed either a worsening of symptoms or a lack of significant improvement (Barendrecht et al., BJU Int. 99(4) 749-752, 2007). The increase in contractility following NRP2 deletion in the decompensating bladder suggests that NRP2 may be a useful target to mitigate reduced detrusor contractility under conditions of chronic obstruction.

Given the role of NRP2 in this process, certain embodiments include the use of anti-NRP2 antibodies described herein to modulate smooth muscle contractility, including for example, in the treatment of reduced smooth muscle (SM) contractility in the bladder, and more specifically syndromes associated with partial bladder outlet obstruction syndromes. Some embodiments thus include methods of treating, ameliorating the symptoms of, or inhibiting the progression of partial bladder outlet obstruction syndromes in a subject in need thereof, comprising administering to the subject at least one antibody, or antigen-binding fragment thereof, as described herein, which specifically binds to a human NRP2a polypeptide. Certain embodiments include methods of modulating (e.g., increasing, reducing) smooth muscle contractility in a subject in need thereof, comprising administering to the subject an anti-NRP2a antibody, or antigen-binding fragment thereof, as described herein. Certain embodiments include treating, ameliorating the symptoms of, and/or reducing the progression of, reduced smooth muscle contractility in a subject in need thereof, comprising administering to the subject an anti-NRP2a antibody, or antigen-binding fragment thereof, as described herein.

NRP2 expression has been associated with fibrosis development. Some embodiments thus include use of the anti-NRP2a antibodies described herein for treating fibrosis, for example, tissue fibrosis. Examples of tissue fibrosis includes fibrosis selected from the group consisting of hepatic fibrosis, renal fibrosis, pulmonary fibrosis, skin fibrosis, cardiovascular fibrosis, and gastrointestinal fibrosis, among other fibrous diseases. Examples of hepatic fibrosis include hepatic cirrhosis, ischemic reperfusion, post-hepatic transplant disorder, necrotic hepatitis, hepatitis B, hepatitis C, primary biliary cirrhosis, and primary sclerosing cholangitis. In some aspects, hepatic cirrhosis is related to induction by alcohol, drugs, and/or other chemicals. Examples of renal fibrosis include proliferative glomerulonephritis, sclerotic glomerulonephritis, nephrogenic fibrosing dermopathy, diabetic nephropathy, renal tubule interstitial fibrosis, and focal segmental glomerulosclerosis. Examples of pulmonary fibrosis include pulmonary interstitial fibrosis, drug induced sarcoidosis, pulmonary fibrosis, idiopathic pulmonary fibrosis, asthma, chronic obstructive pulmonary disease, diffuse pulmonary alveolar injury disease, pulmonary hypertension, and neonatal bronchopulmonary dysplasia. Examples of skin fibrosis include scleroderma, keloid scarring, psoriasis, hypertrophic scarring, and pseudo scleroderma. Examples of cardiovascular fibrosis include atherosclerosis, coronary restenosis, congestive cardiomyopathy, heart failure, cardiac transplantation, and myocardial fibrosis. Examples of gastrointestinal fibrosis includes collagenous colitis, villous atrophy, crypt hyperplasia, polyp formation, fibrosis of Crohn's disease, gastric ulcer healing, and post-abdominal adhesion surgery scar. Also included are fibrotic conditions arising from bone-related fibrosing disease and rheumatoid pannus formation.

Methods for identifying subjects with one or more of the diseases or conditions described herein are known in the art. In some embodiments, the subject has, and/or is selected for treatment based on having, a disease associated with increased levels or expression of at least one NRP2a ligand such as CCL21 and/or CCR7 and/or a coding mRNA thereof relative to a healthy control. For instance, in some embodiments, the levels of the at least one NRP2a ligand in the diseases subject, cells, or tissue are about or at least about 1.5, 2, 3, 4, 5, 6, 7, 8, 9, 10, 15, 20, 30, 40, 50, 60, 70, 80, 90, 100, 1000 or more times the levels of the at least one NRP2a ligand in a healthy control. In some embodiments, the subject has, and/or is selected for treatment based on having, a cancer which has increased levels or expression of at least one NRP2a ligand and/or a coding mRNA thereof relative to a non-cancerous control cell or tissue. For instance, in some embodiments, the levels of the at least one NRP2a ligand in the cancer cells or tissue are about or at least about 1.5, 2, 3, 4, 5, 6, 7, 8, 9, 10, 15, 20, 30, 40, 50, 60, 70, 80, 90, 100, 1000 or more times the levels of the NRP2a ligand in a non-cancerous control or standard. Thus, certain embodiments include methods of selecting a subject for treatment, comprising (i) detecting increased expression levels of at least one NRP2a ligand and/or coding mRNA in the subject relative to a control or reference, and (ii) administering to the subject a therapeutic composition comprising at least one anti-NRP2a-antibody or antigen-binding fragment thereof, as described herein. In particular embodiments, the NRP2a ligand is CCL21 and/or CCR7.

In some embodiments, the subject has, and/or is selected for treatment based on having, increased circulating or serum levels of a NRP2a polypeptide, for example, a soluble NRP2a polypeptide (selected, for example, from Table N1), relative to the levels of a healthy or matched control population of subject(s). For instance, in certain embodiments, the circulating or serum levels are about or at least about 10, 20, 30, 50, 100, 200, 300, 400, 500, 600, 700, 800, 900, 1000, 1100, 1200, 1300, 1400, 1500, 1600, 1700, 1800, 1900, 2000, 3000, 4000, 5000 pM of the soluble NRP2a polypeptide, or the circulating or serum levels are about 30-50, 50-100, 100-2000, 200-2000, 300-2000, 400-2000, 500-2000, 600-2000, 700-2000, 800-2000, 900-2000, 1000-2000, 2000-3000, 3000-4000, 4000-5000 pM of the soluble NRP2a polypeptide.

In certain embodiments, the subject has, and/or is selected for treatment based on having, a disease associated with increased levels or expression of an NRP2a polypeptide (optionally selected from Table N1) and/or a coding mRNA thereof relative to a healthy control (e.g., an NRP2-associated disease). In some embodiments, the NRP2a polypeptide is an NRP2a variant 1 or variant 2 isoform, or a fragment thereof. For example, in certain embodiments, the levels of the NRP2a polypeptide in the diseased subject, cells, or tissue are about or at least about 1.5, 2, 3, 4, 5, 6, 7, 8, 9, 10, 15, 20, 30, 40, 50, 60, 70, 80, 90, 100 or more times the levels of NRP2a polypeptide in a healthy control. In some embodiments, the subject has, and/or is selected for treatment based on having, a cancer which has increased levels or expression of a NRP2a polypeptide (selected, for example, from Table N1) and/or a coding mRNA thereof relative to a control cell or tissue, optionally relative to a non-cancerous cell or tissue of the same type as the cancer. For instance, in some embodiments, the levels of the NRP2a poly peptide in the cancer cells or tissue are about or at least about 1.5, 2, 3, 4, 5, 6, 7, 8, 9, 10, 15, 20, 30, 40, 50, 60, 70, 80, 90, 100 or more times the levels of NRP2a polypeptide in a non-cancerous control or standard. Some embodiments thus include methods of selecting a subject for treatment, comprising (i) detecting increased expression levels of a NRP2a polypeptide and/or a coding mRNA thereof in the subject relative to a control or reference, and (ii) administering to the subject a therapeutic composition comprising at least one anti-NRP2a antibody or antigen-binding fragment thereof, as described herein.

In some embodiments, the subject has, and/or is selected for treatment based on having, a disease associated with increased levels or expression of NRP2a (e.g., variants 1 and/or 2 of Table N1), or an altered ratio of NRP2a:NRP2b expression, relative to a healthy control or matched control standard or population of subject(s). In some embodiments, the subject has significantly higher expression or levels of NRP2a relative to a healthy control or matched control standard or population of subject(s). In some embodiments, the levels of NRP2a are increased by about or at least about 10%, 20%, 30%, 40%, 50%, 100%, 200%, 300%, 400%, 500%, 600%, 700%, 800%, 900%, 1000% compared to a healthy control or matched control standard or population of subject(s). Certain embodiments therefore include methods of selecting a subject for cancer treatment, comprising (i) detecting increased expression levels of NRP2a in the subject relative to a control or reference, and (ii) administering to the subject a therapeutic composition comprising an anti-NRP2a-antibody or antigen-binding fragment thereof, as described herein.

In some embodiments, the healthy control or matched control standard or population of subject(s) comprises average ranges for age-matched samples of diseased or non-diseased cells or tissue of the same type, which comprise specific characteristics such as drug resistance, metastatic potential, aggressiveness, genetic signature (e.g., p53 mutations, PTEN deletion, IGFR expression), and/or expression patterns.

For in vivo use, as noted above, for the treatment of human or non-human mammalian disease or testing, the agents described herein are generally incorporated into one or more therapeutic or pharmaceutical compositions prior to administration, including veterinary therapeutic compositions.

Thus, certain embodiments relate to therapeutic compositions that comprise at least one antibody or antigen-binding fragment thereof that specifically binds to a human NRP2a v1 and/or v2 polypeptide, as described herein. In some instances, a therapeutic or pharmaceutical composition comprises one or more of the agents described herein in combination with a pharmaceutically- or physiologically-acceptable carrier or excipient. Certain therapeutic compositions further comprise at least one cancer immunotherapy agent, as described herein.

Some therapeutic compositions comprise (and certain methods utilize) only one anti-NRP2a antibody or antigen-binding fragment thereof. Certain therapeutic compositions comprise (and certain methods utilize) a mixture of at least two, three, four, or five different anti-NRP2a antibodies or antigen-binding fragments thereof.

For instance, certain therapeutic compositions comprise at least two anti-NRP2 antibodies, including a first antibody or antigen-binding fragment thereof that specifically binds to at least one first epitope of a human NRP2a polypeptide, and a second antibody or antigen-binding fragment thereof that specifically binds to at least one second epitope of the same or different human NRP2 polypeptide, wherein the at least one first epitope differs from the at least one second epitope. In some embodiments, the first and the second antibody or antigen-binding fragment thereof specifically and non-competitively bind to the same domain of the NRP2 polypeptide. In some embodiments, the first anti-NRP2 antibody or antigen-binding fragment thereof binds selectively to a first epitope that is specific to the NRP2a isoform (e.g., variants 1 and/or 2 of Table N1), and the second anti-NRP2 antibody or antigen-binding fragment thereof selectively binds to a second epitope which is specific to the NRP2b isoform (e.g., variants 4 and/or 5 of Table N1), or common to both NRP2a and NRP2b isoforms (e.g., located in the common, surface exposed a1,a2, b1, b2 or c domains shared by full length NRP2a and NRP2b).

In some embodiments, the first and the second antibody or antigen-binding fragment thereof specifically and non-competitively bind to different domains of the NRP2 polypeptide. In some embodiments, the first antibody modulates the signaling activity between the NRP2a polypeptide and the at least one NRP2a ligand, for example, CCL21 and/or CCR7.

In some embodiments, the first and the second antibody or antigen-binding fragments thereof are both blocking antibodies, for example, for at least two different NRP2 ligands. In some embodiments, the first and the second antibody or antigen-binding fragments thereof are both partial-blocking antibodies, for example, for at least two different NRP2 ligands. In some instances, the first and the second antibodies or antigen-binding fragments thereof are both non-blocking antibodies, for example, with respect to at least two different NRP2 ligands.

In some instances, the first antibody or antigen-binding fragment thereof is a blocking antibody and the second antibody or antigen-binding fragment thereof is a partial-blocking antibody. In certain instances, the first antibody or antigen-binding fragment thereof is a blocking antibody and the second antibody or antigen-binding fragment thereof is a non-blocking antibody.

In some embodiments, the first and the second antibodies or antigen-binding fragments thereof both comprise an IgG Fc domain with high effector function in humans, for example, an IgG1 or IgG3 Fc domain. In some embodiments, the first and the second antibodies or antigen-binding fragments thereof comprise an IgG Fc domain with low effector function in humans, for example, an IgG2 or IgG4 Fc domain.

In some instances, the first antibody or antigen-binding fragment thereof comprises an IgG Fc domain with high effector function in humans, for example, an IgG1 or IgG3 Fc domain, and the second antibody or antigen-binding fragment thereof comprises an IgG Fc domain with low effector function in humans, for example, an IgG2 or IgG4 Fc domain.

In particular embodiments, the therapeutic composition comprising the agents such as anti-NRP2a antibodies or other polypeptide agents is substantially pure on a protein basis or a weight-weight basis, for example, the composition has a purity of at least about 80%, 85%, 90%, 95%, 98%, or 99% on a protein basis or a weight-weight basis.

In some embodiments, the antibodies (e.g., anti-NRP2a antibodies) or other polypeptide agents provided herein do not form aggregates, have a desired solubility, and/or have an immunogenicity profile that is suitable for use in humans, as described herein and known in the art. Thus, in some embodiments, the therapeutic composition comprising a polypeptide agent (for example, an anti-NRP2a antibody) is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise a polypeptide agent (e.g., an antibody such as an anti-NRP2a antibody) that is at least about 50%, about 60%, about 70%, about 80%, about 90% or about 95% monodisperse with respect to its apparent molecular mass.

In some embodiments, polypeptide agents such as antibodies (e.g., anti-NRP2a antibodies) are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml. 9 mg/ml. 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, intraocular, subcutaneous, direct instillation into the bladder, or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intraocular, intradermal, intramuscular, subcutaneous, installation into the bladder, or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as poly vinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, ocular, rectal, vaginal, and intranasal. The term parenteral as used herein includes subcutaneous injections, intravenous, instillation into the bladder, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, gel, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, gel, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral, intraocular, or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

The therapeutic or pharmaceutical compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration: the rate of excretion; the drug combination: the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., ˜3.5 g); more preferably a therapeutically effective dose is (for a 70 kg mammal) from about 1 mg/kg (i.e., ˜70 mg) to about 25 mg/kg (i.e., 1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains an anti-NRP2a antibody and an additional therapeutic agent (e.g., immunotherapy agent, chemotherapeutic agent, hormonal therapeutic agent, kinase inhibitor), as well as administration of compositions comprising an anti-NRP2a antibody and an additional therapeutic agent in its own separate pharmaceutical dosage formulation. For example, an anti-NRP2a antibody as described herein and additional therapeutic agent can be administered to the subject together in a single oral dosage composition such as a tablet or capsule, or each agent administered in separate oral dosage formulations. Similarly, an anti-NRP2a antibody as described herein and additional therapeutic agent can be administered to the subject together in a single parenteral dosage composition such as in a saline solution or other physiologically acceptable solution, or each agent administered in separate parenteral dosage formulations. As another example, for cell-based therapies, an anti-NRP2a antibody can be mixed with the cells prior to administration, administered as part of a separate composition, or both. Where separate dosage formulations are used, the compositions can be administered at essentially the same time, i.e., concurrently, or at separately staggered times, i.e., sequentially and in any order, combination therapy is understood to include all these regimens.

Also included are patient care kits, comprising (a) at least one antibody or antigen-binding fragment thereof that specifically binds to a human NRP2a variant 1 and/or variant 2 polypeptide (an anti-NRP2a antibody), as described herein; and optionally (b) at least one additional therapeutic agent (e.g., immunotherapy agent, chemotherapeutic agent, hormonal therapeutic agent, kinase inhibitor). In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an anti-NRP2a antibody and optionally at least one additional therapeutic agent. For example, the kit includes a plurality of syringes, ampules, foil packets, or blister packs, each containing a single unit dose of an anti-NRP2a antibody and optionally at least one additional therapeutic agent. The containers of the kits can be air tight, waterproof (e.g., impermeable to changes in moisture or evaporation), and/or light-tight.

The patient care kit optionally includes a device suitable for administration of the composition. e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

### Bioassays and Analytical Assays for Drug Release Assays and Product Specifications, Diagnostics, and Reagents

Also included are bioassays that relate to anti-NRP2a antibodies and related agents such as therapeutic and diagnostic reagents. Examples include bioassays and analytical assays that measure purity, biological activity, affinity, solubility, pH, endotoxin levels, among others, many of which are described herein. Also included are assays that establish dose response curves and/or provide one or more bases for comparison between different batches of antibody. Batch comparisons can be based on any one or more of chemical characterization, biological characterization, and clinical characterization. Also included are methods of evaluating the potency, stability, pharmacokinetics, and immunogenicity of a selected antibody. Among other uses, these and other methods can be used for lot releasing testing of biologic or chemical agents, including anti-NRP2a antibodies, described herein.

Certain embodiments include the use of bioaffinity assays. Such assays can be used to assess the binding affinity, for example, between an anti-NRP2a antibody and at least one NRP2 ligand (for example, an NRP2a ligand such as CCL21 and/or CCR7), including its ability to interfere with the interaction between a human NRP2a polypeptide and the at least one NRP2 ligand, or other cellular binding partner. Certain exemplary binding affinity assays may utilize ELISA assays or protein-protein interaction assays such as the NanoBiT® Protein:Protein Interaction System (Promega), among other protein complementation assays and approaches as described herein and known in the art. Certain assays utilize high-performance receptor binding chromatography (see. e.g., Roswall et al., Biologicals. 24:25-39, 1996). Other exemplary binding affinity assays may utilize surface plasmon resonance (SPR)-based technologies. Examples include BIACore technologies, certain of which integrate SPR technology with a microfluidics system to monitor molecular interactions in real time at concentrations ranging from pM to mM. Also included are KINEXA™ assays, which provide accurate measurements of binding specificity, binding affinity, and binding kinetics/rate constants.

Certain embodiments relate to immunoassays for evaluating or optimizing the immunogenicity of anti-NRP2a antibodies. Examples include ex vivo human cellular assays and in vitro immuno-enzymatic assays to provide useful information on the immunogenic potential of a therapeutic protein. Ex vivo cell-response assays can be used, for example, to reproduce the cellular co-operation between antigen-presenting cells (APCs) and T-cells, and thereby measure T-cells activation after contact with a protein of interest. Certain in vitro enzymatic assays may utilize a collection of recombinant HLA-DR molecules that cover a significant portion of a relevant human population, and may include automated immuno-enzymatic assays for testing the binding of peptides (stemming from the fragmentation of the therapeutic protein) with the HLA-DR molecules. Also included are methods of reducing the immunogenicity of a selected protein, such as by using these and related methods to identify and then remove or alter one or more T-cell epitopes from an anti-NRP2a antibody.

Also included are biological release assays (e.g., cell-based assays) for measuring parameters such as specific biological activities, binding characteristics, NRP2 receptor dimerization and heterodimerization, or changes in signal transduction, receptor localization, internalization or tempospatial kinetics of NRP2 polypeptides, and or other parameters such as plasticity, growth, and/or cytotoxicity. Certain specific biological assays include, for example, cell-based assays that utilize a cellular binding partner, for example, a cell-surface receptor such as a NRP2a v1 and/or v2 polypeptide, a different NRP2 polypeptide (see Table N1), and/or at least one NRP2 ligand such as CCL21 and/or CCR7, presented on the cell surface, which is either endogenously, or recombinantly expressed on the cell surface), which is functionally coupled to a readout, such as a fluorescent or luminescent indicator of NRP2 or NRP2 ligand binding, or functional activity, as described herein.

For instance, specific embodiments include a cell that either endogenously or recombinantly expresses a human NRP2 polypeptide (e.g., NRP2a v1 and/or v2 polypeptide on the cell surface, or other NRP2 polypeptide), which allows assessment of the ability of anti-NRP2a antibody to bind one or more of the NRP2 polypeptides described herein. In some embodiments, the anti-NRP2a antibody and/or the NRP2 poly peptide is/are functionally coupled to a readout or indicator, such as a fluorescent or luminescent indicator to measure the binding and/or biological activity of the NRP2 polypeptide. Exemplary protein-protein interaction assays which are capable of monitoring the interaction of a NRP2a polypeptide with a NRP2a ligand in response to an anti-NRP2a antibody include split sensor systems such as the NanoBiT® Protein:Protein Interaction System (Promega). In some embodiments, the cell also expresses at least one NRP2 ligand (for example, an NRP2a ligand from Table N2 such as CCR7), wherein the at least one NRP2 ligand is coupled to a readout or indicator, such as a fluorescent or luminescent indicator of binding and/or biological activity of the at least one NRP2 ligand to an NRP2a polypeptide.

Also included are in vivo biological assays to characterize the pharmacokinetics of an anti-NRP2a antibody, typically utilizing engineered, or wild type mice, rat, monkey or other mammal (see. e.g., Lee et al., The Journal of Pharmacology. 281:1431-1439, 1997). Examples of cytotoxicity-based biological assays include release assays (e.g., chromium or europium release assays to measure apoptosis; see. e.g., von Zons et al., Clin Diagn Lab Immunol. 4:202-207, 1997), among others, which can assess the cytotoxicity of anti-NRP2a antibodies, whether for establishing dose response curves, batch testing, or other properties related to approval by various regulatory agencies, such as the Food and Drug Administration (FDA). Also included are assays for evaluating the effects of an anti-NRP2a antibody on immune cells, for example, dendritic cells.

Certain embodiments include an assay system, comprising a single monoclonal anti-NRP2a antibody and at least one human NRP2a v1/and or v2 polypeptide, wherein the anti-NRP2a antibody binds to the NRP2a polypeptide. In some instances, the at least one antibody comprises an IgG4 Fc domain.

Also included are testing material(s), comprising a purified NRP2 polypeptide (e.g., NRP2a v1 and/or v2 polypeptide), wherein said purified NRP2 polypeptide is bound to a solid substrate in a manner that enables antibody binding detection.

Such assays and materials can be used, for example, to develop a dose response curve for a selected anti-NRP2a antibody, and/or to compare the dose response curve of different batches of proteins or other agents. A dose-response curve is an X-Y graph that relates the magnitude of a stressor to the response of a receptor, such as an NRP2-NRP2 ligand (for example, an NRP2a ligand from Table N2) interaction; the response may be a physiological or biochemical response, such as a non-canonical biological activity in a cell in vitro or in a cell or tissue in vivo, a therapeutically effective amount as measured in vivo (e.g., as measured by EC₅₀), or death, whether measured in vitro or in vivo (e.g., cell death, organismal death). Death is usually indicated as an LD₅₀, a statistically-derived dose that is lethal to 50% of a modeled population, though it can be indicated by LC₀₁ (lethal dose for 1% of the animal test population). LC₁₀₀ (lethal dose for 100% of the animal test population), or LC_{LO} (lowest dose causing lethality). Almost any desired effect or endpoint can be characterized in this manner.

The measured dose of a response curve is typically plotted on the X axis and the response is plotted on the Y axis. More typically, the logarithm of the dose is plotted on the X axis, most often generating a sigmoidal curve with the steepest portion in the middle. The No Observable Effect Level (NOEL) refers to the lowest experimental dose for which no measurable effect is observed, and the threshold dose refers to the first point along the graph that indicates a response above zero. As a general rule, stronger drugs generate steeper dose response curves. For many drugs, the desired effects are found at doses slightly greater than the threshold dose, often because lower doses are relatively ineffective and higher doses lead to undesired side effects. For in vivo generated dose response curves, a curve can be characterized by values such as pg/kg, mg/kg, or g/kg of body-weight, if desired.

For batch comparisons, it can be useful to calculate the coefficient of variation (CV) between different dose response curves of different batches (e.g., between different batches of anti-NRP2a antibody), in part because the CV allows comparison between data sets with different units or different means. For instance, in certain exemplary embodiments, two or three or more different batches of anti-NRP2a antibodies or other agents have a CV between them of less than about 30%, 20%, 15%, 14%, 13%, 12%, 11%, 10%, 9%, 8%, 7%, 6%, 5%, 4%, 3%, 2%, or 1% for a 4, 5, 6, 7, or 8 point dose curve. In certain embodiments, the dose response curve is measured in a cell-based assay, and its readout relates to an increase or a decrease in a selected activity of an anti-NRP2a antibody. In certain embodiments, the dose response curve is measured in a cell release assay or animal model (e.g., mouse model), and its readout relates to cell death or animal death. Other variations will be apparent to persons skilled in the art.

### Expression and Purification Systems

Certain embodiments include methods and related compositions for expressing and purifying an anti-NRP2a antibody or other polypeptide-based agent described herein. Such recombinant anti-NRP2a antibodies can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, anti-NRP2a antibodies may be prepared by a procedure including one or more of the steps of: (a) preparing a construct comprising a polynucleotide sequences that encode an anti-NRP2a antibody heavy and light chain and that are operably linked to a regulatory element; (b) introducing the constructs into a host cell; (c) culturing the host cell to express an anti-NRP2a antibody; and (d) isolating an anti-NRP2a antibody from the host cell.

Anti-NRP2a antibody polynucleotides are described elsewhere herein. In order to express a desired polypeptide, a nucleotide sequence encoding an anti-NRP2a antibody, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector-enhancers, promoters, 5′ and 3′ untranslated regions-which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed poly peptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like, pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments may employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in Ion and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS·TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326.681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see. e.g., Hobbs in McGraw Hill. Yearbook of Science and Technology. pp. 191-196 (1992)).

An insect system may also be used to express a polypeptide of interest. For example, in one such system. Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the poly peptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., PNAS. U.S.A. 91:3224-3227 (1994)). Also included are baculovirus expression systems, including those that utilize SF9, SF21, and T. ni cells (see, e.g., Murphy and Piwnica-Worms, Cuff Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2), canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562. ATCC CCL51); TR1 cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for antibody production, see, e.g., Yazaki and Wu. Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners. 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., PNAS USA. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinothricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:12941, 2009)). Also included are high-throughput cell-free protein expression systems (see, e.g., Sitaraman et al., Methods Mol Biol. 498:229-44, 2009). These and related embodiments can be used, for example, to generate microarray s of anti-NRP2a antibodies which can then be used for screening libraries to identify antibodies and antigen-binding domains that interact with the NRP2 polypeptide(s) of interest.

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. Certain specific embodiments utilize serum free cell expression systems. Examples include HEK293 cells and CHO cells that can be grown on serum free medium (see, e.g., Rosser et al., Protein Expr. Purif. 40:237-43, 2005; and U.S. Pat. No. 6,210,922).

An antibody, or antigen-binding fragment thereof, produced by a recombinant cell may be secreted or contained intracellularly depending on the sequence and/or the vector used. As will be understood by those of skill in the art, expression vectors containing polynucleotides may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin. FLAG tags, poly-histidine tags (e.g., 6×His), cMyc tags. V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating anti-NRP2a antibodies and antigen-binding fragments thereof, and composition comprising concentrated soluble proteins. In different aspects such concentrated solutions of anti-NRP2a antibodies may comprise proteins at a concentration of about 5 mg/mL; or about 8 mg/mL; or about 10 mg/mL; about 15 mg/mL; or about 20 mg/mL.

In some aspects, such compositions may be substantially monodisperse, meaning that an at least one anti-NRP2a antibody exists primarily (i.e., at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, the reagents, anti-NRP2a antibodies, or related agents have a purity of at least about 90%, as measured according to routine techniques in the art. In certain embodiments, such as diagnostic compositions or certain therapeutic compositions, an anti-NRP2a antibody composition has a purity of at least about 95%. In specific embodiments, such as therapeutic or pharmaceutical compositions, an anti-NRP2a antibody composition has a purity of at least about 97% or 98% or 99%. In other embodiments, such as when being used as reference or research reagents, anti-NRP2a antibodies can be of lesser purity, and may have a purity of at least about 50%, 60%, 70%, or 80%. Purity can be measured overall or in relation to selected components, such as other proteins, e.g., purity on a protein basis.

Purified anti-NRP2a antibodies can also be characterized according to their biological characteristics. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more canonical or non-canonical biological activities can be measured according to cell-based assays, including those that utilize a cellular binding partner of a selected anti-NRP2a antibody, which is functionally coupled to a readout or indicator, such as a fluorescent or luminescent indicator of biological activity, as described herein.

In certain embodiments, as noted above, an anti-NRP2a antibody composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, an anti-NRP2a antibody composition is made from a eukaryotic cell such as a mammalian or human cell in substantially serum free media. In certain embodiments, as noted herein, an anti-NRP2a antibody composition has an endotoxin content of less than about 10 EU/mg of anti-NRP2a antibody, or less than about 5 EU/mg of anti-NRP2a antibody, less than about 3 EU/mg of anti-NRP2a antibody, or less than about 1 EU/mg of anti-NRP2a antibody.

In certain embodiments, an anti-NRP2a antibody composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see. e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isoelectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins such as protein fragments and antibodies can be obtained by utilizing a combination of chromatographic materials in at least two orthogonal steps, among other methods (see, e.g., Therapeutic Proteins; Methods and Protocols. Vol. 308. Eds., Smales and James, Humana Press Inc., 2005). Typically, protein agents (e.g., anti-NRP2a antibodies, and antigen-binding fragments) are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of anti-NRP2a antibodies or variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see. e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Anti-NRP2a antibodies with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains an anti-NRP2a antibody of the present disclosure.

Although the foregoing embodiments have been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Binding Affinities

The isoform specificity, binding affinities, and species reactivity for the antibodies listed in Tables A1/A2 were tested as described below to characterize their functional properties. Binding experiments were conducted by biolayer interferometry (BLI) on an Octet RED96e instrument (Sartorius). All antibodies, peptides, and proteins were diluted in 1×PBS, 0.1% BSA, and 0.02% Tween 20, pH 7.4. For isoform specificity and mouse reactivity, biotinylated peptides corresponding to the unique sequences of NRP2 isoforms a and b, and mouse NRP2 isoform a (see Table E2) were purchased from China Peptides and Genscript. The peptides were immobilized on Octet Streptavidin Biosensors (Sartorius, 18-5019) at 20 μg/mL. The biosensors were then dipped into 50 nM solutions of each antibody and read for 300s.

For measuring binding affinities, a biotinylated anti-mouse antibody (CaptureSelect biotin anti-LC-kappa murine, ThermoFisher) was immobilized on Octet Streptavidin Biosensors (Sartorius, 18-5019) at 8 μg/mL. The biosensors were then dipped into 1 μg/mL solutions of each antibody, followed by a titration of 450, 150, 50, 16.67, 5.56, 1.85, 0.62 nM recombinant NRP2 isoform a. The association phase of binding was 300s and the dissociation phase was 1200s. The biosensors were regenerated in 10 mM glycine, pH 1.5 in between cycles of different antibodies. Binding affinities were obtained by fitting the data to a 1:1 binding model in the Octet Data Analysis software

The results are summarized in Table E1 below.

TABLE E1
Antibody affinities, isoform specificity, and species reactivity

| | | Human | | |
|---|---|---|---|---|
| | | NRP2a | Human | |
| | NRP2 | binding | NRP2b | |
| | isoform | affinity | binding | Mouse |
| Antibody | specificity | (nM) | affinity | Reactivity |
| aNRP2-37v2 | a | 0.767 | None | Poor |
| aNRP2-400v2 | a | 0.453 | None | None |
| aNRP2-401v2 | a | 0.136 | None | Good |
| aNRP2-402v2 | a | 3.35 | None | Good |
| aNRP2-403v2 | a | 1.40 | None | Poor |
| aNRP2-404v2 | a | 0.552 | None | Poor |
| aNRP2-405v2 | a | 0.480 | None | Good |
| aNRP2-406v2 | a and b | 5.69 | Poor | Good |
| aNRP2-407v2 | a and b | 0.352 | Poor | Good |
| aNRP2-408v2 | a | 2.10 | None | Good |
| aNRP2-409v3 | a | 2.23 | None | Good |

These results demonstrate for the first time the creation of high affinity antibodies which show specific selectivity for NRP2a and little or no binding to NRP2b. Such antibodies demonstrate high (sub nanomolar) affinity for NRP2a with little or no nonspecific reactivity, demonstrating that the epitopes identified enable high specificity, and provide NRP2a antibodies with unique functional and diagnostic characteristics.

### Example 2

### Dimerization of NRP2 Isoforms and CCR7 Induced by CCL21

To determine the specificity of NRP2a and NRP2b association with CCR7, different protein isoforms of NRP2 were assessed for their ability to form heterodimers with CCR7 in the presence of chemokine ligands CCL21 and CCL19.

Briefly, vectors containing the small (pBiT2.1-C) and large(pBiT1.1-C) fragment of a split NanoLuc were obtained from Promega corporation. cDNA encoding human NRP2a (RCC220706, isoform 2) and human NRP2b (RC210928, isoform 5) were obtained from Origene Technologies. cDNA encoding CCR7 (OHu24012) was obtained from Genscript. N-terminal to the vector encoded spacer sequences and NanoLuc tags, the complete coding sequences of NRP2 were cloned into pBiT1.1-C, while the full coding sequence of CCR7 was cloned into pBiT1.1-C. The remaining protein isoforms of NRP2 were constructed from the previously described vectors following standard mutagenesis techniques.

The vectors were then transfected into Expi293F cells by Expifectamine transfection reagent (Fisher Scientific) at 1 ug per mL at a density of 1 million cells per mL. Approximately 16-20 hours post transfection, cells were washed, plated at 100,000 cells/well in luminometer plates, and then cell permeable luciferase substrate was added and luminescence was monitored on a GloMax96 (Promega) until stabilization of luminescent signal was achieved. 100 nM of either CCL 19 (R&D systems, 361-MI/CF) or CCL21 (R&D systems. 366-6C/CF) was added, and the change in luminescence of wells with added ligand compared to control wells was calculated.

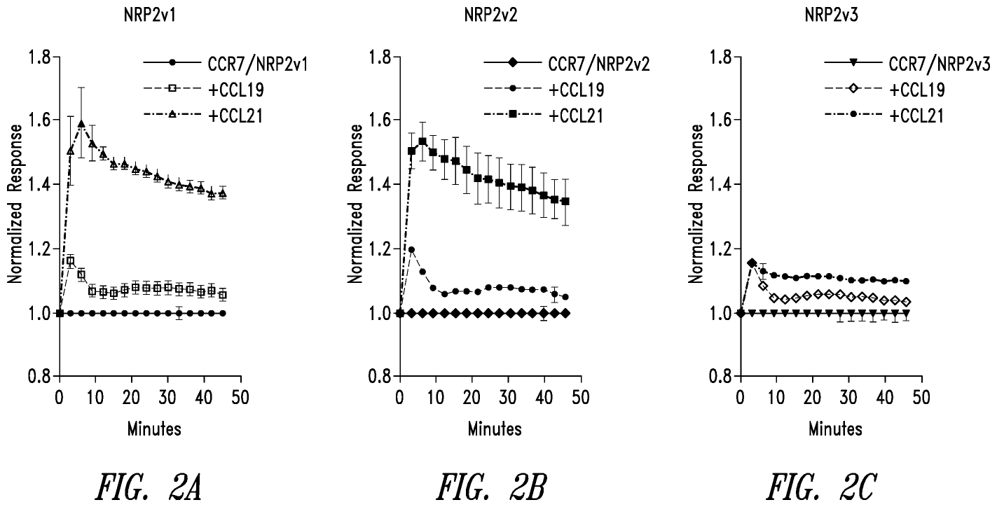

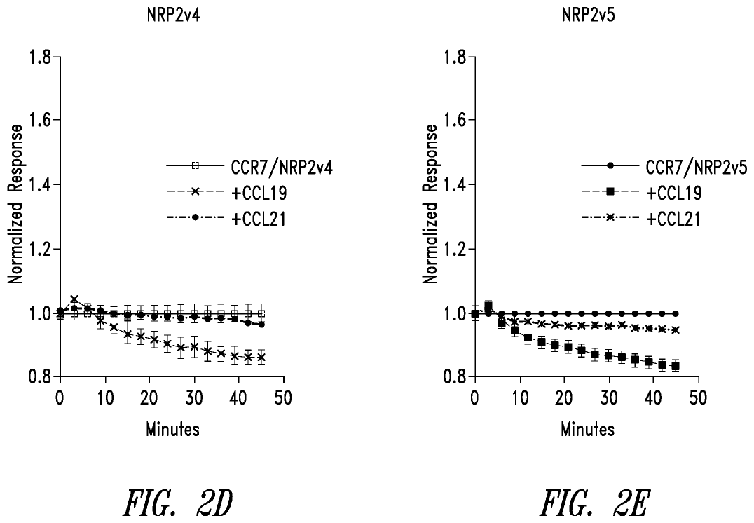

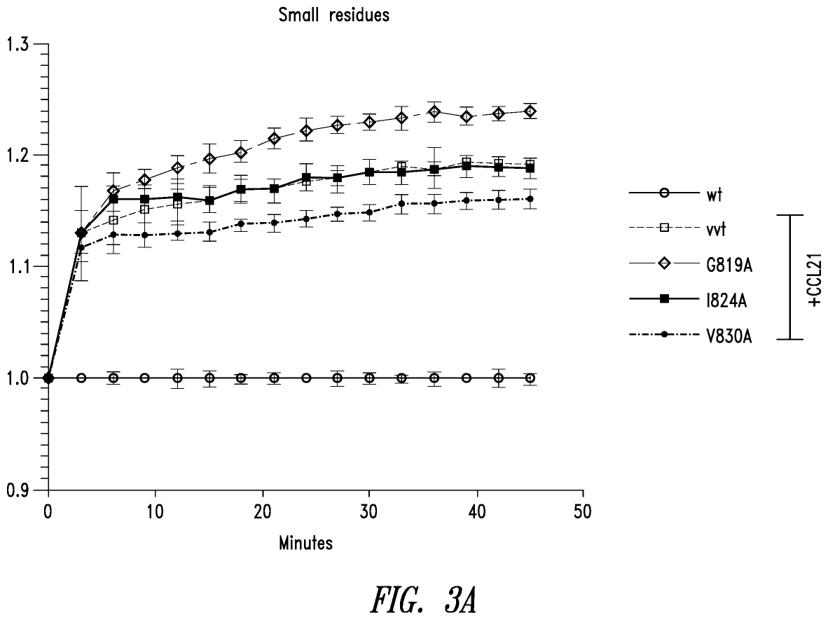

As shown in FIGS. 2A-2E, two isoforms of NRP2a. NRP2a v1 (931aa) and NRP2a v2 (926aa), showed enhanced association with CCR7 in the presence of CCL21 compared to CCL19, whereas both of the ‘b’ isoforms of NRP2b. NRP2b v4 (906aa) and NRP2b v5 (909aa), showed no response to chemokine ligands. This data suggests the presence of a CCL21 specific binding site in the juxtamembrane sequence of the NRP2a v1 and v2 isoforms but not the NRP2b isoforms. While all of the NRP2a variants (e.g., v1/v2/v3) are considered to be the ‘a’ isoforms of NRP2, NRP2v3 (909aa) showed no additional response to CCL21, suggesting the binding site is disrupted by the omission of 17aa from the juxtamembrane region in this variant.

This data is the first to demonstrate the specific interaction between NRP2a (but not NRP2b) and CCR7 in the presence of its ligand CCL21. Given the central role of CCR7/CCL21 axis in the regulation of immune cell migration activation, tissue inflammation, autoimmunity, and cancer progression, this data provides direct evidence that NRP2a-specific antibodies would have highly selective and specific effects on CCR7/CCL21 signaling, and the associated pathophysiology associated with these pathways.

### Example 3

### Mutagenesis of CCL21 Binding Site on NRP2a and Effects on Ligand-Induced Dimerization of NRP2a and CCR7

To identify residues responsible for the interaction of CCL21 with NRP2a, alanine scanning mutagenesis was performed through the juxtamembrane region of NRP2a expected to be disrupted in the NRP2v3 deletion. Then, receptor dimerization experiments were performed to identify the specific amino acids involved in creating the binding interaction.

Mutations were made in the NanoBiT vector pBiT2.1-C-NRP2v2 (isoform 2) following standard site directed mutagenesis protocols. The CCR7 protein in pBiT1.1-C in this case was truncated to remove the cytoplasmic sequence C terminal to the last (7^{th}) transmembrane helix (after aa331), to remove signal responses likely due to receptor trafficking which were seen with CCL 19 and CCL21 with NRP2v3.

The vectors were transfected into Expi293 cells using Expifectamine (Fisher Scientific). Approximately 16-20 hours post transfection, cells were washed, plated at 100,000 cells/well in luminometer plates, and then cell permeable luciferase substrate was added and luminescence was monitored on a GloMax96 (Promega). 100 nM ligand was added, and response to ligand was calculated with respect to a control set with no ligand.

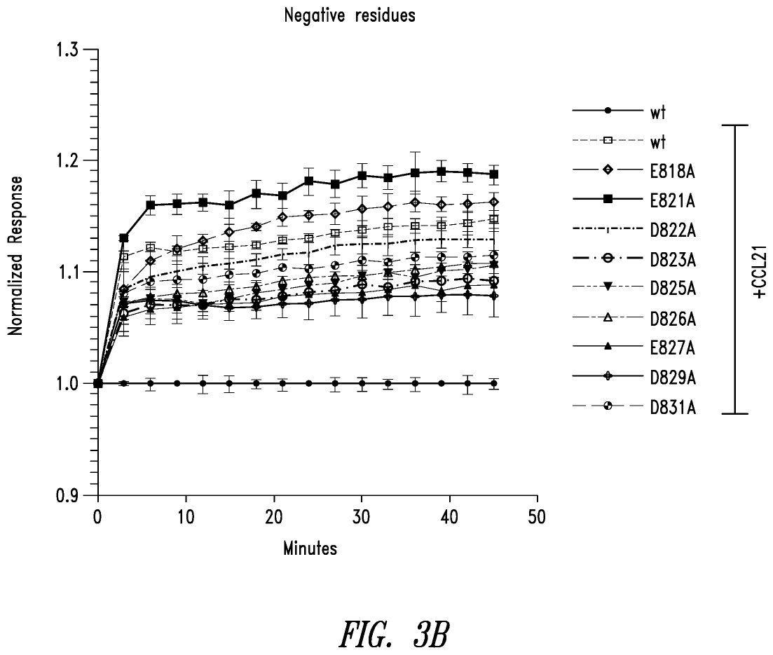

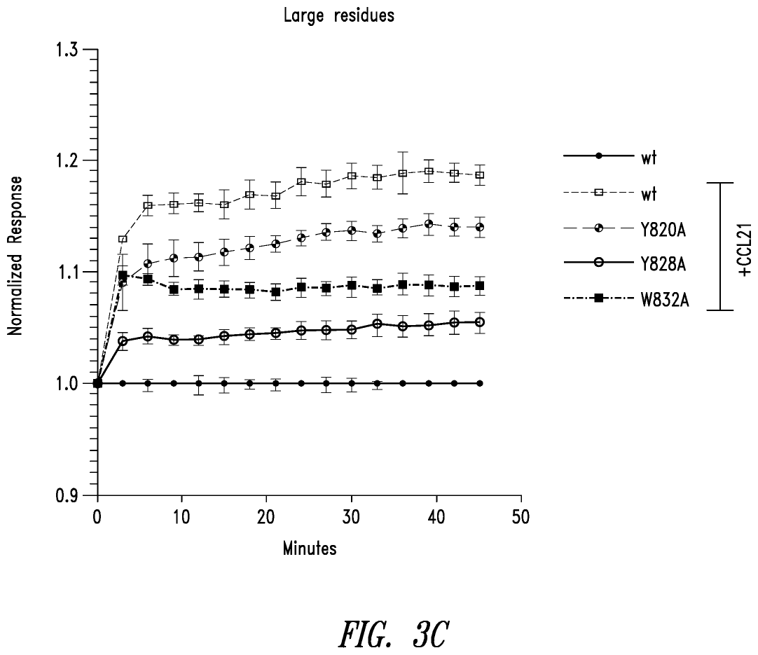

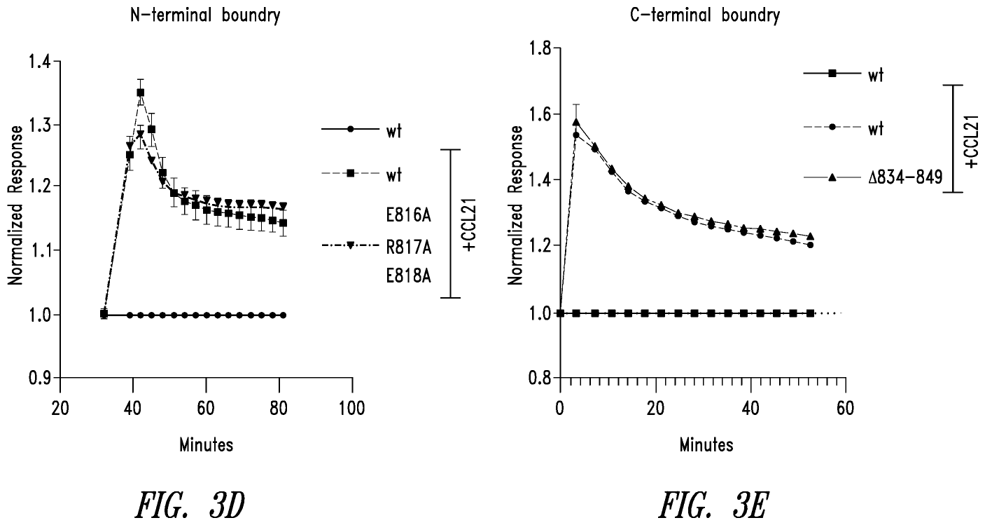

As shown in FIGS. 3A-3E, mutations showed various degrees of decreased binding with the substitution of negatively charged residues throughout the binding site, and highlighted the critical importance of a tyrosine residue at position 828 of NRP2a isoform v2. Additionally, a 3 amino acid substitution from residues 816-818, and a deletion from residues 834-849 showed nominal effects on receptor dimerization, in this case with the full CCR7 sequence, suggesting that these sequences are not important for receptor interaction. These regions define the N- and C-terminal boundaries to the binding site. Accordingly, residues 819-833 of FL NRP2a define the unique minimum epitope which is involved in the interaction of NRP2a with CCL21.

### Example 4

### ELISA Specificity of Antibodies to NRP2 Peptides

To determine the approximate epitopes recognized by each NRP2a reactive antibody, all antibodies were tested against NRP2 peptides generated by Genscript and China Peptides via immunoassay, as described below. All antibodies were also tested for human/mouse conservation and cross-reactivity with NRP2b. The NRP2 peptides used for mapping are shown in Table E2, and include a series of 12 amino acid peptide sequences which systematically span the minimum CCL21 interacting domain on NRP2a defined in Example 3. Of these peptide sequences, peptides NRP2a-scan 4, 5, and 6 represent the sequences which most completely cover the CCL21 interacting domain.

TABLE E2

| | | SEQ |
|---|---|---|
| | | ID |
| Peptide | Sequence | NO: |
| NPR2A | PISAFAVDIPEIHEREGYEDEIDDEYEVD | 113 |
| | WSNSSSATSGSGAPSTDKEKSWLYTLDP | |
| Short | DIPETHEREGYEDEIDDEYEVDW | 114 |
| NRP2A | | |
| NRP2a_scan | VDIPEIHEREGY | 115 |
| 1 | | |
| NRP2a_scan | PEIHEREGYEDE | 116 |
| 2 | | |
| NRP2a_scan | HEREGYEDEIDD | 117 |
| 3 | | |
| NRP2a_scan | EGYEDEIDDEYE | 118 |
| 4 | | |
| NRP2a_scan | EDEIDDEYEVDW | 119 |
| 5 | | |
| NRP2a_scan | IDDEYEVDWSNS | 120 |
| 6 | | |
| NRP2a_scan | EYEVDWSNSSSA | 121 |
| 7 | | |
| Mid-NRP2A | EGYEDEIDDEYEVDWSNSSSATS | 122 |
| Mid-mNRP2A | EGYEDEIDDEYEGDWSNSSSSTS | 123 |
| mNRP2A | PISAFAVDIPETHGGEGYEDEIDDEYEGD | 124 |
| | WSNSSSSTSGAGDPSSGKEKSWLYTLDP | |
| NRP2b | PISAFAGGTLLPGTEPTVDTVPMQPIPAYW | 125 |
| mNRP2b | PISAFAGGTLPPGTEPTVDTVPVOPIPAYW | 126 |

The peptides were conjugated to biotin. Streptavidin plates were coated with each peptide at 2 ug/mL, diluted in Casein. Plates were sealed with a plate sealer and were incubated at 4° C. overnight (no shaking). After overnight coating, the plates were washed three times with PBST. Each NRP2a specific antibody of interest was diluted to 1 ug/mL, 0.5 ug/mL, and 0.25 ug/mL and 50 uL/well was added to the assay plates. Plates were incubated at room temperature for 1 hour with shaking (400 rpm). Plates were washed again three times with PBST and HRP conjugated Goat-anti-mouse IgG (Jackson Immuno Research, 115-035-071) was added at a 1:5000 dilution at 50 uL/well. Plates were incubated at room temperature for 1 hour with shaking (400 rpm). Plates were washed three tines with PBST and were developed by adding 50 uL/well of 1-Step Ultra TMB substrate (Thermo Scientific, 34029).

Plates were incubated with substrate for 10 minutes and the reaction was stopped with Stop Solution (Biolegend, 423001). The colorimetric signal was read at 450 nm on a Biotek plate reader. If signals were <3× the average blank, the antibody was labeled as non-binding to the peptides. Signals that were >3× blank but <10× blank were labeled as positive but low signal. Signals that were >10× blank was a positive binder to a peptide. The results are shown in Table E3.

TABLE E3

| | 400 | 401 | 402 | 403 | 404 | 405 | 406 | 407 | 408 | 409 | 37 |
|---|---|---|---|---|---|---|---|---|---|---|---|
| Pept. | v2 | v2 | v2 | v2 | v2 | v2 | v2 | v2 | v2 | v2 | v2 |
| NRP2A | y | y | y | y | y | y | y | y | y | y | y |
| NRP2a_ | y | n | n | n | y | n | n | n | n | n | n |
| scan1 | | | | | | | | | | | |
| NRP2a_ | n | n | n | n | y | n | n | n | n | n | n |
| scan2 | | | | | | | | | | | |
| NRP2a_ | n | n | n | n | n | n | n | n | n | n | n |
| scan3 | | | | | | | | | | | |
| NRP2a_ | n | n | n | n | n | n | n | n | n | n | n |
| scan4 | | | | | | | | | | | |
| NRP2a_ | n | y | y | y | n | n | n | n | n | n | n |
| scan5 | | | | | | | | | | | |
| NRP2a_ | n | y | y | y | n | n | n | n | n | n | n |
| scan6 | | | | | | | | | | | |
| NRP2a_ | n | n | n | n | n | n | n | n | n | n | n |
| scan7 | | | | | | | | | | | |
| Mid- | n | y | low | y | n | n | n | n | n | n | n |
| NRP2A | | | | | | | | | | | |
| Mid- | n | y | low | y | n | n | n | n | n | n | n |
| mNRP2A | | | | | | | | | | | |
| mNRP2A | n | y | y | y | low | y | y | y | y | y | y |
| NRP2b | n | n | n | n | n | n | y | y | n | n | n |

The results demonstrate that antibodies aNRP2-406v2 and aNRP2-407v2 showed cross-reactivity with both NRP2a and NRP2b, but did not bind to peptides representing the minimum CCL21 interaction region. By comparison, antibodies aNRP2-401v2, aNRP2-402v2, and aNRP2-403v2 showed selectivity for NRP2a peptides scan 5 and 6, as well as the longer NRP2a juxtamembrane sequences, but did not bind to NRP2b. Antibodies aNRP2-400v2 and aNRP2-404v2 likely bind further N-terminal in the juxtamembrane region, but may also be of sufficient proximity to the binding site to block, while the remaining antibodies likely bind the C-terminus of the juxtamembrane region.

### Example 5

### Biolayer Interferometry Specificity of Antibodies to NRP2 Peptides

Biolayer interferometry (BLI) experiments were carried out on an Octet RED96e instrument (Sartorius) to further characterize antibody affinities to the NRP2 peptides screened above. Antibodies were epitope mapped using peptides that span the NRP2a isoform specific region; antibodies were also tested for reactivity to an NRP2b isoform specific peptide and cross-reactivity to mNRP2a and mNRP2b peptides (see Table E2 for peptide sequences).

Peptides were synthesized at Genscript and China Peptides and were biotin-conjugated. Antibodies and peptides were diluted in 1×PBS, 0.1% BSA, 0.02% Tween 20, pH 7.4. The peptides were immobilized on Octet Streptavidin Biosensors (Sartorius, 18-5019) at 20 μg/mL. The biosensors were then dipped into 50 nM solutions of each antibody and read for 300s. The biosensors were regenerated in 10 mM glycine, pH 1.5 in between cycles of different antibodies. The results are shown in Table E4, and confirm the data presented above.

TABLE E4

| | 400 | 401 | 402 | 403 | 404 | 405 | 406 | 407 | 408 | 409 | 37 |
|---|---|---|---|---|---|---|---|---|---|---|---|
| Pept. | v2 | v2 | v2 | v2 | v2 | v2 | v2 | v2 | v2 | v2 | v2 |
| NRP2A | y | y | y | y | y | y | y | y | y | y | y |
| NRP2a_ | y | n | n | n | y | n | n | n | n | n | n |
| scan1 | | | | | | | | | | | |
| NRP2a_ | n | n | n | n | y | n | n | n | n | n | n |
| scan2 | | | | | | | | | | | |
| NRP2a_ | n | n | n | n | n | n | n | n | n | n | n |
| scan3 | | | | | | | | | | | |
| NRP2a_ | n | n | n | n | n | n | n | n | n | n | n |
| scan4 | | | | | | | | | | | |
| NRP2a_ | n | y | y | y | n | n | n | n | n | n | n |
| scan5 | | | | | | | | | | | |
| NRP2a_ | n | y | y | y | n | n | n | n | n | n | n |
| scan6 | | | | | | | | | | | |
| NRP2a_ | n | n | n | n | n | n | n | n | n | n | n |
| scan7 | | | | | | | | | | | |
| Mid- | n | y | y | y | n | n | n | n | n | n | n |
| NRP2A | | | | | | | | | | | |
| Short- | y | n | n | n | y | n | n | n | n | n | n |
| NRP2A | | | | | | | | | | | |
| Mid- | n | y | y | y | n | n | n | n | n | n | n |
| mNRP2A | | | | | | | | | | | |
| mNRP2A | n | y | y | y | y | y | y | y | y | y | y |
| NRP2b | n | n | n | n | n | n | y | y | n | n | n |
| mNRP2b | n | n | n | n | n | n | y | y | n | n | n |

The results confirm that antibodies aNRP2-406v2 and aNRP2-407v2 showed cross-reactivity with both NRP2a and NRP2b, but failed to bind peptides representing the minimum CCL21 interaction region, and that antibodies aNRP2-401v2, aNRP2-402v2, and aNRP2-403v2 showed selectivity for the minimum epitope that interacts with CCL21 on NRP2a. These results also confirmed that the antibodies aNRP2-404v2, aNRP2-405v2, aNRP2-408v2, aNRP2-409v2, and aNRP2-37v2 show strong specificity for NRP2a, even though they do not bind to the CCL21 interacting minimum domain.

### Example 6

### Cell Surface Recognition of NRP2 by Antibodies

Selected NRP2a reactive antibodies were evaluated for their ability to recognize human or mouse NRP2a receptor, as well as for their specificity to NRP2a over NRP2b, cDNAs encoding human NRP2a v2 (RCC220706), mouse NRP2a (MR224748) or human NRP2b (RC210928) were obtained from Origene technologies. An NRP2 v3 expression vector was then constructed by modifying the vector encoding NRP2 v2 via mutagenesis to remove the 17 amino acids missing in NRP2 v3. Expi293F cells were transfected with each vector and ˜48 hours post transfection counted and stained with each recombinant antibody and fluorescent secondary (Jackson Immuno research, 115-175-146). Signal to noise was calculated as the staining intensity of transfected cells compared to an un-transfected ‘mock’ population. Binding was compared to an NRP2 mouse/human reactive al domain binding antibody (aNRP2-17v2) and a non-binding isotype control (NBIC).

The results are shown in FIGS. 4A-4C. The tested antibodies showed varied binding to cells expressing full length NRP2a or NRP2b. Based on these studies, antibody aNRP2-403 showed the best binding to human NRP2a expressed recombinantly on Expi293F cells. Antibody aNRP2-403v2 also showed strong binding to NRP2a, and additionally has the advantage of showing some mouse cross reactivity. Antibody aNRP2-402 showed good binding specificity for NRP2a, but also exhibited lower affinity compared to the other antibodies tested. Based on these studies, antibody aNRP2-403v2 may possess the ability to bind to other regions of NRP2 beyond its NRP2a peptide-specific binding, as it is non-reactive to NRP2b specific peptides, but appears to show some binding to full length NRP2b expressed recombinantly in living cells in this model.

Additionally, of the antibodies shown to bind the CCL21 minimum interacting domain, aNRP2-401 shows good binding, preservation of mNRP2a binding, and good specificity for NRP2a. Cell surface binding of NRP2 v2 compared to NRP2 v3 shows that while the missing amino acids are sufficient to prevent CCL21 associated NRP2v3/CCR7 association (see Example 2), antibodies targeting near this region may not be specific for NRP2 v2 over NRP2 v3 as the entirety of the binding site is not removed.

### Example 7

### Antibody Competition of NRP2a/CCR7 Receptor Dimerization

To confirm the ability of antibodies to block NRP2a/CCR7-CCL21 induced receptor dimerization, antibody blocking experiments were performed, pBiT1.1-C—CCR7 (without its C-terminal cytoplasmic sequence) and pBiT2.1-C-NRP2v2 (isoform 2) were co-transfected in Expi293 cells with Expifectamine transfection reagent. 16-20 hours post transfection cells were counted and plated and luciferase substrate was added. Each indicated antibody was added at 10 nM, and a new baseline luminescence signal was established, then CCL21 was added at EC80, and normalized response to ligand was observed over time. The area under the curve was then computed and compared.

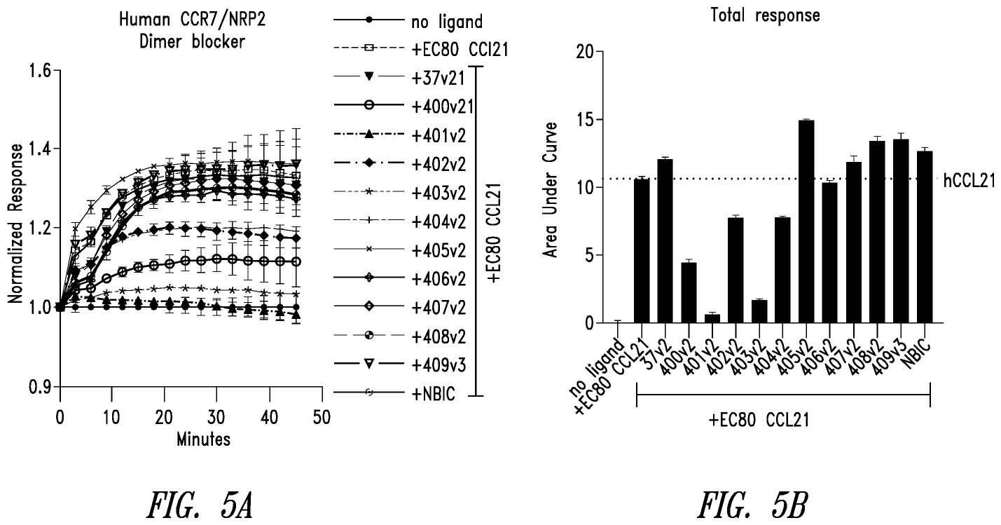

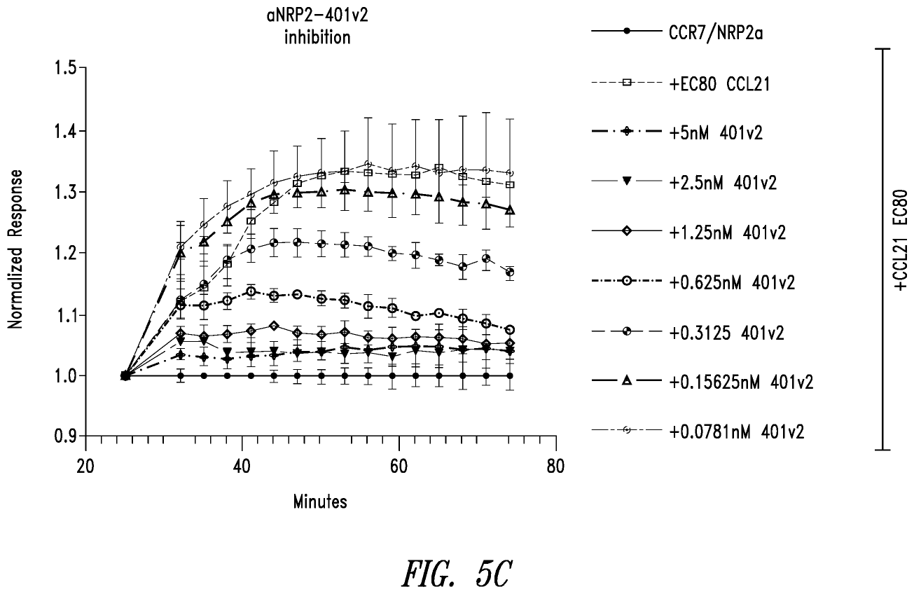

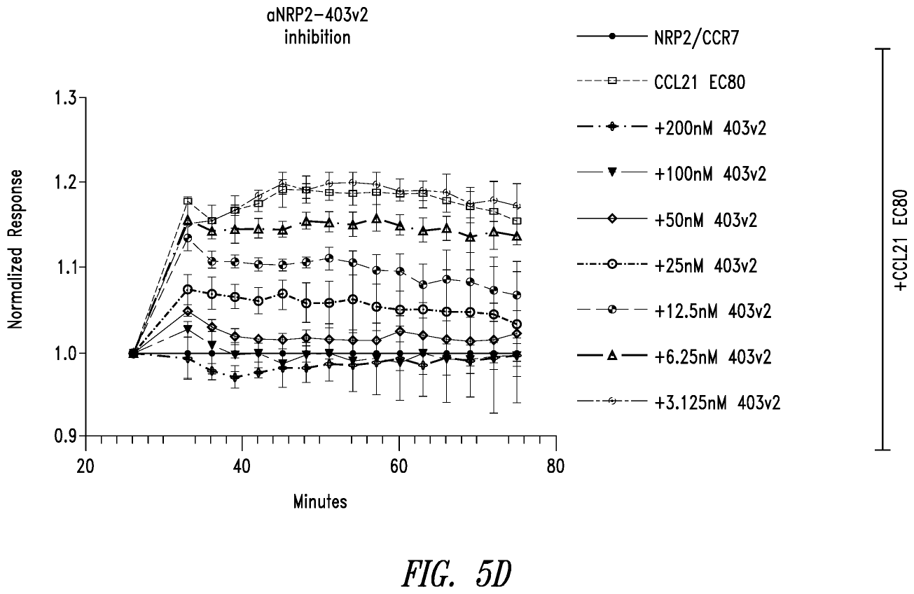

The results are shown in FIGS. 5A-5D. Antibodies aNRP2-400v2, aNRP2-401v2, aNRP2-402v2, aNRP2-403v2, and aNRP2-404v2 all showed some degree of blocking, with antibodies aNRP2-401v2 and aNRP2-403v2 being the most potent. Other antibodies appeared to have negligible effect on receptor dimerization, similar to that seen with a non-binding isotype control (NBIC). Titrations of antibodies aNRP2-401v2 and aNRP2-403v2 against EC80 of ligand gave respective IC50s of 0.339 nM and 12.72 nM. This data confirms that antibodies aNRP2-401v2 and aNRP2-403v2 have utility in therapeutic approaches aimed at disrupting the interaction of NRP2a and CCL21.

### Example 8

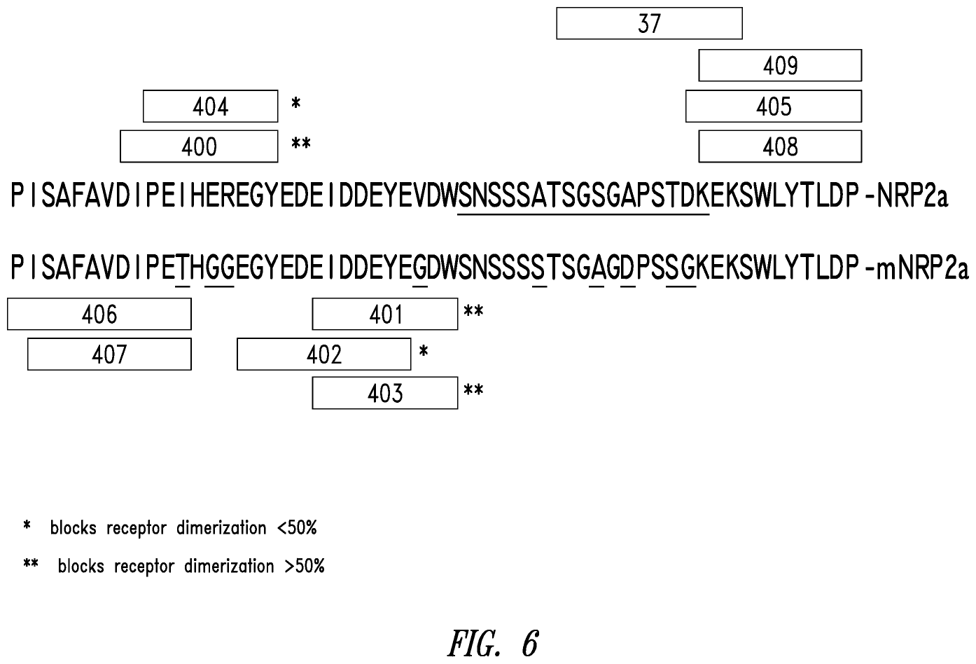

Epitope mapping of antibodies to presumed binding site of CCL21 Mapping based on peptide binding boundaries and human/mouse conservation of NRP2a was performed with the selected antibodies shown in FIG. 6; here, substitutions relative to human sequence in mNRP2a are underlined in the mouse sequence (mNRP2a), and the CCL21 binding site is shown in bold in the human sequence. Underlined in the human sequence is the deletion described in Example 3 (FIG. 3E) that was ineffective at preventing CCL21 induced dimerization, and thus serves as an approximate C-terminal boundary of the binding site. The N-terminal boundary is not labelled, but is evidenced from the described mutations in NRP2 v2 (816-818) in Example 3 (FIG. 3D) that showed minimal effects on CCL21 induced dimerization.

Antibodies with epitopes which overlap the binding site of CCL21 (i.e., aNRP2-401v2, aNRP2-402v2, and aNRP2-403v2) are efficient blockers of receptor dimerization with CCR7. Similar to above, these antibodies demonstrate utility in therapeutic approaches aimed at disrupting the interaction of NRP2a and CCL21

Also, binding to the NRP2a specific peptide itself (for example, as illustrated by antibodies aNRP2-400v2, aNRP2-404v2, aNRP2-405v2, aNRP2-408v2, aNRP2-409v2, and aNRP2-37v2) is not sufficient to block receptor dimerization with high efficiency. These antibodies show utility as diagnostic reagents, for example, as reagents to develop target engagement, and measure receptor density on cells and in tissues.

### Example 9

### Humanized Antibodies

Humanized antibodies were prepared from antibodies aNRP2-401v2 and aNRP2-402v2, the CDRs were identified by their Kabat definition (with the exception of CDR-H1 which uses the IMGT boundaries) as defined in Table A1. The full V-genes of the mouse monoclonal light and heavy chains were aligned against the human V-genes to identify the most similar framework sequences, and a suitable human framework was identified for transfer. Mouse CDRs were transferred onto human frameworks and affinities were determined by binding to a range of NRP2 ligand concentrations by bio-layer interferometry (BLI) on an Octet RED96e. Where necessary back-mutations were made in the human frameworks taking into account residues important for the V_{L}V_{H} interface, CDR loop contacts, and other structural considerations. Binding was again measured and critical back mutations were selected. Additionally, antibodies were affinity matured via NNK saturation mutagenesis through CDRs to improve affinity to ligand.

The binding affinities of the humanized variants are shown in Table E5 below.

TABLE E5
Humanized variant affinity to NRP2a

| Sample ID | KD (M) | ka (1/Ms) | kdis (1/s) |
|---|---|---|---|
| aNRP2-401v5 | 3.91E−10 | 7.48E+05 | 2.93E−04 |
| aNRP2-401v6 | 1.62E−10 | 9.99E+05 | 1.62E−04 |
| aNRP2-401v7 | 1.41E−10 | 1.23E+06 | 1.73E−04 |
| aNRP2-401v8 | 9.89E−11 | 1.21E+06 | 1.20E−04 |

### Example 10

### Variants of aNRP2-401

Antibody variants of humanized aNRP2-401 were generated by site-directed mutagenesis through standard protocols. The entirety of the light chain CDR3, the heavy chain CDR3, and the first 11 amino acids of heavy chain CDR2 were randomly mutated. For each position, 70-100 individual clones were screened, yielding 15-16 amino acid variants for each after omitting Cysteine. Tryptophan, and Methionine. Variants were then transfected into Expi293 cells (ThermoFisher, A14527) as single substitutions along with an unmodified cognate light or heavy chain. The concentration of each variant in the solution was then determined on an Octet RED96e, and the variants were then used to compete with the control antibody in a competition ELISA.

Humanized aNRP2-401 was biotinylated using EZ-link NSH-PEG₄-Biotin (ThermoFisher. 21363) with a 20-fold molar excess of biotin to IgG. Via an indirect ELISA, a biotinylated antibody was titrated against NRP2 (amino acids 23-855 of transcript variant 2) coated at 2 ug/mL in 1×PBS pH 7.4 to determine an appropriate EC₅₀. The competing antibody was diluted by 3 or 2-fold dilutions with the biotinylated control antibody at EC₅₀. All plates were blocked with Casein Blocker (ThermoFisher, 37528), washed with PBST pH 7.4, detected with Streptavidin-HRP (ThermoFisher, SA 10001), and developed with TMB Ultra (ThermoFisher, 37574). Data analysis was performed using GraphPad Prism.

IC₅₀ values for each variant were calculated from the competition curves. Variants that expressed, and were judged to sufficiently retain binding to antigen, had an IC₅₀ no greater than 2-fold of the control and similar hill slopes. The achieve binding variants are summarized in Table E6 and Table E7, which show their relative position within the CDRs and each amino acid variant that retained binding.

TABLE E6
Antibody aNRP2-401 CDR Consensus
Sequences from Variant Testing

| | | SEQ ID |
|---|---|---|
| Chain | Sequence | NO: |
| V_{H}CDR1 | GFTFSDYGMH | 13 |
| V_{H}CDR2 | X₁₄X₁₅X₁₆X₁₇X₁₈X₁₉X₂₀ | 127 |
| | X₂₁X₂₂X₂₃X₂₄ADTVKG | |
| V_{H}CDR3 | GX₁X₂X₃X₄X₅ | |
| V_{L}CDR1 | RSSQSLVHSNGNTYLY | 16 |
| V_{L}CDR2 | KVSNRFS | 17 |
| V_{L}CDR3 | X₆X₇X₈X₉X₁₀X₁₁X₁₂X₁₃ | |

TABLE E7
Amino acid definitions for CDR Consensus Sequences

| | |
|---|---|
| X₁ | G, A, or S |
| X₂ | Y, E, K, L, or R |
| X₃ | T, A, G, I, L, Q, or V |
| X₄ | D, A, G, K, N, Q, R, or S |
| X₅ | Y, A, D, E, F, G, H, I, K, L, N, Q, R, S, T, or V |
| X₆ | S, A, G, I, L, P, T, or V |
| X₇ | Q, A, G, R, or S |
| X₈ | S, A, H, K, L, Q, or T |
| X₉ | T, F, G, H, I, K, L, N, Q, R, S, V, or Y |
| X₁₀ | H, A, D, E, F, G, I, K, L, N, Q, R, S, T, or Y |
| X₁₁ | V, A, E, F, G, H, I, K, L, N, P, Q, R, S, T, or Y |
| X₁₂ | L, A, E, H, I, N, P, Q, S, T, or V |
| X₁₃ | T, A, D, E, F, G, I, K, L, N, Q, R, S, or V |
| X₁₄ | T, A, F, I, K, L, Q, R, or S |
| X₁₅ | I, A, F, G, L, N, Q, R, S, T, V, or Y |
| X₁₆ | S, A, F, G, H, I, K, L, P, Q, R, V, or Y |
| X₁₇ | R, A, G, K, L, N, P, Q, S, T, V, or Y |
| X₁₈ | D, A, G, Q, R, S, V, or Y |
| X₁₉ | I, A, E, G, H, K, L, N, P, R, S, T, or V |
| X₂₀ | N, A, D, I, L, P, Q, R, S, T, V, or Y |
| X₂₁ | T, A, G, H, L, N, P, R, S, or V |
| X₂₂ | V, F, G, I, L, N, P, Q, S, T, or Y |
| X₂₃ | Y, F, H, I, K, N, or R |
| X₂₄ | Y, A, D, F, G, H, I, K, L, N, P, Q, R, S, T, or V |

### Example 11

### In Vivo Model of Dendritic Cell Migration

An in-vivo model of dendritic cell migration was developed to test the role of NRP2 in dendritic cell migration from the skin to draining lymph nodes. On Day 0, 10 μl of a 1:1 ratio of Complete Freund's Adjuvant (CFA) (Sigma-Aldrich): phosphate-buffered saline (PBS) was injected subcutaneously into each ear of wild type and NRP2 knock-out mice with a 50 μl Hamilton syringe and a 22-gauge needle (Becton Dickinson). After 48 hours, each ear was painted on each side with 20 ul of Dibutyl phthalate with acetone (DPA) (Sigma-Aldrich) containing 5 mg/ml fluorescein isothiocyanate (FITC) (Fisher Scientific).

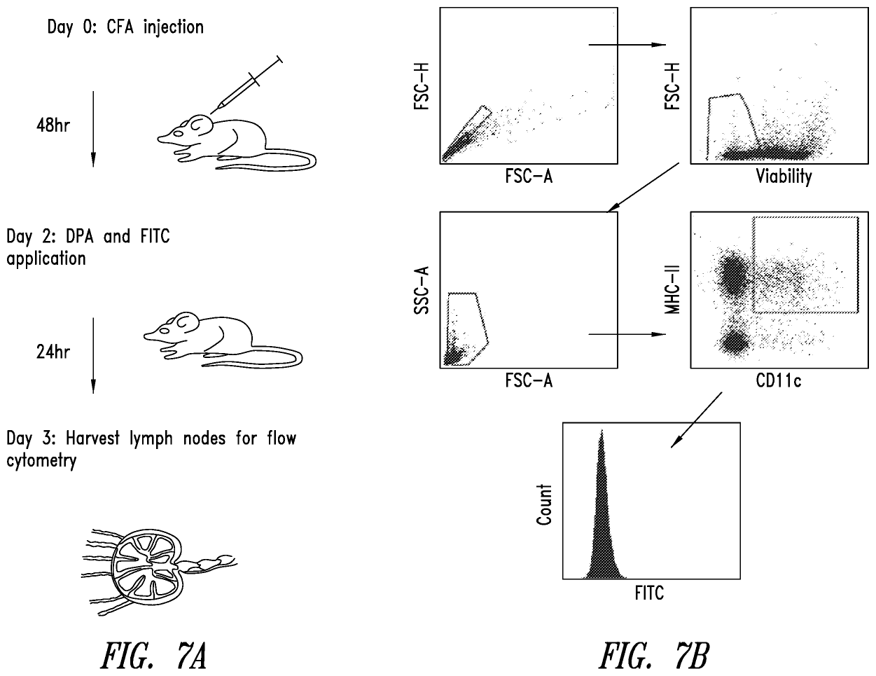

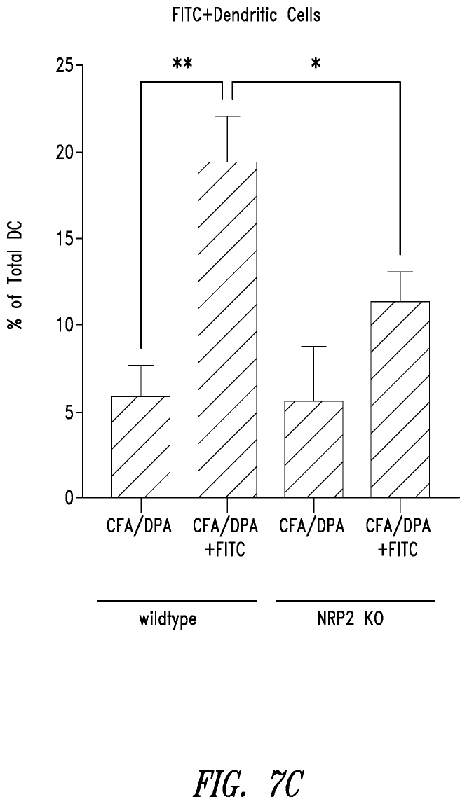

24 hours after DPA/FITC painting, mice were euthanized, draining lymph nodes removed, and processed for flow cytometric analysis (FIG. 7A). Briefly, single-cell suspensions were made by cutting lymph nodes and passing through a 70 um strainer (Fisher Scientific). Cells were stained with Zombie Aqua viability dye (Biolegend) prior to surface marker staining with anti-mouse CD3, CD11c, and MHC-II antibodies (Biolegend). Cells were then analyzed on a CytoFlex S (Becton Dickinson) flow cytometer to determine cell surface marker expression. FlowJo software was used to determine the proportion of FITC-positive dendritic cells (CD3⁻CD11c⁺MHC-II⁺) (FIG. 7B). As shown in FIG. 7C, a significant decrease of about 42% in the proportion of FITC⁺ dendritic cells was observed in the draining lymph nodes of NRP2 knockout mice compared to NRP2 wild-type mice, suggesting that NRP2 plays a role in the migration of dendritic cells in vivo. The same model is used to test the activity of the anti-NRP2 antibodies described herein, which are likewise expected to decrease dendritic cell migration from the skin to draining lymph nodes.

1. An antibody, or an antigen-binding fragment thereof, which binds to a neuropilin-2A (NRP2a) variant 1 (v1) or variant 2 (v2) polypeptide at an epitope that comprises, consists, or consists essentially of a sequence selected from Table N2, including about or at least about 8, 9, 10, 11, or 12 or more contiguous amino acids of a sequence selected from Table N2.

2. The antibody, or antigen-binding fragment thereof, of claim 1, wherein the epitope comprises, consists, or consists essentially of a sequence selected from SEQ ID NO: 96-104, including about or at least about 8, 9, 10, 11, or 12 contiguous amino acids of a sequence selected from SEQ ID NOs: 96-104.

3. The antibody, or antigen-binding fragment thereof, of claim 1 or 2, wherein the epitope comprises, consists, or consists essentially of SEQ ID NO: 100, or about or at least about 8, 9, 10, 11, or 12 contiguous amino acids of SEQ ID NO: 100.

4. The antibody, or antigen-binding fragment thereof, of any one of claims 1-3, comprising a heavy chain variable region (VH) sequence that comprises complementary determining region VHCDR1, VHCDR2, and VHCDR3 sequences, and a light chain variable region (VL) sequence that comprises complementary determining region VLCDR1, VLCDR2, and VLCDR3 sequences, wherein:
the VHCDR1, VHCDR2, and VHCDR3 sequences comprise SEQ ID NOs: 13, 127, and GX₁X₂X₃X₄X₅ (wherein X₁ is G, A, or S, X₂ is Y, F, K, L, or R, X₂ is T, A, G, I, L, Q, or V, X₄ is D, A, G, K, N, Q, R, or S, and X₅ is Y, A, D, E, F, G, H, I, K, L, N, Q, R, S, T, or V), respectively, and the VLCDR1, VLCDR2, and VLCDR3 sequences comprise SEQ ID NOs: 16, 17, and X₆X₇X₈X₉X₁₀X₁₁X₁₂X₁₃ (wherein X₆ is S, A, G, I, L, P, T, or V, X₇ is Q, A, G, R, or S, X₈ is S, A, H, K, L, Q, or T, X₉ is T, F, G, H, I, K, L, N, Q, R, S, V, or Y, X₁₀ is H, A, D, E, F, G, I, K, L, N, Q, R, S, T, or Y, X₁₁ is V, A, E, F, G, H, I, K, L, N, P, Q, R, S, T, or Y, X₁₂ is L, A, E, H, I, N, P, Q, S, T, or V, and X₁₃ is T, A, D, E, F, G, I, K, L, N, Q, R, S, or V), respectively (see also Table E6 and Table E7);
the VHCDR1, VHCDR2, and VHCDR3 sequences comprise SEQ ID NOs: 130-132, respectively, and the VLCDR1, VLCDR2, and VLCDR3 sequences comprise SEQ ID NOs: 133-135, respectively, including variants thereof having 1, 2, 3, 4, 5, or 6 total alterations across all of the CDR regions;
the VHCDR1, VHCDR2, and VHCDR3 sequences comprise SEQ ID NOs: 136-138, respectively, and the VLCDR1, VLCDR2, and VLCDR3 sequences comprise SEQ ID NOs: 139-141, respectively, including variants thereof having 1, 2, 3, 4, 5, or 6 total alterations across all of the CDR regions;
the VHCDR1, VHCDR2, and VHCDR3 sequences comprise SEQ ID NOs: 142-144, respectively, and the VLCDR1, VLCDR2, and VLCDR3 sequences comprise SEQ ID NOs: 145-147, respectively, including variants thereof having 1, 2, 3, 4, 5, or 6 total alterations across all of the CDR regions;
the VHCDR1, VHCDR2, and VHCDR3 sequences comprise SEQ ID NOs: 148-150, respectively, and the VLCDR1, VLCDR2, and VLCDR3 sequences comprise SEQ ID NOs: 151-153, respectively, including variants thereof having 1, 2, 3, 4, 5, or 6 total alterations across all of the CDR regions;
the VHCDR1, VHCDR2, and VHCDR3 sequences comprise SEQ ID NOs: 154-156, respectively, and the VLCDR1, VLCDR2, and VLCDR3 sequences comprise SEQ ID NOs: 157-159, respectively, including variants thereof having 1, 2, 3, 4, 5, or 6 total alterations across all of the CDR regions;
the VHCDR1, VHCDR2, and VHCDR3 sequences comprise SEQ ID NOs: 1-3, respectively, and the VLCDR1, VLCDR2, and VLCDR3 sequences comprise SEQ ID NOs: 4-6, respectively, including variants thereof having 1, 2, 3, 4, 5, or 6 total alterations across all of the CDR regions;
the VHCDR1, VHCDR2, and VHCDR3 sequences comprise SEQ ID NOs: 7-9, respectively, and the VLCDR1, VLCDR2, and VLCDR3 sequences comprise SEQ ID NOs: 10-12, respectively, including variants thereof having 1, 2, 3, 4, 5, or 6 total alterations across all of the CDR regions;
the VHCDR1, VHCDR2, and VHCDR3 sequences comprise SEQ ID NOs: 13-15 respectively, and the VLCDR1, VLCDR2, and VLCDR3 sequences comprise SEQ ID NOs: 16-18, respectively, including variants thereof having 1, 2, 3, 4, 5, or 6 total alterations across all of the CDR regions;
the VHCDR1, VHCDR2, and VHCDR3 sequences comprise SEQ ID NOs: 19-21, respectively, and the VLCDR1, VLCDR2, and VLCDR3 sequences comprise SEQ ID NOs: 22-24, respectively, including variants thereof having 1, 2, 3, 4, 5, or 6 total alterations across all of the CDR regions;
the VHCDR1, VHCDR2, and VHCDR3 sequences comprise SEQ ID NOs: 25-27, respectively, and the VLCDR1, VLCDR2, and VLCDR3 sequences comprise SEQ ID NOs: 28-30, respectively, including variants thereof having 1, 2, 3, 4, 5, or 6 total alterations across all of the CDR regions;
the VHCDR1, VHCDR2, and VHCDR3 sequences comprise SEQ ID NOs: 31-33, respectively, and the VLCDR1, VLCDR2, and VLCDR3 sequences comprise SEQ ID NOs: 34-36, respectively, including variants thereof having 1, 2, 3, 4, 5, or 6 total alterations across all of the CDR regions;
the VHCDR1, VHCDR2, and VHCDR3 sequences comprise SEQ ID NOs: 37-39, respectively, and the VLCDR1, VLCDR2, and VLCDR3 sequences comprise SEQ ID NOs: 40-42, respectively, including variants thereof having 1, 2, 3, 4, 5, or 6 total alterations across all of the CDR regions;
the VHCDR1, VHCDR2, and VHCDR3 sequences comprise SEQ ID NOs: 43-45, respectively, and the VLCDR1, VLCDR2, and VLCDR3 sequences comprise SEQ ID NOs: 46-48, respectively, including variants thereof having 1, 2, 3, 4, 5, or 6 total alterations across all of the CDR regions;
the VHCDR1, VHCDR2, and VHCDR3 sequences comprise SEQ ID NOs: 49-51, respectively, and the VLCDR1, VLCDR2, and VLCDR3 sequences comprise SEQ ID NOs: 52-54, respectively, including variants thereof having 1, 2, 3, 4, 5, or 6 total alterations across all of the CDR regions;
the VHCDR1, VHCDR2, and VHCDR3 sequences comprise SEQ ID NOs: 55-57, respectively, and the VLCDR1, VLCDR2, and VLCDR3 sequences comprise SEQ ID NOs: 58-60, respectively, including variants thereof having 1, 2, 3, 4, 5, or 6 total alterations across all of the CDR regions; or
the VHCDR1, VHCDR2, and VHCDR3 sequences comprise SEQ ID NOs: 61-63, respectively, and the VLCDR1, VLCDR2, and VLCDR3 sequences comprise SEQ ID NOs: 64-66, respectively, including variants thereof having 1, 2, 3, 4, 5, or 6 total alterations across all of the CDR regions.

5. The antibody, or antigen-binding fragment thereof, of claim 4, wherein:
the V_{H} sequence comprises SEQ ID NO: 170, and the V_{L} sequence comprises SEQ ID NO: 171;
the V_{H} sequence is at least 80, 85, 90, 95, 97, 98, 99, or 100% identical to SEQ ID NO: 160, and the V_{L} sequence is at least 80, 85, 90, 95, 97, 98, 99, or 100% identical to SEQ ID NO: 161;
the V_{H} sequence is at least 80, 85, 90, 95, 97, 98, 99, or 100% identical to SEQ ID NO: 162, and the V_{L} sequence is at least 80, 85, 90, 95, 97, 98, 99, or 100% identical to SEQ ID NO: 163;
the V_{H} sequence is at least 80, 85, 90, 95, 97, 98, 99, or 100% identical to SEQ ID NO: 164, and the V_{L} sequence is at least 80, 85, 90, 95, 97, 98, 99, or 100% identical to SEQ ID NO: 165;
the V_{H} sequence is at least 80, 85, 90, 95, 97, 98, 99, or 100% identical to SEQ ID NO: 166, and the V_{L} sequence is at least 80, 85, 90, 95, 97, 98, 99, or 100% identical to SEQ ID NO: 167;
the V_{H} sequence is at least 80, 85, 90, 95, 97, 98, 99, or 100% identical to SEQ ID NO: 168, and the V_{L} sequence is at least 80, 85, 90, 95, 97, 98, 99, or 100% identical to SEQ ID NO: 169;
the V_{H} sequence is at least 80, 85, 90, 95, 97, 98, 99, or 100% identical to SEQ ID NO: 67, and the V_{L} sequence is at least 80, 85, 90, 95, 97, 98, 99, or 100% identical to SEQ ID NO: 68;
the V_{H} sequence is at least 80, 85, 90, 95, 97, 98, 99, or 100% identical to SEQ ID NO: 69, and the V_{L} sequence is at least 80, 85, 90, 95, 97, 98, 99, or 100% identical to SEQ ID NO: 70;
the V_{H} sequence is at least 80, 85, 90, 95, 97, 98, 99, or 100% identical to SEQ ID NO: 71, and the V_{L} sequence is at least 80, 85, 90, 95, 97, 98, 99, or 100% identical to SEQ ID NO: 72;
the V_{H} sequence is at least 80, 85, 90, 95, 97, 98, 99, or 100% identical to SEQ ID NO: 73, and the V_{L} sequence is at least 80, 85, 90, 95, 97, 98, 99, or 100% identical to SEQ ID NO: 74;
the V_{H} sequence is at least 80, 85, 90, 95, 97, 98, 99, or 100% identical to SEQ ID NO: 75, and the V_{L} sequence is at least 80, 85, 90, 95, 97, 98, 99, or 100% identical to SEQ ID NO: 76;
the V_{H} sequence is at least 80, 85, 90, 95, 97, 98, 99, or 100% identical to SEQ ID NO: 77, and the V_{L} sequence is at least 80, 85, 90, 95, 97, 98, 99, or 1(00% identical to SEQ ID NO: 78;
the V_{H} sequence is at least 80, 85, 90, 95, 97, 98, 99, or 100% identical to SEQ ID NO: 79, and the V_{L} sequence is at least 80, 85, 90, 95, 97, 98, 99, or 100% identical to SEQ ID NO: 80;
the V_{H} sequence is at least 80, 85, 90, 95, 97, 98, 99, or 100% identical to SEQ ID NO: 81, and the V_{L} sequence is at least 80, 85, 90, 95, 97, 98, 99, or 100% identical to SEQ ID NO: 82;
the V_{H} sequence is at least 80, 85, 90, 95, 97, 98, 99, or 100% identical to SEQ ID NO: 83, and the V_{L} sequence is at least 80, 85, 90, 95, 97, 98, 99, or 100% identical to SEQ ID NO: 84;
the V_{H} sequence is at least 80, 85, 90, 95, 97, 98, 99, or 100% identical to SEQ ID NO: 85, and the V_{L} sequence is at least 80, 85, 90, 95, 97, 98, 99, or 100% identical to SEQ ID NO: 86; or
the V_{H} sequence is at least 80, 85, 90, 95, 97, 98, 99, or 100% identical to SEQ ID NO: 87, and the V_{L} sequence is at least 80, 85.90, 95, 97, 98, 99, or 100% identical to SEQ ID NO: 88.

6. The antibody, or antigen-binding fragment thereof, of any one of claims 1-5, which does not substantially bind to a human neuropilin-2B (NRP2b) variant 4 (v4) poly peptide and/or a human NRP2b variant 5 (v5) polypeptide.

7. The antibody, or antigen-binding fragment thereof, of any one of claims 1-6, which binds to the NRP2a v1 or v2 polypeptide, or the epitope, with an affinity of about 10 pM to about 500 pM or to about 50 nM, or about, at least about, or no more than about 10, 20, 30, 40, 50, 60, 70, 80, 90, 100, 110, 120, 130, 140, 150, 160, 170, 180, 190, 200, 300, 400, 500, 600, 700, 800, 900 pM, 1 nM, 10 nM, 25 nM, or 50 nM, or optionally with an affinity that ranges from about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500 pM, about 30 pM to about 400 pM, about 30 pM to about 300 pM, about 30 pM to about 200 pM, about 30 pM to about 100 pM, about 30 pM to about 50 pM, or about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, about 100 pM to about 1 nM, about 1 nM to about 5 nM, about 5 nM to about 10 nM, about 10 nM to 25 nM, or about 25 nM to about 50 nM.

8. The antibody, or antigen-binding fragment thereof, of any one of claims 1-7, wherein binding affinity of the antibody, or antigen-binding fragment thereof, for the NRP2a v1 or v2 polypeptide is at least about 1.5, 2, 4, 6, 8, 10, 20, 40, 60, 80, 100, 200, 400, 600, 800, or 1000 times stronger that its binding affinity for a NRP2a v3 polypeptide, a NRP2b v4 polypeptide, and/or a NRP2b v5 polypeptide.

9. The antibody, or antigen-binding fragment thereof, of any one of claims 1-8, which blocks or otherwise reduces binding between the NRP2a v1 or v2 polypeptide and a ligand thereof, optionally wherein the ligand is selected from Table L1 or Table L2.

10. The antibody, or antigen-binding fragment thereof, of any one of claims 1-9, which blocks or otherwise reduces binding between the NRP2a v1 or v2 polypeptide a chemokine (C—C motif) ligand 21 (CCL21) polypeptide, optionally in an in vitro binding assay, an in vitro or ex vivo cell-based assay, or in vivo.

11. The antibody, or antigen-binding fragment thereof, of claim 10, which blocks or otherwise reduces binding between the NRP2a v1 or v2 polypeptide and the CCL21 poly peptide by about or at least about 20-100% or more (optionally about 20, 25, 30, 35, 40, 45, 50, 55, 60, 65, 70, 75, 80, 90, or 100% or more) relative to a control or reference.

12. The antibody, or antigen-binding fragment thereof, of any one of claims 1-11, which blocks or otherwise reduces binding, including dimerization, between the NRP2a v or v2 polypeptide and a C—C chemokine receptor type 7 (CCR7) polypeptide, optionally in an in vitro binding assay, an in vitro or ex vivo cell-based assay, or in vivo.

13. The antibody, or antigen-binding fragment thereof, of claim 12, which blocks or otherwise reduces binding, including dimerization, between the NRP2a v or v2 polypeptide and the CCR7 polypeptide by about or at least about 20-100% or more (optionally about 20, 25, 30, 35, 40, 45, 50, 55, 60, 65, 70, 75, 80, 90, or 10% or more) relative to a control or reference.

14. The antibody, or antigen-binding fragment thereof, of any one of claims 1-13, which modulates (optionally antagonizes) the signaling activity between the NRP2a v1 or v2 polypeptide and a CCL21 and/or CCR7 poly peptide, optionally by about or at least about 20-100% or more (optionally about 20, 25, 30, 35, 40, 45, 50, 55, 60, 65, 70, 75, 80, 90, or 100% or more) relative to a control or reference.

15. The antibody, or antigen-binding fragment thereof, of claim 14, wherein the signaling activity comprises induction of immune cell migration, optionally dendritic cells or mature T-cells, and wherein the antibody, or antigen-binding fragment thereof, reduces the signaling activity; and/or
wherein the signaling activity comprises induction of tumor cell migration, and wherein the antibody, or antigen-binding fragment thereof, reduces the signaling activity.

16. The antibody, or antigen-binding fragment thereof, of any one of claims 1-15, which comprises an IgA (including subclasses IgA1 and IgA2), IgD, IgE, IgG (including subclasses IgG1, IgG2, IgG3, and IgG4), or IgM Fc domain, optionally a human Fc domain, or a hybrid and/or variant thereof.

17. The antibody, or antigen-binding fragment thereof, of any one of claims 1-16, which comprises an IgG Fc domain with high effector function in humans, optionally an IgG1 or IgG3 Fc domain; or which comprises an IgG Fc domain with low effector function in humans, optionally an IgG2 or IgG4 Fc domain.

18. The antibody, or antigen-binding fragment thereof, of any one of claims 1-17, which comprises an IgG1 or IgG4 Fc domain, optionally selected from Table F1.

19. The antibody, or antigen-binding fragment thereof, of any one of claims 1-18, which is a monoclonal antibody.

20. The antibody, or antigen-binding fragment thereof, of any one of claims 1-19, which is a humanized antibody.

21. The antibody, or antigen-binding fragment thereof, of any one of claims 1-20, which is an Fv fragment, a single chain Fv (scFv) poly peptide, an adnectin, an anticalin, an aptamer, an avimer, a camelid antibody, a designed ankyrin repeat protein (DARPin), a minibody, a nanobody, or a unibody.

22. A therapeutic composition, comprising a pharmaceutically-acceptable carrier and an antibody, or antigen-binding fragment thereof, of any one of claims 1-21.

23. The therapeutic composition of claim 22, wherein the composition has a purity of at least about 80%, 85%, 90%, 95%, 98%, or 99% on a protein basis with respect to the at least one antibody, or antigen-binding fragment thereof, and is substantially aggregate-free.

24. The therapeutic composition of claim 22 or 23, wherein the therapeutic composition is substantially endotoxin-free.

25. The therapeutic composition of any one of claims 22-24, wherein the therapeutic composition is a sterile, injectable solution, optionally suitable for intravenous, intramuscular, subcutaneous, or intraperitoneal administration.

26. The therapeutic composition of any one of claims 22-25, further comprising at least one additional agent selected from one or more of a cancer immunotherapy agent, a chemotherapeutic agent, a hormonal therapeutic agent, and a kinase inhibitor.

27. A method of treating a disease or condition in a subject in need thereof, comprising administering to the subject the therapeutic composition according to any one of claims 22-26.

28. The method of claim 27, wherein the disease or condition is a neuropilin 2 (NRP2)-associated disease or condition, optionally an NRP2a-associated disease or condition.

29. The method of claim 27 or 28, wherein the disease or condition is selected from a cancer, an inflammatory disease, an autoimmune disease, a lymphatic disease or associated condition, a fibrotic disease, and a disease associated with reduced smooth muscle contractility.

30. The method of claim 29, wherein the disease is a cancer, optionally wherein the cancer expresses or overexpresses NRP2, optionally wherein the cancer displays NRP2-dependent growth, NRP2-dependent adhesion, NRP2-dependent migration, and/or NRP2-dependent invasion.

31. The method of claim 30, wherein the cancer expresses or overexpresses NRP2 but does not substantially express neuropilin-1 (NRP1).

32. The method of claim 30 or 31, for reducing or preventing re-emergence of a cancer in a subject in need thereof, wherein administration of the therapeutic composition enables generation of an immune memory to the cancer.

33. The method of any one of claims 30-32, wherein the subject has lymphedema.

34. The method of any one of claims 30-33, comprising administering to the subject at least one additional agent selected from one or more of a cancer immunotherapy agent, a chemotherapeutic agent, a hormonal therapeutic agent, and a kinase inhibitor.

35. The method of claim 34, wherein the at least one anti-NRPa2 antibody or antigen-binding fragment thereof and the at least one agent are administered separately, as separate compositions.

36. The method of claim 34, wherein the at least one anti-NRP2 antibody and the at least one agent are administered together as part of the same therapeutic composition, optionally as a therapeutic composition of any one of claims 46-64.

37. The method of any one of claims 34-36, wherein the cancer immunotherapy agent is selected from one or more of an immune checkpoint modulatory agent, a cancer vaccine, an oncolytic virus, a cytokine, and a cell-based immunotherapies.

38. The method of claim 37, wherein the immune checkpoint modulatory agent is a poly peptide, optionally an antibody or antigen-binding fragment thereof or a ligand, or a small molecule.

39. The method of claim 37 or 38, wherein the immune checkpoint modulatory agent comprises
(a) an antagonist of a inhibitory immune checkpoint molecule; or
(b) an agonist of a stimulatory immune checkpoint molecule.
optionally, wherein the immune checkpoint modulatory agent specifically binds to the immune checkpoint molecule.

40. The method of claim 39, wherein the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

41. The method of claim 40, wherein:
the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736), optionally wherein the cancer is selected from one or more of colorectal cancer, melanoma, breast cancer, non-small-cell lung carcinoma, bladder cancer, and renal cell carcinoma;
the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab, optionally wherein the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer;
the PD-1 antagonist is pembrolizumab and the cancer is optionally selected from one or more of melanoma, non-small cell lung cancer, small cell lung cancer, head and neck cancer, and urothelial cancer;
the antagonist is a CTLA-4 antagonist optionally selected from one or more of an antibody or antigen-binding fragment or small molecule that specifically binds thereto, ipilimumab, tremelimumab, optionally wherein the cancer is selected from one or more of melanoma, prostate cancer, lung cancer, and bladder cancer;
the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor;
the antagonist is a TDO antagonist optionally selected from one or more of an antibody or antigen-binding fragment or small molecule that specifically binds thereto, 680C91, and LM10;
the antagonist is a TIM-3 antagonist optionally selected from one or more of an antibody or antigen-binding fragment or small molecule that specifically binds thereto;
the antagonist is a LAG-3 antagonist optionally selected from one or more of an antibody or antigen-binding fragment or small molecule that specifically binds thereto, and BMS-986016;
the antagonist is a VISTA antagonist optionally selected from one or more of an antibody or antigen-binding fragment or small molecule that specifically binds thereto;
the antagonist is a BTLA, CD160, and/or HVEM antagonist optionally selected from one or more of an antibody or antigen-binding fragment or small molecule that specifically binds thereto;
the antagonist is a TIGIT antagonist optionally selected from one or more of an antibody or antigen-binding fragment or small molecule that specifically binds thereto.

42. The method of claim 39, wherein the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

43. The method of claim 42, wherein:
the agonist is an OX40 agonist optionally selected from one or more of an antibody or antigen-binding fragment or small molecule or ligand that specifically binds thereto, OX86, Fc-OX40L, and GSK3174998;
the agonist is a CD40 agonist optionally selected from one or more of an antibody or antigen-binding fragment or small molecule or ligand that specifically binds thereto, CP-870.893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma;
the agonist is a GITR agonist optionally selected from one or more of an antibody or antigen-binding fragment or small molecule or ligand that specifically binds thereto, INCAGN01876, DTA-1, and MEDI1873;
the agonist is a CD137 agonist optionally selected from one or more of an antibody or antigen-binding fragment or small molecule or ligand that specifically binds thereto, utomilumab, and 4-1BB ligand;
the agonist is a CD27 agonist optionally selected from one or more of an antibody or antigen-binding fragment or small molecule or ligand that specifically binds thereto, varlilumab, and CDX-1127 (1F5);
the agonist is a CD28 agonist optionally selected from one or more of an antibody or antigen-binding fragment or small molecule or ligand that specifically binds thereto, and TAB08; and/or
the agonist is an HVEM agonist optionally selected from one or more of an antibody or antigen-binding fragment or small molecule or ligand that specifically binds thereto.

44. The method of claim 37, wherein the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-IR), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

45. The method of claim 37, wherein the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-240I.

46. The method of claim 37, wherein the cytokine selected from one or more of interferon (IFN)-α, IL-2, IL-12, IL-7, IL-21, and Granulocyte-macrophage colony-stimulating factor (GM-CSF).

47. The method of claim 37, wherein the cell-based immunotherapy agent comprises cancer antigen-specific T-cells, optionally ex vivo-derived T-cells.

48. The method of claim 47, wherein the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

49. The method of any one of claims 34-36, wherein the at least one chemotherapeutic agent is selected from one or more of an alkylating agent, an anti-metabolite, a cytotoxic antibiotic, a topoisomerase inhibitor (type 1 or type II), and an anti-microtubule agent.

50. The method of claim 49, wherein:
the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethyhnelamine);
the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);
the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;
the topoisomerase inhibitor is selected from one or more of camptothecin, irinotecan, topotecan, etoposide, doxorubicin, mitoxantrone, teniposide, novobiocin, merbarone, and aclarubicin;
and/or the anti-microtubule agent is selected from one or more of taxanes (optionally paclitaxel and docetaxel) and vinca alkaloids (optionally vinblastine, vincristine, vindesine, vinorelbine).

51. The method of any one of claims 34-36, wherein the at least one hormonal therapeutic agent is a hormonal agonist or a hormonal antagonist.

52. The method of claim 51, wherein the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog.

53. The method of claim 51, wherein the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

54. The method of any one of claims 34-36, wherein the kinase inhibitor is selected from one or more of adavosertib, afatinib, aflibercept, axitinib, bevacizumab, bosutinib, cabozantinib, cetuximab, cobimetinib, crizotinib, dasatinib, entrectinib, erdafitinib, erlotinib, fostamatinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemurafenib.

55. The method of any one of claims 30-54, wherein the cancer is a primary cancer.

56. The method of any one of claims 30-55, wherein the cancer is a metastatic cancer, optionally a metastatic cancer that expresses NRP2a and/or NRP2b.

57. The method of any one of claims 30-56, wherein the cancer is selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, and stomach cancer.

58. The method of claim 56 or 57, wherein the metastatic cancer is selected from one or more of:
(a) a bladder cancer which has metastasized to the bone, liver, and/or lungs;
(b) a breast cancer which has metastasized to the bone, brain, liver, and/or lungs;
(c) a colorectal cancer which has metastasized to the liver, lungs, and/or peritoneum;
(d) a kidney cancer which has metastasized to the adrenal glands, bone, brain, liver, and/or lungs;
(e) a lung cancer which has metastasized to the adrenal glands, bone, brain, liver, and/or other lung sites;
(f) a melanoma which has metastasized to the bone, brain, liver, lung, and/or
skin/muscle;
(g) a ovarian cancer which has metastasized to the liver, lung, and/or peritoneum;
(h) a pancreatic cancer which has metastasized to the liver, lung, and/or peritoneum;
(i) a prostate cancer which has metastasized to the adrenal glands, bone, liver, and/or lungs;
(j) a stomach cancer which has metastasized to the liver, lung, and/or peritoneum;
(l) a thyroid cancer which has metastasized to the bone, liver, and/or lungs; and
(m) a uterine cancer which has metastasized to the bone, liver, lung, peritoneum, and/or vagina.

59. A patient care kit, comprising:
(a) an antibody, or an antigen-binding fragment thereof, according to any one of claims 1-21; and optionally
(b) at least one additional agent selected from a cancer immunotherapy agent, a chemotherapeutic agent, a hormonal therapeutic agent, and a kinase inhibitor.

60. The patient care kit of claim 59, wherein (a) and (b) are in separate therapeutic compositions.

61. The patient care kit of claim 59, wherein (a) and (b) are in the same therapeutic composition.

62. The patient care kit of any one of claims 59-61, wherein the at least one chemotherapeutic agent is selected from one or more of an alkylating agent, an anti-metabolite, a cytotoxic antibiotic, a topoisomerase inhibitor (type 1 or type II), and an anti-microtubule agent.

63. A bioassay system, comprising an antibody, or an antigen-binding fragment thereof, according to any one of claims 1-21, and a host cell line that expresses a human NRP2 polypeptide on the cell surface.

64. The bioassay system of claim 63, wherein the NRP2 polypeptide is labeled with a detectable label.

65. The bioassay system of claim 63 or 64, wherein the antibody, or antigen-binding fragment thereof, is labeled with a detectable label.

66. The bioassay system of any one of claims 63-35, wherein the NRP2 polypeptide is functionally coupled to a readout or indicator, such as a fluorescent or luminescent indicator of biological activity of the NRP2 polypeptide.

67. The bioassay system of any one of claims 63-66, wherein the NRP2 polypeptide is selected from Table N1, optionally an NRP2a v1 and/or v2 polypeptide.

68. The bioassay system of any one of claims 63-67, comprising at least one NRP2a ligand, optionally an NRP2a ligand selected from Table L1 or Table L2, optionally wherein the host cell expresses the at least one NRP2a ligand.

69. A detection system, comprising a cell that expresses a human neuropilin 2a (NRP2a) poly peptide, at least one NRP2a ligand, and a human or humanized anti-NRP2a antibody, or an antigen-binding fragment thereof, according to any one of claims 1-21, which modulates the interaction between the NRP2a polypeptide and the at least one NRP2a ligand.

70. The detection system of claim 69, wherein the anti-NRP2a antibody, or antigen-binding fragment thereof, is labeled with a detectable label.

71. The detection system of claim 69 or 70, wherein the NRP2a polypeptide is a NRP2a variant 1 and/or variant 2 polypeptide selected from Table N1.

72. The detection system of any one of claims 69-71, wherein the at least one NRP2a ligand is selected from Table L1 or Table L2.

73. The detection system of any one of claims 69-72, wherein the NRP2a polypeptide and/or the at least one NRP2a ligand is/are functionally coupled to a readout or indicator, such as a fluorescent or luminescent indicator of biological activity of the NRP2a polypeptide or the at least one NRP2a ligand.

74. A cellular composition, comprising an engineered population of cells in which at least one cell comprises one or more polynucleotides encoding a human or humanized anti-NRP2a antibody, or antigen-binding fragment thereof, according to any one of claims 1-21, wherein the cells are capable of growing in a serum-free medium.

75. A cellular growth device, comprising a human or humanized anti-NRP2a antibody, or an antigen-binding fragment thereof, according to any one of claims 1-21, an engineered population of cells in which at least one cell comprises one or more polynucleotides encoding said anti-NRP2a antibody, or antigen-binding fragment thereof, at least about 10 liters of a serum-free growth medium, and a sterile container.


SEQUENCE LISTING (171 sequences)

SEQ ID NO: 1 - protein, 10 aa, synthetic construct
  REGION 1..10: note = aNRP2-37v2 VH CDR1
GFTFSDYALS

SEQ ID NO: 2 - protein, 17 aa, synthetic construct
  REGION 1..17: note = aNRP2-37v2 VH CDR2
YISSGGDYIYYADTVRG

SEQ ID NO: 3 - protein, 6 aa, synthetic construct
  REGION 1..6: note = aNRP2-37v2 VH CDR3
GGQDDY

SEQ ID NO: 4 - protein, 15 aa, synthetic construct
  REGION 1..15: note = aNRP2-37v2 VL CDR1
SSQSLVHSNGNTYLQ

SEQ ID NO: 5 - protein, 7 aa, synthetic construct
  REGION 1..7: note = aNRP2-37v2 VL CDR2
KVSNRFS

SEQ ID NO: 6 - protein, 9 aa, synthetic construct
  REGION 1..9: note = aNRP2-37v2 VL CDR3
SQSTHVPFT

SEQ ID NO: 7 - protein, 10 aa, synthetic construct
  REGION 1..10: note = aNRP2-400v2 VH CDR1
GYTFRSYGIS

SEQ ID NO: 8 - protein, 17 aa, synthetic construct
  REGION 1..17: note = aNRP2-400v2 VH CDR2
EIYPRSGNTYYDENFKG

SEQ ID NO: 9 - protein, 17 aa, synthetic construct
  REGION 1..17: note = aNRP2-400v2 VH CDR3
SSITAVVAIPYYYAMDY

SEQ ID NO: 10 - protein, 11 aa, synthetic construct
  REGION 1..11: note = aNRP2-400v2 VL CDR1
SASQGISNYLN

SEQ ID NO: 11 - protein, 7 aa, synthetic construct
  REGION 1..7: note = aNRP2-400v2 VL CDR2
YTSSLHS

SEQ ID NO: 12 - protein, 9 aa, synthetic construct
  REGION 1..9: note = aNRP2-400v2 VL CDR3
QQYSKLPHT

SEQ ID NO: 13 - protein, 10 aa, synthetic construct
  REGION 1..10: note = aNRP2-401v2 VH CDR1
GFTFSDYGMH

SEQ ID NO: 14 - protein, 17 aa, synthetic construct
  REGION 1..17: note = aNRP2-401v2 VH CDR2
TISRDINTVYYADTVKG

SEQ ID NO: 15 - protein, 6 aa, synthetic construct
  REGION 1..6: note = aNRP2-401v2 VH CDR3
GGYTDY

SEQ ID NO: 16 - protein, 16 aa, synthetic construct
  REGION 1..16: note = aNRP2-401v2 VL CDR1
RSSQSLVHSNGNTYLY

SEQ ID NO: 17 - protein, 7 aa, synthetic construct
  REGION 1..7: note = aNRP2-401v2 VL CDR2
KVSNRFS

SEQ ID NO: 18 - protein, 8 aa, synthetic construct
  REGION 1..8: note = aNRP2-401v2 VL CDR3
SQSTHVLT

SEQ ID NO: 19 - protein, 10 aa, synthetic construct
  REGION 1..10: note = aNRP2-402v2 VH CDR1
GFTFSNYAMS

SEQ ID NO: 20 - protein, 17 aa, synthetic construct
  REGION 1..17: note = aNRP2-402v2 VH CDR2
SISDGGSYTYYPDNVKG

SEQ ID NO: 21 - protein, 11 aa, synthetic construct
  REGION 1..11: note = aNRP2-402v2 VH CDR3
DGGPREGYFDV

SEQ ID NO: 22 - protein, 16 aa, synthetic construct
  REGION 1..16: note = aNRP2-402v2 VL CDR1
RSSQSIVHSNGNTYLE

SEQ ID NO: 23 - protein, 7 aa, synthetic construct
  REGION 1..7: note = aNRP2-402v2 VL CDR2
KVSNRFS

SEQ ID NO: 24 - protein, 9 aa, synthetic construct
  REGION 1..9: note = aNRP2-402v2 VL CDR3
FQGSHVPYT

SEQ ID NO: 25 - protein, 10 aa, synthetic construct
  REGION 1..10: note = aNRP2-403v2 VH CDR1
GYTFTSYWMH

SEQ ID NO: 26 - protein, 17 aa, synthetic construct
  REGION 1..17: note = aNRP2-403v2 VH CDR2
RIDPNSGDTKYNEKFKS

SEQ ID NO: 27 - protein, 8 aa, synthetic construct
  REGION 1..8: note = aNRP2-403v2 VH CDR3
SYDYALEY

SEQ ID NO: 28 - protein, 16 aa, synthetic construct
  REGION 1..16: note = aNRP2-403v2 VL CDR1
RSSQSLVHSNGNTYLH

SEQ ID NO: 29 - protein, 7 aa, synthetic construct
  REGION 1..7: note = aNRP2-403v2 VL CDR2
KVSNRFS

SEQ ID NO: 30 - protein, 9 aa, synthetic construct
  REGION 1..9: note = aNRP2-403v2 VL CDR3
SQNTRVPRT

SEQ ID NO: 31 - protein, 10 aa, synthetic construct
  REGION 1..10: note = aNRP2-404v2 VH CDR1
GYTFTDYNMH

SEQ ID NO: 32 - protein, 17 aa, synthetic construct
  REGION 1..17: note = aNRP2-404v2 VH CDR2
YIYPYNGDSGYNQRFKS

SEQ ID NO: 33 - protein, 5 aa, synthetic construct
  REGION 1..5: note = aNRP2-404v2 VH CDR3
LGRGY

SEQ ID NO: 34 - protein, 16 aa, synthetic construct
  REGION 1..16: note = aNRP2-404v2 VL CDR1
KSSQSLLDSDGKTYLH

SEQ ID NO: 35 - protein, 7 aa, synthetic construct
  REGION 1..7: note = aNRP2-404v2 VL CDR2
LVSKLDS

SEQ ID NO: 36 - protein, 9 aa, synthetic construct
  REGION 1..9: note = aNRP2-404v2 VL CDR3
WQGTHFPWT

SEQ ID NO: 37 - protein, 10 aa, synthetic construct
  REGION 1..10: note = aNRP2-405v2 VH CDR1
GFTFRRYAMS

SEQ ID NO: 38 - protein, 17 aa, synthetic construct
  REGION 1..17: note = aNRP2-405v2 VH CDR2
TITSGGSYTYYLDSVKG

SEQ ID NO: 39 - protein, 9 aa, synthetic construct
  REGION 1..9: note = aNRP2-405v2 VH CDR3
HGIYGGFDY

SEQ ID NO: 40 - protein, 16 aa, synthetic construct
  REGION 1..16: note = aNRP2-405v2 VL CDR1
RSSQSIVHSDGNTYLE

SEQ ID NO: 41 - protein, 7 aa, synthetic construct
  REGION 1..7: note = aNRP2-405v2 VL CDR2
KVSNRFS

SEQ ID NO: 42 - protein, 9 aa, synthetic construct
  REGION 1..9: note = aNRP2-405v2 VL CDR3
FQGSHVPFT

SEQ ID NO: 43 - protein, 10 aa, synthetic construct
  REGION 1..10: note = aNRP2-406v2 VH CDR1
GYSFTGYFMN

SEQ ID NO: 44 - protein, 17 aa, synthetic construct
  REGION 1..17: note = aNRP2-406v2 VH CDR2
RINPYNGDTFYNQKFKG

SEQ ID NO: 45 - protein, 9 aa, synthetic construct
  REGION 1..9: note = aNRP2-406v2 VH CDR3
EVAEVPFDY

SEQ ID NO: 46 - protein, 17 aa, synthetic construct
  REGION 1..17: note = aNRP2-406v2 VL CDR1
KSSQSLLYRSNQKNYLA

SEQ ID NO: 47 - protein, 7 aa, synthetic construct
  REGION 1..7: note = aNRP2-406v2 VL CDR2
WASTRES

SEQ ID NO: 48 - protein, 9 aa, synthetic construct
  REGION 1..9: note = aNRP2-406v2 VL CDR3
QQYYSYPPT

SEQ ID NO: 49 - protein, 10 aa, synthetic construct
  REGION 1..10: note = aNRP2-407v2 VH CDR1
GYSFTGYYMH

SEQ ID NO: 50 - protein, 17 aa, synthetic construct
  REGION 1..17: note = aNRP2-407v2 VH CDR2
RINPYNGATSYSQNFKD

SEQ ID NO: 51 - protein, 9 aa, synthetic construct
  REGION 1..9: note = aNRP2-407v2 VH CDR3
EETTAPFTY

SEQ ID NO: 52 - protein, 17 aa, synthetic construct
  REGION 1..17: note = aNRP2-407v2 VL CDR1
KSSQSLLYSSNQKNYLA

SEQ ID NO: 53 - protein, 7 aa, synthetic construct
  REGION 1..7: note = aNRP2-407v2 VL CDR2
WASTRES

SEQ ID NO: 54 - protein, 9 aa, synthetic construct
  REGION 1..9: note = aNRP2-407v2 VL CDR3
QHYYSYPPT

SEQ ID NO: 55 - protein, 10 aa, synthetic construct
  REGION 1..10: note = aNRP2-408v2 VH CDR1
GFTFSSYAMS

SEQ ID NO: 56 - protein, 16 aa, synthetic construct
  REGION 1..16: note = aNRP2-408v2 VH CDR2
SISRGSITYYPDSVKG

SEQ ID NO: 57 - protein, 8 aa, synthetic construct
  REGION 1..8: note = aNRP2-408v2 VH CDR3
EYYYAMDY

SEQ ID NO: 58 - protein, 11 aa, synthetic construct
  REGION 1..11: note = aNRP2-408v2 VL CDR1
RASQDIGSRLN

SEQ ID NO: 59 - protein, 7 aa, synthetic construct
  REGION 1..7: note = aNRP2-408v2 VL CDR2
ATSSLDS

SEQ ID NO: 60 - protein, 9 aa, synthetic construct
  REGION 1..9: note = aNRP2-408v2 VL CDR3
LQYASSPYT

SEQ ID NO: 61 - protein, 10 aa, synthetic construct
  REGION 1..10: note = aNRP2-409v3 VH CDR1
GFTFNTNAMN

SEQ ID NO: 62 - protein, 19 aa, synthetic construct
  REGION 1..19: note = aNRP2-409v3 VH CDR2
RIRTKSNNYATYYADSVKD

SEQ ID NO: 63 - protein, 11 aa, synthetic construct
  REGION 1..11: note = aNRP2-409v3 VH CDR3
LDSSGYVWFAY

SEQ ID NO: 64 - protein, 11 aa, synthetic construct
  REGION 1..11: note = aNRP2-409v3 VL CDR1
RASQDIGSRLN

SEQ ID NO: 65 - protein, 7 aa, synthetic construct
  REGION 1..7: note = aNRP2-409v3 VL CDR2
ATSSLDS

SEQ ID NO: 66 - protein, 9 aa, synthetic construct
  REGION 1..9: note = aNRP2-409v3 VL CDR3
LQYASSPYT

SEQ ID NO: 67 - protein, 115 aa, synthetic construct
  REGION 1..115: note = aNRP2-37v2 Heavy chain variable region
DVKLVESGEGLVKPGGSLKLSCAASGFTFSDYALSWVRQTPEKRLEWVAYISSGGDYIYYADTVRGRFTISRDNARNTLYLQMSSLQSEDTAIYYCTRGGQDDYWGQGTTLTVSS

SEQ ID NO: 68 - protein, 112 aa, synthetic construct
  REGION 1..112: note = aNRP2-37v2 Light chain variable region
DVVMTQTPLSLPVILGDQASISCRSSQSLVHSNGNTYLQWYLQKPGQSPKLLIYKVSNRFSGVPDRFSGSGSGTDFTLKISRVEAEDLGVYFCSQSTHVPFTFGSGTNLEIK

SEQ ID NO: 69 - protein, 126 aa, synthetic construct
  REGION 1..126: note = aNRP2-400v2 Heavy chain variable region
EVQLQESGAELARPGASVKLSCKASGYTFRSYGISWVKQRTGQGLEWIGEIYPRSGNTYYDENFKGRATLTADKSSSTAFMELRSLTSEDSAVYFCARSSITAVVAIPYYYAMDYWGQGTSVTVSS

SEQ ID NO: 70 - protein, 107 aa, synthetic construct
  REGION 1..107: note = aNRP2-400v2 Light chain variable region
DIQMTQTTSSLSASLGDRVTISCSASQGISNYLNWYQQKPDGSVKLLIYYTSSLHSGVPSRFSGSGSGTDYSLTISNLEPEDIATYYCQQYSKLPHTFGGGTKLELK

SEQ ID NO: 71 - protein, 115 aa, synthetic construct
  REGION 1..115: note = aNRP2-401v2 Heavy chain variable region
EVQLQESGGGLVKPGGSLKLSCAASGFTFSDYGMHWVRQAPEKGLEWVATISRDINTVYYADTVKGRFTISRDNAKNTLFLQVTSLRSEDTAMYYCTRGGYTDYWGQGTTLTVSS

SEQ ID NO: 72 - protein, 111 aa, synthetic construct
  REGION 1..111: note = aNRP2-401v2 Light chain variable region
DIVMTQTPLSLPVSLGDQASISCRSSQSLVHSNGNTYLYWYLQRPGQSPKLLIYKVSNRFSGVPDRFSGSGSGTDFTLKISRVEAEDLGVYFCSQSTHVLTFGGGTKLEIK

SEQ ID NO: 73 - protein, 120 aa, synthetic construct
  REGION 1..120: note = aNRP2-402v2 Heavy chain variable region
EVQLVESGGDLVKPGGSLKLSCAASGFTFSNYAMSWVRQTPEKRLEWVASISDGGSYTYYPDNVKGRFTISRDSAKKSLYLQMSHLKSEDTAMYYCTKDGGPREGYFDVWGTGTTVTVSS

SEQ ID NO: 74 - protein, 112 aa, synthetic construct
  REGION 1..112: note = aNRP2-402v2 Light chain variable region
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 75 - protein, 117 aa, synthetic construct
  REGION 1..117: note = aNRP2-403v2 Heavy chain variable region
QVQLQQPGAELVKPGASVKLSCKASGYTFTSYWMHWVKQRPGRGLEWIGRIDPNSGDTKYNEKFKSKATLTVDKPSSTAYMQVSSLTSEDSAVYYCARSYDYALEYWGQGTSVTVSS

SEQ ID NO: 76 - protein, 112 aa, synthetic construct
  REGION 1..112: note = aNRP2-403v2 Light chain variable region
DVVMTQTPLSLPVSLGDQASISCRSSQSLVHSNGNTYLHWFLQKPGQSPNLLIYKVSNRFSGVPDRFSGSGSGTDFTLKISRVEAEDLGVYFCSQNTRVPRTFGGGTKLEIK

SEQ ID NO: 77 - protein, 114 aa, synthetic construct
  REGION 1..114: note = aNRP2-404v2 Heavy chain variable region
EVQLQQSGPELVKPGASVKISCKASGYTFTDYNMHWVKQSHGKSLEWIGYIYPYNGDSGYNQRFKSEATLTVDISSSTAYMELRSLTSDDSAVYYCARLGRGYWGQGTTLTVSS

SEQ ID NO: 78 - protein, 112 aa, synthetic construct
  REGION 1..112: note = aNRP2-404v2 Light chain variable region
DVVMTQTPLTLSVTVGQPASISCKSSQSLLDSDGKTYLHWLFQRPGQSPRRLIYLVSKLDSGVPDRFTGSGSGTDFTLKISRVEAEDLGVYYCWQGTHFPWTFGGGTKLEIK

SEQ ID NO: 79 - protein, 118 aa, synthetic construct
  REGION 1..118: note = aNRP2-405v2 Heavy chain variable region
EVMLVESGGGLVKPGGSLKLSCAASGFTFRRYAMSWVRQTPEKGLEWVATITSGGSYTYYLDSVKGRITISRDNAKNTLYLQMSSLRSEDTAMYYCARHGIYGGFDYWGQGTTLTVSS

SEQ ID NO: 80 - protein, 112 aa, synthetic construct
  REGION 1..112: note = aNRP2-405v2 Light chain variable region
DVLMTQTPLSLPVSLGDQASISCRSSQSIVHSDGNTYLEWYLQKPGQSPNLLIYKVSNRFSGVPDRFSGSGSGTDFTLKISRVEAEDLGIYYCFQGSHVPFTFGSGTKLEIK

SEQ ID NO: 81 - protein, 118 aa, synthetic construct
  REGION 1..118: note = aNRP2-406v2 Heavy chain variable region
EVQLQQSGPELVKPGASVKISCKASGYSFTGYFMNWVKQSHGKSLEWIGRINPYNGDTFYNQKFKGKATLTVDKSSSTAHMALLSLTSEDSAVYYCGREVAEVPFDYWGQGTTLTVSS

SEQ ID NO: 82 - protein, 113 aa, synthetic construct
  REGION 1..113: note = aNRP2-406v2 Light chain variable region
DIVMSQSPSSLAVSVGEKVTMSCKSSQSLLYRSNQKNYLAWYQQKPGQSPKLLIYWASTRESGVPDRFTGSGSGTDFTLTISSVKAEDLAVYYCQQYYSYPPTFGAGTKLELK

SEQ ID NO: 83 - protein, 118 aa, synthetic construct
  REGION 1..118: note = aNRP2-407v2 Heavy chain variable region
EVQLQQSGPELVKPGASVTISCKASGYSFTGYYMHWVKQSHVKSLEWIGRINPYNGATSYSQNFKDKASLTIDKSSSTAYMELHSLTSEDSAVYYCAREETTAPFTYWGQGTLVTVSA

SEQ ID NO: 84 - protein, 113 aa, synthetic construct
  REGION 1..113: note = aNRP2-407v2 Light chain variable region
DIVMSQSPSSLAVSVGEKVTMSCKSSQSLLYSSNQKNYLAWYQQKPGQSPKLLIYWASTRESGVPDRFTGSGSGTDFTLTISSVQAEDLAVYFCQHYYSYPPTFGGGTKLEIK

SEQ ID NO: 85 - protein, 116 aa, synthetic construct
  REGION 1..116: note = aNRP2-408v2 Heavy chain variable region
EVKLVESGGGLVKPGGSLKLSCAASGFTFSSYAMSWVRQTPEKRLEWVASISRGSITYYPDSVKGRFTISRDNAGNILYLQMSSLRSEDTAMYYCAREYYYAMDYWGQGTSVTVSS

SEQ ID NO: 86 - protein, 107 aa, synthetic construct
  REGION 1..107: note = aNRP2-408v2 Light chain variable region
DIQMTQSPSSLSASLGERVSLTCRASQDIGSRLNWLQQEPDGTIKRLIYATSSLDSGVPKRFSGSRSGSDYSLTISSLESEDFVDYYCLQYASSPYTFGGGTKLEIK

SEQ ID NO: 87 - protein, 122 aa, synthetic construct
  REGION 1..122: note = aNRP2-409v3 Heavy chain variable region
EVQFVETGGGLVQPKGSLKLSCAASGFTFNTNAMNWVRQAPGKGLEWVARIRTKSNNYATYYADSVKDRFTISRDDSQNILYLQMNNLKTEDTAMYYCVTLDSSGYVWFAYWGQGTLVTVSA

SEQ ID NO: 88 - protein, 107 aa, synthetic construct; identical to SEQ ID NO: 86
  REGION 1..107: note = aNRP2-409v3 Light chain variable region

SEQ ID NO: 89 - protein, 909 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 90 - protein, 904 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 91 - protein, 887 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFADEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 92 - protein, 884 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 93 - protein, 879 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 94 - protein, 62 aa, Homo sapiens
PISAFAGENFKVDIPEIHEREGYEDEIDDEYEVDWSNSSSATSGSGAPSTDKEKSWLYTLDP

SEQ ID NO: 95 - protein, 57 aa, Homo sapiens
PISAFAVDIPEIHEREGYEDEIDDEYEVDWSNSSSATSGSGAPSTDKEKSWLYTLDP

SEQ ID NO: 96 - protein, 30 aa, Homo sapiens
PISAFAVDIPEIHEREGYEDEIDDEYEVDW

SEQ ID NO: 97 - protein, 23 aa, Homo sapiens
DIPEIHEREGYEDEIDDEYEVDW

SEQ ID NO: 98 - protein, 18 aa, Homo sapiens
PISAFAVDIPEIHEREGY

SEQ ID NO: 99 - protein, 11 aa, Homo sapiens
DIPEIHEREGY

SEQ ID NO: 100 - protein, 15 aa, Homo sapiens
EGYEDEIDDEYEVDW

SEQ ID NO: 101 - protein, 9 aa, Homo sapiens
PISAFAVDI

SEQ ID NO: 102 - protein, 20 aa, Homo sapiens
SGSGAPSTDKEKSWLYTLDP

SEQ ID NO: 103 - protein, 12 aa, Homo sapiens
DKEKSWLYTLDP

SEQ ID NO: 104 - protein, 12 aa, Homo sapiens
SGSGAPSTDKEK

SEQ ID NO: 105 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 106 - protein, 111 aa, Homo sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 107 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 108 - protein, 354 aa, Homo sapiens
QDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 109 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 330 aa, Homo sapiens [sequence omitted]

SEQ ID NO: 112 - protein, 107 aa, Homo sapiens [sequence omitted]

SEQ ID NO: 113 - protein, 57 aa, Homo sapiens; identical to SEQ ID NO: 95

SEQ ID NO: 114 - protein, 23 aa, Homo sapiens
DIPEIHEREGYEDEIDDEYEVDW

SEQ ID NO: 115 - protein, 12 aa, Homo sapiens
VDIPEIHEREGY

SEQ ID NO: 116 - protein, 12 aa, Homo sapiens
PEIHEREGYEDE

SEQ ID NO: 117 - protein, 12 aa, Homo sapiens
HEREGYEDEIDD

SEQ ID NO: 118 - protein, 12 aa, Homo sapiens
EGYEDEIDDEYE

SEQ ID NO: 119 - protein, 12 aa, Homo sapiens
EDEIDDEYEVDW

SEQ ID NO: 120 - protein, 12 aa, Homo sapiens
IDDEYEVDWSNS

SEQ ID NO: 121 - protein, 12 aa, Homo sapiens
EYEVDWSNSSSA

SEQ ID NO: 122 - protein, 23 aa, Homo sapiens
EGYEDEIDDEYEVDWSNSSSATS

SEQ ID NO: 123 - protein, 23 aa, Homo sapiens
EGYEDEIDDEYEGDWSNSSSSTS

SEQ ID NO: 124 - protein, 57 aa, Homo sapiens
PISAFAVDIPETHGGEGYEDEIDDEYEGDWSNSSSSTSGAGDPSSGKEKSWLYTLDP

SEQ ID NO: 125 - protein, 30 aa, Homo sapiens
PISAFAGGTLLPGTEPTVDTVPMQPIPAYW

SEQ ID NO: 126 - protein, 30 aa, Homo sapiens
PISAFAGGTLPPGTEPTVDTVPVQPIPAYW

SEQ ID NO: 127 - protein, 17 aa, unidentified
  REGION 1..17: note = aNRP2-401 CDR Consensus Sequence
  VARIANT 1: note = T, A, F, I, K, L, Q, R, or S
  VARIANT 2: note = I, A, F, G, L, N, Q, R, S, T, V, or Y
  VARIANT 3: note = S, A, F, G, H, I, K, L, P, Q, R, V, or Y
  VARIANT 4: note = R, A, G, K, L, N, P, Q, S, T, V, or Y
  VARIANT 5: note = D, A, G, Q, R, S, V, or Y
  VARIANT 6: note = I, A, E, G, H, K, L, N, P, R, S, T, or V
  VARIANT 7: note = N, A, D, I, L, P, Q, R, S, T, V, or Y
  VARIANT 8: note = T, A, G, H, L, N, P, R, S, or V
  VARIANT 9: note = V, F, G, I, L, N, P, Q, S, T, or Y
  VARIANT 10: note = Y, F, H, I, K, N, or R
  VARIANT 11: note = Y, A, D, F, G, H, I, K, L, N, P, Q, R, S, T, or V
XXXXXXXXXXXADTVKG

SEQ ID NO: 128-129 - skipped (2 entries)

SEQ ID NO: 130 - protein, 10 aa, synthetic construct
  REGION 1..10: note = aNRP2-401v5 VH CDR1
GFTFSDYGMH

SEQ ID NO: 131 - protein, 17 aa, synthetic construct
  REGION 1..17: note = aNRP2-401v5 VH CDR2
TISRDINTVYYADTVKG

SEQ ID NO: 132 - protein, 6 aa, synthetic construct
  REGION 1..6: note = aNRP2-401v5 VH CDR3
GGYTDY

SEQ ID NO: 133 - protein, 16 aa, synthetic construct
  REGION 1..16: note = aNRP2-401v5 VL CDR1
RSSQSLVHSNGNTYLY

SEQ ID NO: 134 - protein, 7 aa, synthetic construct
  REGION 1..7: note = aNRP2-401v5 VL CDR2
KVSNRFS

SEQ ID NO: 135 - protein, 8 aa, synthetic construct
  REGION 1..8: note = aNRP2-401v5 VL CDR3
SQSTHVLT

SEQ ID NO: 136 - protein, 10 aa, synthetic construct
  REGION 1..10: note = aNRP2-401v6 VH CDR1
GFTFSDYGMH

SEQ ID NO: 137 - protein, 17 aa, synthetic construct
  REGION 1..17: note = aNRP2-401v6 VH CDR2
TISRDINTVYYADTVKG

SEQ ID NO: 138 - protein, 6 aa, synthetic construct
  REGION 1..6: note = aNRP2-401v6 VH CDR3
GGYTDY

SEQ ID NO: 139 - protein, 16 aa, synthetic construct
  REGION 1..16: note = aNRP2-401v6 VL CDR1
RSSQSLVHSNGNTYLY

SEQ ID NO: 140 - protein, 7 aa, synthetic construct
  REGION 1..7: note = aNRP2-401v6 VL CDR2
KVSNRFS

SEQ ID NO: 141 - protein, 8 aa, synthetic construct
  REGION 1..8: note = aNRP2-401v6 VL CDR3
AQSTHPLT

SEQ ID NO: 142 - protein, 10 aa, synthetic construct
  REGION 1..10: note = aNRP2-401v7 VH CDR1
GFTFSDYGMH

SEQ ID NO: 143 - protein, 17 aa, synthetic construct
  REGION 1..17: note = aNRP2-401v7 VH CDR2
TISRSINTVYYADTVKG

SEQ ID NO: 144 - protein, 6 aa, synthetic construct
  REGION 1..6: note = aNRP2-401v7 VH CDR3
GGYTDY

SEQ ID NO: 145 - protein, 16 aa, synthetic construct
  REGION 1..16: note = aNRP2-401v7 VL CDR1
RSSQSLVHSNGNTYLY

SEQ ID NO: 146 - protein, 7 aa, synthetic construct
  REGION 1..7: note = aNRP2-401v7 VL CDR2
KVSNRFS

SEQ ID NO: 147 - protein, 8 aa, synthetic construct
  REGION 1..8: note = aNRP2-401v7 VL CDR3
AQSTHPLT

SEQ ID NO: 148 - protein, 10 aa, synthetic construct
  REGION 1..10: note = aNRP2-401v8 VH CDR1
GFTFSDYGMH

SEQ ID NO: 149 - protein, 17 aa, synthetic construct
  REGION 1..17: note = aNRP2-401v8 VH CDR2
TISRDIQTVYYADTVKG

SEQ ID NO: 150 - protein, 6 aa, synthetic construct
  REGION 1..6: note = aNRP2-401v8 VH CDR3
GGYTDY

SEQ ID NO: 151 - protein, 16 aa, synthetic construct
  REGION 1..16: note = aNRP2-401v8 VL CDR1
RSSQSLVHSNGNTYLY

SEQ ID NO: 152 - protein, 7 aa, synthetic construct
  REGION 1..7: note = aNRP2-401v8 VL CDR2
KVSNRFS

SEQ ID NO: 153 - protein, 8 aa, synthetic construct
  REGION 1..8: note = aNRP2-401v8 VL CDR3
AQSTHPLT

SEQ ID NO: 154 - protein, 10 aa, synthetic construct
  REGION 1..10: note = aNRP2-402v9 VH CDR1
GFTFSNYAMS

SEQ ID NO: 155 - protein, 17 aa, synthetic construct
  REGION 1..17: note = aNRP2-402v9 VH CDR2
SISDGGSYTYYPDNVKG

SEQ ID NO: 156 - protein, 11 aa, synthetic construct
  REGION 1..11: note = aNRP2-402v9 VH CDR3
DGGPREGYFDV

SEQ ID NO: 157 - protein, 16 aa, synthetic construct
  REGION 1..16: note = aNRP2-402v9 VL CDR1
RSSQSIVHSNGNTYLE

SEQ ID NO: 158 - protein, 7 aa, synthetic construct
  REGION 1..7: note = aNRP2-402v9 VL CDR2
KVSNRFS

SEQ ID NO: 159 - protein, 9 aa, synthetic construct
  REGION 1..9: note = aNRP2-402v9 VL CDR3
FQGSHVPYT

SEQ ID NO: 160 - protein, 115 aa, synthetic construct
  REGION 1..115: note = aNRP2-401v5 Heavy chain variable region
EVQLVESGGGLVQPGGSLRLSCAASGFTFSDYGMHWVRQAPGKGLEWVSTISRDINTVYYADTVKGRFTISRDNAKNSLYLQMNSLRAEDTAVYYCARGGYTDYWGQGTTVTVSS

SEQ ID NO: 161 - protein, 111 aa, synthetic construct
  REGION 1..111: note = aNRP2-401v5 Light chain variable region
DIVMTQTPLSLSVTPGQPASISCRSSQSLVHSNGNTYLYWYLQKPGQSPQLLIYKVSNRFSGVPDRFSGSGSGTDFTLKISRVEAEDVGVYYCSQSTHVLTFGGGTKVEIK

SEQ ID NO: 162 - protein, 115 aa, synthetic construct; identical to SEQ ID NO: 160
  REGION 1..115: note = aNRP2-401v6 Heavy chain variable region

SEQ ID NO: 163 - protein, 111 aa, synthetic construct
  REGION 1..111: note = aNRP2-401v6 Light chain variable region
DIVMTQTPLSLSVTPGQPASISCRSSQSLVHSNGNTYLYWYLQKPGQSPQLLIYKVSNRFSGVPDRFSGSGSGTDFTLKISRVEAEDVGVYYCAQSTHPLTFGGGTKVEIK

SEQ ID NO: 164 - protein, 115 aa, synthetic construct
  REGION 1..115: note = aNRP2-401v7 Heavy chain variable region
EVQLVESGGGLVQPGGSLRLSCAASGFTFSDYGMHWVRQAPGKGLEWVSTISRSINTVYYADTVKGRFTISRDNAKNSLYLQMNSLRAEDTAVYYCARGGYTDYWGQGTTVTVSS

SEQ ID NO: 165 - protein, 111 aa, synthetic construct; identical to SEQ ID NO: 163
  REGION 1..111: note = aNRP2-401v7 Light chain variable region

SEQ ID NO: 166 - protein, 115 aa, synthetic construct
  REGION 1..115: note = aNRP2-401v8 Heavy chain variable region
EVQLVESGGGLVQPGGSLRLSCAASGFTFSDYGMHWVRQAPGKGLEWVSTISRDIQTVYYADTVKGRFTISRDNAKNSLYLQMNSLRAEDTAVYYCARGGYTDYWGQGTTVTVSS

SEQ ID NO: 167 - protein, 111 aa, synthetic construct; identical to SEQ ID NO: 163
  REGION 1..111: note = aNRP2-401v8 Light chain variable region

SEQ ID NO: 168 - protein, 120 aa, synthetic construct
  REGION 1..120: note = aNRP2-402v9 Heavy chain variable region
EVQLVESGGGLVKPGGSLRLSCAASGFTFSNYAMSWVRQAPGKGLEWVSSISDGGSYTYYPDNVKGRFTISRDNAKNSLYLQMNSLRAEDTAVYYCARDGGPREGYFDVWGKGTTVTVSS

SEQ ID NO: 169 - protein, 112 aa, synthetic construct
  REGION 1..112: note = aNRP2-402v9 Light chain variable region
DVVMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHVPYTFGGGTKVEIK

SEQ ID NO: 170 - protein, 115 aa, unidentified
  REGION 1..115: note = aNRP2-401 Humanized Heavy Chain Variant Consensus Sequence
  VARIANT 50: note = T, A, F, I, K, L, Q, R, or S
  VARIANT 51: note = I, A, F, G, L, N, Q, R, S, T, V, or Y
  VARIANT 52: note = S, A, F, G, H, I, K, L, P, Q, R, V, or Y
  VARIANT 53: note = R, A, G, K, L, N, P, Q, S, T, V, or Y
  VARIANT 54: note = D, A, G, Q, R, S, V, or Y
  VARIANT 55: note = I, A, E, G, H, K, L, N, P, R, S, T, or V
  VARIANT 56: note = N, A, D, I, L, P, Q, R, S, T, V, or Y
  VARIANT 57: note = T, A, G, H, L, N, P, R, S, or V
  VARIANT 58: note = V, F, G, I, L, N, P, Q, S, T, or Y
  VARIANT 59: note = Y, F, H, I, K, N, or R
  VARIANT 60: note = Y, A, D, F, G, H, I, K, L, N, P, Q, R, S, T, or V
  VARIANT 100: note = G, A, or S
  VARIANT 101: note = Y, F, K, L, or R
  VARIANT 102: note = T, A, G, I, L, Q, or V
  VARIANT 103: note = D, A, G, K, N, Q, R, or S
  VARIANT 104: note = D, A, G, K, N, Q, R, or S
EVQLVESGGGLVQPGGSLRLSCAASGFTFSDYGMHWVRQAPGKGLEWVSXXXXXXXXXXXADTVKGRFTISRDNAKNSLYLQMNSLRAEDTAVYYCARGXXXXXWGQGTTVTVSS

SEQ ID NO: 171 - protein, 111 aa, unidentified
  REGION 1..111: note = aNRP2-401 Humanized Light Chain Variant Consensus Sequence
  VARIANT 94: note = S, A, G, I, L, P, T, or V
  VARIANT 95: note = Q, A, G, R, or S
  VARIANT 96: note = S, A, H, K, L, Q, or T
  VARIANT 97: note = T, F, G, H, I, K, L, N, Q, R, S, V, or Y
  VARIANT 98: note = H, A, D, E, F, G, I, K, L, N, Q, R, S, T, or Y
  VARIANT 99: note = V, A, E, F, G, H, I, K, L, N, P, Q, R, S, T, or Y
  VARIANT 100: note = L, A, E, H, I, N, P, Q, S, T, or V
  VARIANT 101: note = T, A, D, E, F, G, I, K, L, N, Q, R, S, or V
DIVMTQTPLSLSVTPGQPASISCRSSQSLVHSNGNTYLYWYLQKPGQSPQLLIYKVSNRFSGVPDRFSGSGSGTDFTLKISRVEAEDVGVYYCXXXXXXXXFGGGTKVEIK